SEQUENCE LISTING (13 sequences)

SEQ ID NO: 1 - DNA, 28 bp, unidentified
  source 1..28: mol_type = unassigned DNA; note = Description of Unknown: Human or mouse oligonucleotide
tggtgctggagctggtgggtggcggaga

SEQ ID NO: 2 - DNA, 28 bp, Mus sp.
  source 1..28: mol_type = genomic DNA
cggcagaaggtgggcggtggcggcggcg

SEQ ID NO: 3 - protein, 7 aa, synthetic construct
  source 1..7: note = Description of Artificial Sequence: Synthetic peptide
LRHHLTR

SEQ ID NO: 4 - protein, 7 aa, synthetic construct
  source 1..7: note = Description of Artificial Sequence: Synthetic peptide
RRFTLSK

SEQ ID NO: 5 - protein, 7 aa, synthetic construct
  source 1..7: note = Description of Artificial Sequence: Synthetic peptide
RSDVLSE

SEQ ID NO: 6 - protein, 7 aa, synthetic construct
  source 1..7: note = Description of Artificial Sequence: Synthetic peptide
KHSTRRV

SEQ ID NO: 7 - protein, 7 aa, synthetic construct
  source 1..7: note = Description of Artificial Sequence: Synthetic peptide
RLYTLHK

SEQ ID NO: 8 - protein, 7 aa, synthetic construct
  source 1..7: note = Description of Artificial Sequence: Synthetic peptide
DRSHLTR

SEQ ID NO: 9 - protein, 7 aa, synthetic construct
  source 1..7: note = Description of Artificial Sequence: Synthetic peptide
LKQHLTR

SEQ ID NO: 10 - protein, 7 aa, synthetic construct
  source 1..7: note = Description of Artificial Sequence: Synthetic peptide
RSAHLSR

SEQ ID NO: 11 - protein, 7 aa, synthetic construct
  source 1..7: note = Description of Artificial Sequence: Synthetic peptide
TSGHLSR

SEQ ID NO: 12 - protein, 7 aa, synthetic construct
  source 1..7: note = Description of Artificial Sequence: Synthetic peptide
QSGNLAR

SEQ ID NO: 13 - protein, 7 aa, synthetic construct
  source 1..7: note = Description of Artificial Sequence: Synthetic peptide
QSGDLTR

(19) United States
(12) Patent Application Publication
(10) Pub. No.: US 2025/0268981 A1
(43) Pub. Date: Aug. 28, 2025

(54) METHODS AND COMPOSITIONS FOR MODULATION OF TAU PROTEINS

(71) Applicant: Sangamo Therapeutics, Inc., Richmond, CA (US)

(72) Inventors: Brigit E. Riley, Richmond, CA (US); Bryan Zeitler, Richmond, CA (US)

(73) Assignee: Sangamo Therapeutics, Inc., Richmond, CA (US)

(21) Appl. No.: 19/189,105

(22) Filed: Apr. 24, 2025

Related U.S. Application Data
(63) Continuation of application No. 16/591,392, filed on Oct. 2, 2019, now Pat. No. 12,318,427.
(60) Provisional application No. 62/740,162, filed on Oct. 2, 2018.

(51) Int. Cl. A61K 38/17; A61K 9/00; C12N 15/86

(52) U.S. Cl. CPC A61K 38/1709; C12N 15/86; A61K 9/0019; C12N 2750/14143

(57) ABSTRACT

The present disclosure is in the field of diagnostics and therapeutics for Alzheimer's Disease.

### CROSS-REFERENCE TO RELATED APPLICATIONS

This application is a Continuation of U.S. application Ser. No. 16/591,392, filed Oct. 2, 2019, which claims the benefit of U.S. Provisional Application No. 62/740,162, filed Oct. 2, 2018, each of which is hereby incorporated by reference in its entirety.

### SEQUENCE LISTING

The instant application contains a Sequence Listing which has been submitted electronically in XML format and is hereby incorporated by reference in its entirety. Said XML copy is 14 KB in size, has the file name “91355.03117.xml” and has a production date of Apr. 24, 2025.

### TECHNICAL FIELD

The present disclosure is in the field of compositions and methods for modulating tau expression, including for the treatment and/or prevention of tauopathies such as Alzheimer's Disease.

### BACKGROUND

Abnormal levels and/or aggregation of tau protein, a microtubule-associated protein (also referred to as MAPT) that can accumulate in neurofibrillary tangles (NFT), has been implicated in a number of conditions, collectively referred to as tauopathies. These include Alzheimer's Disease (AD), Frontotermporal dementia (FTD, see Benussi et al. (2015) Front Aging Neurosci. 7(171):1-19)), Progressive Supranuclear Palsy (PSP), intractable genetic epilepsies (e.g. Dravet syndrome, see Gheyara et al. (2014) Ann Neurol 76:443-456), traumatic brain injury (TBI) and Corticobasal degeneration (CBD, see Scholz and Bras (2015) Int J. Mol Sci 16(10):24629-24655). Previous studies have shown that a reduction of tau protein expression in adult mice using antisense oligonucleotides administered directly into the cerebral spinal fluid (CSF) caused a complete or partial reduction in tau protein levels and also protected the treated mice from chemically induced seizures in terms of seizure severity (DeVos et al. (2013) J of NeuroSci 33(31):12887). In AD, for instance, there is a direct correlation between the presence of tau NFT in the brain and cognitive decline (Spires-Jones and Hyman (2014) Neuron 82:756).

It has also been suggested that tau protein may have prion like properties, as misfolded, highly phosphorylated tau protein is more easily taken up by neurons and may propagate the disease throughout the brain, and that this misfolded tau protein isolated from brains of AD patients can be readily taken up by mouse neurons (Takeda et al. (2015) Nat Comm doi:10.1038/ncomms9490; Hyman (2014) Neuron 82:1189). Entorhinal cortex-limited expression of a tangle-associated human tau protein in a transgenic mouse model led to the misfolding of mouse tau protein and aggregation of that tau protein in neurons without any detectable human tau protein expression (de Calignon et al. (2012) Neuron 73:685-697). This work suggests that the misfolded human protein is able to ‘seed’ misfolding and cause aggregation of the mouse proteins. Further, genetic reduction or loss of endogenous mouse tau is protective against neuropathological toxicity caused by overexpression of a mutant human tau transgene (Wegmann et al. (2015) EMBO J. 34(24):3028-41).

An estimated 5.3 million Americans have Alzheimer's Disease (AD) making it one of the top ten causes of death in America, and it is estimated by the year 2050, there will be 106.2 million people worldwide with the disease (van Dijk et al. (2015) Front Neurosci 9:173). The disease is more prevalent in women (two thirds of cases), and people of African or Hispanic descent are more likely to develop AD than people of Caucasian descent. The causes of AD appear to be related to genetics (especially for early onset, 5% of cases) and environmental and lifestyle factors. Typically, the disease is diagnosed in a person's mid-sixties. Although by the time a diagnosis is made, the disease has been progressing for years or even decades. The disease progresses over time, and thus far no therapeutic interventions have been identified that curtail or reverse the effects of the disease.

Repression or activation of disease-associated genes has been accomplished through the use of engineered transcription factors. Methods of designing and using engineered zinc finger transcription factors (ZFP-TF) are well documented (see for example U.S. Pat. No. 6,534,261), and more recently both transcription activator like effector transcription factors (TALE-TF) and clustered regularly interspaced short palindromic repeat Cas 9 based transcription factors (CRISPR-Cas-TF) have also been described (see review Kabadi and Gersbach (2014) Methods 69(2):188-197). Non-limiting examples of targeted genes include phospholamban (Zhang et al. (2012)Mol Ther 20(8):1508-1515), GDNF (Laganiere et al. (2010) J. Neurosci 30(49):16469) and VEGF (Liu et al. (2001) J Biol Chem 276:11323-11334). In addition, activation of genes has been achieved by use of a CRIPSR/Cas-acetyltransferase fusion (Hilton et al. (2015) Nat Biotechnol 33(5):510-517). Engineered TFs that repress gene expression (repressors) have also been shown to be effective in treating trinucleotide disorders such as Huntingtin's Disease (HD). See, e.g., U.S. Pat. No. 8,956,828 and U.S. Patent Publication No. 2015/0335708. U.S. Patent Publication No. 2018/0153921 discloses tau modulators.

However, improved compositions and methods for the diagnosis, prevention, and/or treatment of tauopathies are still needed. Thus, compositions and methods for the prevention and/or treatment of tauopathies, including AD, are described herein.

### SUMMARY

Disclosed herein are methods and compositions for diagnosing, preventing and/or treating one or more tauopathies, such as Alzheimer's Disease (AD). In particular, provided herein are methods and compositions for modifying (e.g., modulating expression of) a tau allele so as to treat at least one tauopathy such as AD, including engineered transcription factor repressors (that repress tau protein expression). Further, these methods and compositions can be used to modify a MAPT allele for the treatment and/or prevention of other tauopathies, including AD, FTD, PSP, CBD and/or seizures. Additionally, the use of two or more tau repressors provides a surprising and unexpected synergistic effect as compared to the use of single repressors. In particular, provided herein are methods and compositions for detecting, reducing and/or eliminating tau protein aggregates in vivo in a subject with a tauopathy.

Thus, described herein are genetic modulators of a microtubule associated protein tau (MAPT) gene for use in modulating tau expression in vivo. The modulator comprises at least one fusion molecule comprising a DNA-binding domain that binds to a target site of at least 12 nucleotides in the MAPT gene; and functional domain (e.g., a transcriptional regulatory domain (such as a repression domain or an activation domain) or nuclease domain). Any DNA-binding domain can be used, including but not limited to, a zinc finger protein (ZFP), a TAL-effector domain protein (TALE), a single guide RNA (of a CRISPR system), an Argonaute protein and the like. In certain embodiments, the DNA-binding domain is a zinc finger protein DNA-binding domain, for example, a ZFP-TF, namely a fusion protein comprising a ZFP that binds specifically to a tau allele and a transcriptional repression domain (e.g., KOX, KRAB, etc.). In certain embodiments, the zinc finger protein DNA-binding domains have the recognition helices in the proteins shown in Table 1, including but not limited to, ZFPs designated 57890, 65918, 57930. In any of the compositions and methods described herein, two or more genetic modulators are used (e.g., 65918 in combination with 57890). The two or more fusion proteins may bind to different target sites and comprise the same or different functional domains. The two or more tau repressors may provide a surprising and unexpected synergistic effect as compared to the use of single repressors. Alternatively, the two or more fusion proteins as described herein may bind to the same target site but include different functional domains. In some instances, three or more fusion proteins are used, in others, four or more fusion proteins are used, while in others, 5 or more fusion proteins are used. In preferred embodiments, the two or more, three or more, four or more, or five or more fusion proteins are delivered to the cell as nucleic acids (e.g., rAAV). One or more nucleic acids (e.g., AAV vectors) may be used to deliver the tau repressors described herein. In certain embodiments, the tau repressor comprises two or more tau repressors carried by a single nucleic acid vector (e.g., an AAV vector) in which the repressor-encoding sequences are separated by a 2A (e.g., T2a) sequence. In these embodiments, the sequences encoding the two or more tau repressors may be in any order (e.g., 65918 repressor—T2a—57890 repressor or 57890 repressor—T2A—65918 repressor). In preferred embodiments, the fusion proteins cause a repression of the expression of the targeted gene. In some embodiments, two fusion proteins are administered at doses where each protein is active on its own but in combination, the repression activity is additive. In preferred embodiments, two fusion proteins are administered at doses where neither is active on its own, but in combination, the repression activity is synergistic.

The genetic modulators described herein may be provided to the subject in any form, including in polynucleotide and/or protein form as well as pharmaceutical compositions comprising such polynucleotides and/or proteins.

In some aspects, the genetic modulators (or a component thereof, for example the DNA binding protein) is provided in polynucleotide form. In certain embodiments, the polynucleotide is a gene delivery vector comprising any of the polynucleotides (e.g., encoding the genetic modulators (repressors)) as described herein. In certain embodiments, the vector is an adenovirus vector (e.g., an Ad5/F35 vector), a lentiviral vector (LV) including integration competent or integration-defective lentiviral vectors, or an adenovirus associated viral vector (AAV). In certain embodiments, the genetic modulator(s) are carried on at least one AAV vector (or pseudotype or variant thereof), including but not limited to one or more AAV1, AAV2, AAV3, AAV4, AAV5, AAV6, AAV8, AAV 8.2, AAV9, AAV rh10, pseudotypes of these vectors (e.g., as AAV2/8, AAV2/5, AAV2/6, AAV2/9, etc.), including AAV vector variants known in the art (e.g. U.S. Pat. Nos. 9,585,971 and 7,198,951; U.S. Patent Publication No. 2017/0119906). In some embodiments, the AAV vector is an AAV variant capable of crossing the blood-brain barrier (e.g. U.S. Pat. No. 9,585,971).

In certain embodiments, provided herein are genetic modulators of tau that are comprised of one or more vectors, including viral and non-viral gene delivery vehicles (e.g., as mRNA, plasmids, AAV vectors, lentiviral vectors, Ad vectors), encoding the genetic modulators as described herein (or one or more components thereof on the same or different polynucleotides). In certain embodiments, the polynucleotide is an mRNA. In some aspects, the mRNA may be chemically modified (See e.g., Kormann et al. (2011) Nature Biotechnology 29(2):154-157). In other aspects, the mRNA may comprise a cap (e.g. an ARCA cap (see U.S. Pat. Nos. 7,074,596 and 8,153,773)). In further embodiments, the mRNA may comprise a mixture of unmodified and modified nucleotides (see U.S. Patent Publication No. 2012/0195936).

Pharmaceutical compositions and isolated cells comprising one or more of the genetic modulators, one or more polynucleotides, and/or one or more gene delivery vehicles are also provided. In certain embodiments, the pharmaceutical composition comprises two or more genetic modulators. For example, certain compositions include a nucleic acid comprising a sequence that encodes one of the tau modulating ZFPs, Cas or TALEs described herein operably linked to a regulatory sequence, combined with a pharmaceutically acceptable carrier or diluent, where the regulatory sequence allows for expression of the nucleic acid in a cell. In certain embodiments, the ZFPs, CRISPR/Cas or TALEs encoded are specific for a mutant or wild type MAPT allele. In some embodiments, pharmaceutical compositions comprise ZFPs, CRISPR/Cas or TALEs that modulate a mutant or wild type MAPT allele. Protein-based compositions include one of more ZFPs, CRISPR/Cas or TALEs as disclosed herein and a pharmaceutically acceptable carrier or diluent.

In other embodiments, methods and uses for repressing MAPT expression in a subject in need thereof, including by providing to the subject one or more polynucleotides, one or more gene delivery vehicles, and/or a pharmaceutical composition are described herein. In certain embodiments, the compositions described herein are used to repress MAPT expression in the subject, including for treatment and/or prevention of a tauopathy (e.g., by reducing the amount of tau in the subject). The compositions described herein reduce tau levels for sustained periods of time (4 weeks, 3 months, 6 months to year or more) in the brain (including but not limited to the frontal cortical lobe, the prefrontal cortex, parietal cortical lobe, occipital cortical lobe, temporal cortical lobe including by not limited to the entorhinal cortex, hippocampus, brain stem, striatum, thalamus, midbrain, cerebellum) and spinal cord (including but not limited to the lumbar, thoracic and cervical regions). The compositions described herein may be provided to the subject by any administration means, including but not limited to, intracerebroventricular, intrathecal, intracranial, intravenous, orbital (retro-orbital (RO)), intranasal and/or intracisternal administration. Kits comprising one or more of the compositions (e.g., genetic modulators, polynucleotides, pharmaceutical compositions and/or cells) as described herein as well as instructions for use of these compositions are also provided.

Thus, provided herein are methods for treating and/or preventing a tauopathy such as Alzheimer's Disease or seizure using the methods and compositions described herein. In some embodiments, the methods involve compositions where the polynucleotides and/or proteins (or pharmaceutical compositions comprising the polynucleotides and/or proteins) may be delivered using a viral vector, a non-viral vector (e.g., plasmid) and/or combinations thereof. Administration of compositions as described herein (proteins, polynucleotides, cells and/or pharmaceutical compositions comprising these proteins, polynucleotides and/or cells) result in a therapeutic (clinical) effect, including, but not limited to, amelioration or elimination of any the clinical symptoms associated with AD, tauopathies or seizure as well as an increase in function and/or number of CNS cells (e.g., neurons, astrocytes, myelin, etc.). In certain embodiments, the compositions and methods described herein reduce tau gene and/or protein expression (as compared to controls not receiving the artificial repressors as described herein) by at least 30%, or 40%, preferably by at least 50%, even more preferably by at least 70%, or at least 80% or at least 90%, or at least 95% or greater that 95%. In some embodiments, at least 50% reduction is achieved.

Delivery may be to any brain region, for example, the hippocampus or entorhinal cortex by any suitable means including via the use of a cannula or any other delivery technology. Any AAV vector may provide widespread delivery of the repressor to the brain of the subject, including via anterograde and retrograde axonal transport to brain regions not directly administered by the vector (e.g., delivery to the putamen results in delivery to other structures such as the cortex, substantia nigra, thalamus, etc.). In certain embodiments, the subject is a human and in other embodiments, the subject is a non-human primate (NHP). The administration may be in a single dose, or in a series of doses given at the same time, or in multiple administrations (at any timing between administrations).

Furthermore, in any of the methods described herein, the repressors can be delivered at any concentration (dose) that provides the desired effect. In preferred embodiments, the repressor is delivered using an adeno-associated virus (AAV) vector at 10,000-500,000 vector genomes/cell (or any value therebetween). In certain embodiments, the repressor is delivered using a lentiviral vector at a multiplicity of infection (MOI) between 250 and 1,000 (or any value therebetween). In other embodiments, the repressor is delivered using a plasmid vector at 0.01-1,000 ng/100,000 cells (or any value therebetween). In other embodiments, the repressor is delivered as mRNA at 0.01-3000 ng/number of cells (e.g., 50,000-200,000 (e.g., 100,000) cells (or any value therebetween). In other embodiments, the repressor is delivered using an adeno-associated virus (AAV) vector at a fixed volume of 1-300 μl to the brain parenchyma at 1E10-1E14 VG/ml (or any value therebetween). In other embodiments, the repressor is delivered using an adeno-associated virus (AAV) vector at a fixed volume of 0.5-10 ml to the CSF at 1E10-1E14 VG/ml (or any value therebetween).

Thus, in other aspects, described herein is a method of preventing and/or treating a tauopathy (e.g., AD) in a subject, the method comprising administering a repressor of a tau allele to the subject using one or more AAV vectors. In certain embodiments, the AAV encoding the repressor is administered to the CNS (brain and/or CSF) via any delivery method including but not limited to, intracerebroventricular, intrathecal, intracranial, intravenous, intranasal, retro-orbital, or intracisternal delivery. In other embodiments, the AAV encoding the repressor is administered directly into the parenchyma (e.g., hippocampus and/or entorhinal cortex) of the subject. In other embodiments, the AAV encoding the repressor is administered intravenously (IV). In any of the methods described herein, the administering may be done once (single administration) or may be done multiple times (with any time between administrations) at the same or different doses per administration. When administered multiple times, the same or different dosages and/or delivery vehicles of modes of administration may be used (e.g., different AAV vectors administered IV and/or ICV). The methods include methods of reducing the aggregation of tau in the subject (e.g., reducing NFTs characteristic of tau aggregation) for example in AD neurons of a subject with AD; methods of reducing apoptosis in a neuron or population of neurons (e.g., an AD neuron or population of AD neurons); methods of reducing neuronal hyperexcitability; methods of reducing amyloid beta induced toxicity (e.g. synapse loss and/or neuritic dystrophy); and/or methods of reducing loss to one or more cognitive functions in AD subjects, all in comparison with a subject not receiving the method, or in comparison to the subject themselves prior to receiving the methods. Thus, the methods described herein result in reduction in biomarkers and/or symptoms of tauopathies, including one or more of the following: neurotoxicity, pathological tau species (e.g., NFTs or phosphorylated tau), neurofilament light chain (Nfl), CSF tau, gliosis, dystrophic neurites, spine loss, excitotoxicity, cortical and hippocampal shrinkage, volumetric changes associated with the region impacted by the specific tauopathy, dendritic tau accumulation, cognitive (e.g., the radial arm maze and the Morris water maze in rodent models, fear conditioning, etc.), and/or motor deficits.

In some aspects, methods and compositions for reducing the amount of a pathogenic tau species in a cell are provided. In some embodiments, the methods result in a reduction of hyperphosphorylated tau. In some instances, the reduction of hyperphosphorylated tau results in a reduction of soluble or granular tau. In other embodiments, the reduction of pathogenic tau species decreases tau aggregation and causes a reduction in neurofibrillary tangles (NFTs) as compared to a cell or subject that has not been treated following the methods and/or with the compositions of described herein. In further embodiments, the methods of reversing the amount of NFTs observed in a cell are provided. In still further embodiments, the methods and compositions of the invention cause a slowing of the propagation of pathogenic tau species (e.g., NFTs, hyperphosphorylated tau) within the brain of a subject. In some embodiments, propagation of pathogenic tau across the brain is halted, and in other embodiments, propagation of pathogenic tau across the brain is reversed. In further embodiments, the number of dystrophic neurites associated with amyloid β plaques in the brain is reduced. In some embodiments, the number of dystrophic neurites is reduced to the levels found in an age-matched wild type brain. In further embodiments, provided herein are methods and compositions for reducing hyperphosphorylated tau associated with amyloid plaques in the brain of a subject.

In some embodiments, following administration to the subject, the sequence encoding a genetic modulator (genetic repressor) as described herein (e.g., ZFP-TF, TALE-TF or CRISPR/Cas-TF) is inserted (integrated) into the genome while in other embodiments the sequence encoding the repressor is maintained episomally. In some instances, the nucleic acid encoding the TF fusion is inserted (e.g., via nuclease-mediated integration) at a safe harbor site comprising a promoter such that the endogenous promoter drives expression. In other embodiments, the repressor (TF) donor sequence is inserted (via nuclease-mediated integration) into a safe harbor site and the donor sequence comprises a promoter that drives expression of the repressor. In some embodiments, the sequence encoding the genetic modulator is maintained extrachromosomally (episomally) after delivery, and may include a heterologous promoter. The promoter may be a constitutive or inducible promoter. In some embodiments, the promoter sequence is broadly expressed while in other embodiments, the promoter is tissue or cell/type specific. In preferred embodiments, the promoter sequence is specific for neuronal cells. In other preferred embodiments, the promoter chosen is characterized in that it has low expression. Non-limiting examples of preferred promoters include the neural specific promoters NSE, Synapsin, CAMKiia and MECPs. Non-limiting examples of ubiquitous promoters include CMV, CAG and Ubc. Further embodiments include the use of self-regulating promoters as described in U.S. Patent Publication No. 2015/0267205.

In any of the methods described herein, the method can yield about 50% or greater, about 55% or greater, about 60% or greater, about 65% or greater, about 70% or greater, about 75% or greater, about 85% or greater, about 90% or greater, about 92% or greater, about 95% or greater, 98% or greater, or 99% or greater repression of the tau alleles in one or more AD neurons of the subject.

In certain aspects, described are methods of preventing and/or treating a tauopathy using the methods and compositions described herein. In certain embodiments, use of an artificial transcription factor (e.g., a zinc finger protein (ZFP-TF), a TALE (TALE-TF), and/or CRISPR/Cas-TF), optionally a ZFP-TF comprising a ZFP designated 65918, 57890, and/or 57930) that modulates (e.g., represses) MAPT gene expression and/or tau protein levels in the central nervous system (CNS) of a subject is used for the prevention and/or treatment of tauopathy (e.g., Alzheimer's Disease (AD), Frontotermporal Dementia, Progressive Supranuclear Palsy, traumatic brain injury (TBI), seizure disorders and/or Corticobasal Degeneration), preferably wherein the symptoms of the tauopathy are reduced or eliminated, optionally by reducing the occurrence of neural tangles in the brain of the subject. In any of the methods and uses described herein, the artificial transcription factor(s) is(are) are delivered to the CNS (e.g., brain or spinal cord) of the subject by a viral vector, such as an AAV vector (e.g., AAV9), for example intravenously or to the CNS, optionally into the striatum or hippocampus in one or both hemispheres of the brain of the subject. In certain embodiments, the AAV vector comprises a CMV or synapsin (SYN) promoter. In still further embodiments, artificial transcription factors reduce MAPT gene expression and/or tau levels in the brain of the primate subject by 50% or more, optionally 70% or more, and up to 99% as compared to untreated subjects. In any of the embodiments described herein, the artificial transcription factor is carried by an AAV vector comprising a CMV or SYN promoter at 6E11 rAAV vector genomes per hemisphere.

Also provided is a kit comprising one or more of the AAV tau-modulators (e.g., repressors) and/or polynucleotides comprising components of and/or encoding the tau-modulators (or components thereof) as described herein. The kits may further comprise cells (e.g., neurons), reagents (e.g., for detecting and/or quantifying tau protein, for example in CSF) and/or instructions for use, including the methods as described herein.

Thus, provided herein is a composition comprising two or more artificial zinc finger protein transcription factors (ZFP-TFs) that repress MAPT expression (e.g., the ZFPs designated 65918 in combination with 57890 or 57930). The compositions comprising two or more ZFP-TFs may repress MAPT expression 1-, 2-, 3-, 4-, 5-, 6-, 7-, 8-, 9-, 10-fold or more as compared to single repressors and/or untreated controls (subjects). The composition typically comprises one or more polynucleotides encoding the two or more ZFP-TFs are encoded by polynucleotides, which may be carried by one or more viral (e.g., AAV such as AAV9) vectors, for example a single AAV vector comprising a polynucleotide encoding both of the ZFP-TFs or separate AAV vectors encoding each of the ZFP-TFs. Any promoters may be used to drive expression of the ZFP-TFs including but not limited to CMV and/or synapsin (SYN) promoter. One or more compositions (e.g., one or more AAV vectors comprising sequences encoding the two or more ZFP-TFs) described herein may be used to for the prevention and/or treatment of tauopathy (e.g., Alzheimer's Disease (AD), Frontotermporal Dementia, Progressive Supranuclear Palsy, traumatic brain injury (TBI), seizure disorders and/or Corticobasal Degeneration) in a subject in need thereof, optionally wherein the symptoms of the tauopathy are reduced or eliminated, optionally by reducing the occurrence of neural tangles in the brain of the subject. The compositions may be administered to the subject intravenously or to the CNS (e.g., into the striatum or hippocampus in one or both hemispheres of the brain of the subject), optionally wherein the one or more AAV vectors are administered at between about 1E10 to 6E11 rAAV vector genomes per hemisphere. Expression of two or more ZFP-TFs of the composition reduce MAPT gene expression and/or tau levels in the brain of the primate subject by 50% or more, optionally 70% or more, and up to 99% as compared to untreated subjects.

### BRIEF DESCRIPTION OF THE DRAWINGS

FIG. 1 is a graph depicting tau expression and ZFP transcript levels in an exemplary sample (“punch 088”) taken from the caudal hippocampus of non-human primates (NHPs) treated with tau repressors as described herein. The top plot shows % normalized tau repression and the bottom plot shows ZFP mRNA copies (transcripts/ng RNA). The left half of the graph represents data from Punch 088 obtained from the left hemisphere of each animal in the study, and the right half shows data from Punch 088 obtained from the right hemisphere of each animal in the study. The promoters used in the different constructs are shown along the bottom where “CMV” indicates the use of the CMV promoter and “SYN” indicates use of the synapsin promoter.

FIG. 2 are graphs depicting tau expression and ZFP levels in exemplary samples (“punch 088” in the top panel taken from the caudal hippocampus and “punch 035” in the bottom panel taken from the rostral hippocampus) taken from NHPs treated with tau repressors as described herein. The top plot in each panel shows % normalized tau repression and the bottom plot in each panel shows ZFP mRNA levels (copies/ng mRNA).

FIG. 3A through FIG. 3C show graphs depicting ZFP levels and unscaled tau protein expression levels (top graphs), scaled to the average of the three vehicle-treated animals (middle graphs), or scaled to the average of the vehicle-treated animals and ZFP-treated animals without detectable ZFP expression (bottom graphs) of the indicated NHP brain samples. FIG. 3A shows results for punch 037; FIG. 3B shows results for punch 039; and FIG. 3C shows results for punch 061, each sample (punch) from the hippocampus. The top plot in each panel shows % normalized tau repression (left axis) and the bottom plot in each panel shows ZFP mRNA levels (copies/ng mRNA) (right axis).

FIG. 4 is a graph depicting tau expression and ZFP levels from 74 punches taken from various brain slices ordered rostral to caudal (5, 6, 7, 8 and 9 in each hemisphere) in an exemplary NHP subject (NHP07) treated with tau repressors as described herein. The top plot in each panel shows % normalized tau repression (left axis) and the bottom plot in each panel shows ZFP mRNA levels (copies/ng mRNA) (right axis).

FIG. 5A through FIG. 5C depict tau expression and ZFP levels and corresponding MRI images in control and treated NHP subjects. FIG. 5A and FIG. 5B are graphs showing tau expression and ZFP levels, with a focus on punches from brain slice 7 showing the cortex and hippocampus in control (“Vehicle” as shown in the FIG. 5A) and a NHP subject treated with tau repressors 65918 (“918”) and 57890 (“890”) carried by an AAV vector (AAV9) where expression of the repressors (918 and 890) is driven by the synapsin (SYN1) promoter and the repressors are linked by a T2A cleavage peptide. The top plot in FIG. 5A and FIG. 5B show % normalized tau repression and the bottom plot in FIG. 5A and FIG. 5B show ZFP mRNA levels (copies/ng mRNA). FIG. 5C depicts MRI scans from the same control (left image) and treated (right image) subjects at the level of the injection track. A co-infused gadolinium tracer is evident in the hippocampal region in both hemispheres.

FIG. 6A through FIG. 6O are graphs depicting tau expression and ZFP levels in samples taken from control and treated NHP subjects. FIG. 6A through FIG. 6C show results from control subjects (NHP01 as shown in FIG. 6A, NHP02 as shown in FIG. 6B and NHP03 as shown in FIG. 6C). FIG. 6D through FIG. 6F show results from NHP subjects (NHP04 as shown in FIG. 6D, NUP05 as shown in FIG. 6E and NHP06 as shown in FIG. 6F) treated with genetic repressors 65918 (“918”) and 57890 (“890”) carried by an AAV vector (AAV9) where expression of the repressor (918 and 890) is driven by the synapsin (SYN1) promoter (“SYN1.918-890”). FIG. 6G through FIG. 6J show results from subjects (NHP07 and NHP08) treated with genetic repressors 65918 (“918”) and 57890 (“890”) carried by an AAV vector (AAV9) where expression of the repressor (918 and 890) is driven by a CMV promoter (“CMV.918-890”) (FIG. 6G and FIG. 6H), and subjects (NHP09 and NHP10) treated with the genetic repressor 57930 (“930”) carried by an AAV vector (AAV9) where expression of the repressor (930) is driven by a synapsin (SYN1) promoter (“SYN1.930”) (FIG. 6I and FIG. 6J). FIG. 6K through FIG. 6M show results from subjects (NHP 11 as shown in FIG. 6K, NHP12 as shown in FIG. 6L and NHP13 as shown in FIG. 6M) treated with 57890 (“890”) carried by an AAV vector (AAV9) where expression of the repressor is driven by the synapsin (SYN1) promoter (“SYN1.890”). FIG. 6N and FIG. 6O show results from subjects (NHP14 as shown in FIG. 6N and NHP15 as shown in FIG. 6O) treated with 65918 (“918”) carried by an AAV vector (AAV9) where expression of the repressor is driven by the synapsin (SYN1) promoter (“SYN1.918”). The top plot in each graph shows % normalized tau repression and the bottom plot in each graph shows ZFP mRNA levels (copies/ng mRNA).

FIG. 7A through FIG. 7F are graphs depicting a composite analysis of ZFP levels and unscaled tau expression levels (FIG. 7A and FIG. 7D), or tau expression scaled to the average of the three vehicle-treated animals (FIG. 7B and FIG. 7E), or tau expression scaled to the average of the vehicle-treated animals and ZFP-treated animals without detectable ZFP expression (FIG. 7C and FIG. 7F) of the indicated NHP brain samples (punch ID on X-axis). FIG. 7A, FIG. 7B and FIG. 7C show control subjects (“vehicle”) and FIG. 7D, FIG. 7E and FIG. 7F show all treated subjects (“All AAV-treated NHPs”).

FIG. 8A through FIG. 8D are graphs depicting a composite analysis of tau expression (FIG. 8A and FIG. 8C) and ZFP levels (FIG. 8B and FIG. 8D) in control (FIG. 8A and FIG. 8B) and treated subjects (FIG. 8C and FIG. 8D) using the method of scaling tau expression to the average of the vehicle-treated animals and ZFP-treated animals without detectable ZFP expression.

FIG. 9A and FIG. 9B are graphs depicting tau expression in the left (FIG. 9A) and right (FIG. 9B) hemispheres of control and treated subject as indicated. “VEH” indicates control subject who were administered vehicle only; “SYN918-890” refers to subjects receiving AAV vectors encoding the 65918 and 57890 genetic repressors, where expression is driven by a synapsin promoter; “CMV918-890” refers to subjects receiving AAV vectors encoding the 65918 and 57890 genetic repressors, where expression is driven by a CMV promoter; “SYN930” refers to subjects receiving AAV vectors encoding the 57930 genetic repressor, where expression is driven by a synapsin promoter; “SYN890” refers to subjects receiving AAV vectors encoding the 57890 genetic repressor, where expression is driven by a synapsin promoter; and “SYN918” refers to subjects receiving AAV vectors encoding the 65918 genetic repressor, where expression is driven by a synapsin promoter. For this analysis, normalized tau expression was scaled to the average of the tau levels measured from vehicle-treated animals and ZFP-treated animals without detectable ZFP expression for each punch.

FIG. 10A through FIG. 10F are graphs depicting tau expression levels where ZFP transcript levels in the indicated NHP subjects were less than 1E4 ZFP transcripts (copies/ng mRNA) (FIG. 10A shows levels in the left hemisphere and FIG. 10B shows levels in the right hemisphere); between 1E4 and 1E5 transcripts (copies/ng mRNA) (FIG. 10C shows levels in the left hemisphere and FIG. 10D shows levels in the right hemisphere); and greater than 1E5 transcripts (copies/ng mRNA) (FIG. 10E shows levels in the left hemisphere and FIG. 10F shows in the right hemisphere). For this analysis, normalized tau expression for a given punch was scaled to the average of the tau levels measured from vehicle-treated animals and ZFP-treated animals without detectable ZFP expression.

FIG. 11A through FIG. 11D are graphs depicting tau expression levels in the left and right hemispheres where ZFP transcript levels in the indicated NHP subjects are show for ZFP transcript levels (FIG. 11A) were less than 1E4 ZFP transcripts (copies/ng mRNA) (FIG. 11B); between 1E4 and 1E5 transcripts (copies/ng mRNA) (FIG. 11C); and greater than 1E5 transcripts (copies/ng mRNA) (FIG. 11D). For this analysis, normalized tau expression for a given punch was scaled to the average of the tau levels measured from vehicle-treated animals and ZFP-treated animals without detectable ZFP expression.

FIG. 12 are graphs depicting correlation plots showing tau expression and ZFP transcript levels in control subjects (left panel—“Vehicle”) and subjects treated with AAV vectors encoding the 65918 and 57890 genetic repressors, where expression is driven by a synapsin promoter (“hSYN1.5789-65918”). The limit of the ZFP qRT-PCR assay for absolute quantitation is approximately 1E2 transcripts/ng mRNA which is indicated by Below Limit of Quantitation (BLOQ). For this analysis, tau expression is scaled to the average of the Vehicle-treated animals and ZFP-treated animals without detectable ZFP expression.

FIG. 13A through FIG. 13E are correlation plots showing tau expression at the indicated ZFP transcript levels for the indicated treatment groups. FIG. 13A shows results for groups treated with hSYN1.57890-65918; FIG. 13B shows results for groups treated with CMV.57890-65918; FIG. 13C shows results for groups treated with hSYN1.57930; FIG. 13D shows results for groups treated with hSYN1.57890; and FIG. 13E shows results for groups treated with hSYN.65918. For this analysis, tau expression levels for each punch are scaled to the average of the three vehicle-treated animals. The top panels of each of FIG. 13A through 13E show the relationship across all ZFP mRNA levels; the bottom panels of each of FIG. 13A through 13E show only the range of ZFP expression associated with tau reduction, approximately 1E4-1E6 ZFP transcripts/ng mRNA. Also shown are R-squared and P values. For this analysis, tau expression is scaled to the average of the vehicle-treated animals and ZFP-treated animals without detectable ZFP expression.

FIG. 14 are correlation plots showing tau expression and ZFP transcript levels in control subjects (left panel—“Vehicle”) and subjects treated with AAV vectors encoding the 65918 and 57890 genetic repressors, where expression is driven by a synapsin promoter (“hSYN1.5789-65918”). The limit of the ZFP qRT-PCR assay for absolute quantitation is approximately 1E2 transcripts/ng mRNA which is indicated by Below Limit of Quantitation (BLOQ). In contrast to FIG. 12, for this analysis, tau expression levels are scaled to the average of the three vehicle-treated animals.

FIG. 15A through FIG. 15E are correlation plots showing tau expression at the indicated ZFP transcript levels for the indicated treatment groups: FIG. 15A shows results for groups treated with hSYN1.57890-65918; FIG. 13B shows results for groups treated with CMV.57890-65918; FIG. 15C shows results for groups treated with hSYN1.57930; FIG. 15D shows results for groups treated with hSYN1.57890; and FIG. 15E shows results for groups treated with hSYN.65918. For this analysis, tau expression levels for each punch are scaled to the average of the three vehicle-treated animals. The top panels of each of FIG. 15A through FIG. 15E show the relationship across all ZFP mRNA levels; the bottom panels of each of FIG. 15A through FIG. 15E show only the range of ZFP expression associated with tau reduction, approximately 1E4-1E6 ZFP transcripts/ng mRNA. Also shown are R-squared and P values. In contrast to FIG. 13A through FIG. 13E, for this analysis, tau expression levels are scaled to the average of the three vehicle-treated animals.

FIG. 16 are correlation plots showing tau expression and ZFP transcript levels in control subjects (left panel—“Vehicle”) and subjects treated with AAV vectors encoding the 65918 and 57890 genetic repressors, where expression is driven by a synapsin promoter (“hSYN1.5789-65918”). The limit of the ZFP qRT-PCR assay for absolute quantitation is approximately 1E2 transcripts/ng mRNA which is indicated by Below Limit of Quantitation (BLOQ). For this analysis, tau expression levels were not scaled to correct for baseline tau levels in untreated or ZFP-negative animals.

FIG. 17A through FIG. 17E are correlation plots showing tau expression at the indicated ZFP transcript levels for the indicated treatment groups: FIG. 17A shows results for groups treated with hSYN1.57890-65918; FIG. 17B shows results for groups treated with CMV.57890-65918; FIG. 17C shows results for groups treated with hSYN1.57930; FIG. 17D shows results for groups treated with hSYN1.57890; and FIG. 17E shows results for groups treated with hSYN.65918. For this analysis, tau expression levels for each punch are scaled to the average of the three Vehicle-treated animals. The top panels of each of FIG. 17A through FIG. 17E show the relationship across all ZFP mRNA levels; the bottom panels of each of FIG. 17A through FIG. 17E show only the range of ZFP expression associated with tau reduction, approximately 1E4-1E6 ZFP transcripts/ng mRNA. Also shown are R-squared and P values. For this analysis, tau expression levels were not scaled to correct for baseline tau levels in untreated or ZFP-negative animals.

FIG. 18A through FIG. 18D are bar graphs depicting results from humanized Tau mice treated with tau ZFP-TFs as described herein. FIG. 18A shows relative (left graph) and absolute (right graph) ZFP levels of mice treated with the indicated constructs. As shown, mice treated with the synergistic combination of 65918 and 57890 showed expression levels 2× higher than 57890 alone. FIG. 18B shows human tau (“hTAU”, left graph), mouse tau (“mTAU”, middle graph) and GFP (right graph) expression levels in mice treated with the indicated constructs. As shown, human tau mRNA was repressed by ˜90% by 65918-T2a-57890 as compared to ˜60% repression by 57890. Likewise, mouse tau mRNA was repressed ˜87% by 65918-T2a-57890 as compared to 81% repression by 57890 alone. FIG. 18C depicts expression of hTAU (left graph) and human Saitohin (“hSTH”, right graph) in the humanized tau mice treated under the indicated conditions. Similar levels of human tau and STH reduction were observed consistent with the fact that STH is nested within tau and, therefore, the two genes are coregulated. FIG. 18D shows expression levels of GFAP (astrocyte marker) (left graph), IBA1 (microglia marker) (middle graph) and NeuN (neuronal marker) (right graph) of humanized Tau mice treated with the indicated constructs.

FIG. 19A through FIG. 19K are graphs depict increased microglial and astrocyte marker expression levels in non-human primates (NHP) following treatment with the indicated tau-specific ZFP-TF expressed from a ZFP-TF construct including either a CMV or synapsin (SYN) promoter. FIG. 19A through FIG. 19F show ionized calcium-binding adapter molecule 1 (IBA1) levels, a marker of microglia in the indicated treatment groups: FIG. 19A shows group treated with hSYN1.57890-65918; FIG. 19B shows results for groups treated with CMV.57890-65918; FIG. 19C shows results for groups treated with hSYN1.57930; FIG. 19D shows results for groups treated with hSYN1.57890; FIG. 19E shows results for groups treated with hSYN.65918; and FIG. 19F shows side by side results for groups treated with CMV.57890-65918 (left panel) and hSYN1.57890-65918 (right panel). Microglial activation is indicative of an inflammatory response. FIG. 19G through FIG. 19K show astrocyte activation (as measured by GFAP levels) in the indicated treatment groups: FIG. 19G shows results for groups treated with hSYN1.57890-65918; FIG. 19H shows results for groups treated with CMV.57890-65918; FIG. 19I shows results for groups treated with hSYN1.57930; FIG. 19J shows results for groups treated with hSYN1.57890; and FIG. 19K shows results for groups treated with hSYN.65918. As shown, no ZFP dependent elevation in microglial or astrocyte levels was seen for any of the constructs comprising the SYN promoter, while microglial and astrocyte markers were elevated with constructs comprising the CMV promoter.

FIG. 20A through FIG. 20D are graphs showing bulk levels of IBA1 (FIG. 20A and FIG. 20B) and GFAP (FIG. 20C and FIG. 20D) in left (“L”) (FIG. 20A and FIG. 20C) and right (“R”) (FIG. 20B and FIG. 20D) hemispheres of the primates treated as indicated. As shown while IBA1 and GFAP levels vary between NHP, no major bulk effect was observed for any treatment group.

FIG. 21A through FIG. 21E are graphs depicting normalized expression of the housekeeping gene EIF4a2 to ZFP in subjects treated under the indicated conditions. FIG. 21A shows results for groups treated with hSYN1.57890-65918; FIG. 21B shows results for groups treated with CMV.57890-65918; FIG. 21C shows results for groups treated with hSYN1.57930; FIG. 21D shows results for groups treated with hSYN1.57890; and FIG. 21E shows results for groups treated with hSYN.65918. As shown, there was no correlation with the levels of a housekeeping gene (EIF4A2) and ZFP expression in any of the treatment groups.

FIG. 22 is a graph depicting total tau protein levels in human iPSC-derived (IPS) neurons under the indicated conditions. “Mock” refers to cells that were transfected with empty AAV vectors; “control” refers to cells transfected with AAV vector encoding a ZFP that does not target tau; “57930” refers to cells treated with an AAV vector encoding the 57930 repressor; “57890” refers to cells treated with an AAV vector encoding the 57890 repressor; “65918” refers to cells treated with an AAV vector encoding the 65918 repressor; and “65918/57890” refers to cells treated with an AAV vector encoding both the 65918 and 57890 repressors. All AAV constructs encoding the ZFP repressors comprised a synapsin promoter (SYN1) driving expression of the repressor. Tau protein levels evaluated by ELISA 32 days post AAV vector administration.

### DETAILED DESCRIPTION

Disclosed herein are compositions and methods for the prevention and/or treatment of tauopathies. In particular, the compositions and methods described herein are used to repress the expression of a MAPT (tau) protein to prevent or treat tauopathies such as Alzheimer's Disease (AD), Frontotermporal Dementia, Progressive Supranuclear Palsy, traumatic brain injury (TBI), seizure disorders and/or Corticobasal Degeneration. The MAPT repressors (e.g., MAPT-modulating transcription factors, such as MAPT-modulating transcription factors comprising zinc finger proteins (ZFP TFs), TALEs (TALE-TF), and/or CRISPR/Cas-TFs), modify the CNS such that the effects and/or symptoms of the tauopathy is reduced or eliminated, for example by reducing the aggregation of tau in the brain of a subject with a tauopathy (e.g., AD) and reducing the occurrence of neural tangles. In preferred embodiments, the MAPT-modulating transcription factors are delivered to the brain by a viral vector such as an AAV. AAV has been shown to be well suited for brain delivery, so use of these viral vectors to deliver MAPT modulating transcription factors is especially useful for the treatment of diseases such as Alzheimer's Disease associated with the inappropriate expression and thereby aggregation of tau protein.

The Microtubule Associated Protein Tau (MAPT) is closely linked to the pathogenesis of several neurodegenerative disorders, including Alzheimer's Disease, Progressive Supranuclear Palsy, and Frontotemporal Dementia. While genetic and antisense-based tau lowering approaches have proven efficacious and well tolerated in mice, the development of a single-administration, intracellular tau-targeted therapy remains a long-standing goal. Advances in Zinc Finger Protein (ZFP) design and AAV delivery have created new potential for a one-time, DNA-targeted therapy for neurodegenerative disease. ZFP repressors targeting the mouse, nonhuman primate (NHP), and human tau transcriptional regulatory elements were delivered to the mouse and NHP brain using AAV vectors. MAPT-targeted ZFPs reduced mouse and human tau by up to 99% with no detectable off-target gene regulation in primary and iPSC neurons. Intrahippocampal ZFP delivery to adult mice resulted in >90% tau reduction. Intravenous ZFP administration reduced tau levels by 50-70% across the entire brain. ZFP expression and mouse tau reduction were stable out to at least six months with no detectable off-targets, resulted in >80% lowering of CSF-tau, and reduced dystrophic neurites by 50% in APP/PS1 mice. Bilateral, real-time MRI-guided stereotaxis was used to deliver the ZFPs to the NHP hippocampus which was well tolerated and resulted in up to >80% tau lowering in the hippocampus and entorhinal cortex. ZFP levels were strongly correlated with tau reduction. The potency, efficacy, specificity and tolerability of ZFPs show they achieve permanent tau down-regulation for the treatment of human tauopathies.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526.

“TtAgo” is a prokaryotic Argonaute protein thought to be involved in gene silencing. TtAgo is derived from the bacteria Thermus thermophilus. (See, e.g., Swarts et al. (2014) Nature 507(7491):258-261, G. Sheng et al. (2014) Proc. Natl. Acad. Sci. U.S.A. 111(2):652). A “TtAgo system” is all the components required including, for example, guide DNAs for cleavage by a TtAgo enzyme. “Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

DNA-binding domains such as sgRNAs, zinc finger binding domains or TALE DNA binding domains can be “engineered” to bind to a predetermined nucleotide sequence, for example via design of a sgRNA that binds to a selected target site or by engineering (altering one or more amino acids) of the recognition helix region of a naturally occurring zinc finger protein or by engineering the RVDs of a TALE protein. Therefore, engineered zinc finger proteins or TALEs are proteins that are non-naturally occurring. Non-limiting examples of methods for engineering DNA-binding domains are design and selection. A “designed” zinc finger protein or TALE is a protein not occurring in nature whose design/composition results principally from rational criteria. Rational criteria for design include application of substitution rules and computerized algorithms for processing information in a database storing information of existing ZFP designs and binding data. A “selected” zinc finger protein or TALE is a protein not found in nature whose production results primarily from an empirical process such as phage display, interaction trap or hybrid selection. See, for example, U.S. Pat. Nos. 8,586,526; 6,140,081; 6,453,242; 6,746,838; 7,241,573; 6,866,997; 7,241,574; and 6,534,261; see also International Patent Publication No. WO 03/016496.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, where the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “multimerization domain” (also referred to as a “dimerization domain” or “protein interaction domain”) is a domain incorporated at the amino, carboxy or amino and carboxy terminal regions of a ZFP TF or TALE TF. These domains allow for multimerization of multiple ZFP TF or TALE TF units such that larger tracts of trinucleotide repeat domains become preferentially bound by multimerized ZFP TFs or TALE TFs relative to shorter tracts with wild-type numbers of lengths. Examples of multimerization domains include leucine zippers. Multimerization domains may also be regulated by small molecules where the multimerization domain assumes a proper conformation to allow for interaction with another multimerization domain only in the presence of a small molecule or external ligand. In this way, exogenous ligands can be used to regulate the activity of these domains.

A “gene” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALE protein as described herein. Thus, gene inactivation may be partial or complete.

A “genetic modulator” refers to any molecule that alters the expression and/or sequence of one or more genes. Non-limiting examples of genetic modulators include transcription factors (such as artificial transcription factors as described herein) that bind to the target gene and alter its expression and nucleases that modify the sequence of the target gene, which in turn alters its expression (e.g., inactivation of the target via insertions and/or deletions). Thus, a genetic modulator may be a genetic repressor (that represses and/or inactivates gene expression) or a genetic activator.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion molecule in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to upregulate gene expression. ZFPs fused to domains capable of regulating gene expression are collectively referred to as “ZFP-TFs” or “zinc finger transcription factors”, while TALEs fused to domains capable of regulating gene expression are collectively referred to as “TALE-TFs” or “TALE transcription factors.” When a fusion polypeptide in which a ZFP DNA-binding domain is fused to a cleavage domain (a “ZFN” or “zinc finger nuclease”), the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site. When a fusion polypeptide in which a TALE DNA-binding domain is fused to a cleavage domain (a “TALEN” or “TALE nuclease”), the TALE DNA-binding domain and the cleavage domain are in operative linkage if, in the fusion polypeptide, the TALE DNA-binding domain portion is able to bind its target site and/or its binding site, while the cleavage domain is able to cleave DNA in the vicinity of the target site. With respect to a fusion molecule in which a Cas DNA-binding domain (e.g., single guide RNA) is fused to an activation domain, the Cas DNA-binding domain and the activation domain are in operative linkage if, in the fusion polypeptide, the Cas DNA-binding domain portion is able to bind its target site and/or its binding site, while the activation domain is able to up-regulate gene expression. When a fusion polypeptide in which a Cas DNA-binding domain is fused to a cleavage domain, the Cas DNA-binding domain and the cleavage domain are in operative linkage if, in the fusion polypeptide, the Cas DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### Tau and Alzheimer's Disease

The tau protein is encoded by the MAPT gene which comprises 16 exons. Interestingly, exons 1, 4, 5, 7, 9, 11 and 12 and constitutively expressed whereas exons 2, 3, and 10 can be present in tau protein species derived from alternatively spliced variants, leading to the presence of six different tau protein isoforms in the adult brain. Tau binds to microtubules via 3 or 4 repeated tubulin-binding motifs in the C-terminal half of the protein, and is thought to stabilize the tubules where tau4R (4 tubulin binding motifs) is thought to interact more strongly with microtubules that tau3R. The ratio of 3R to 4R is generally stable but can be affected in pathological states. The tau form that interacts with microtubules is phosphorylated and it appears that hyperphosphorylation causes the tau to detach from microtubules. Hyperphosphoylated tau can be sequestered in the cell, which then leads to conformational changes in the protein and to aggregation. These aggregates may be the initial step in the formation of pathogenic neurofibrillary tangles (NFTs), however, hyperphosphorylated tau may be pathogenic in a soluble form as well as when present in the tangles (Bodea et al. (2016) J of Neurochem 138(Suppl 1):71-94). NFTs are restricted to the entorhinal cortex and medial temporal lobe in the early stages of AD, and by the time of severe clinical symptoms of the disease present, NFTs are widespread throughout the brain. Coincident with the presence of abundant NFTs, widespread distribution of amyloid plaques also occurs. In fact, it appears that the amyloid deposition in the cortex leads to an increase in the speed of tau propagation and the spread of NFT to distal regions of the brain. As tau tangles spread, there is a concomitant increase in neuronal loss (Pooler et al. (2015) Acta Neuropathol Commun 3:14, doi:10.1186/s40478-015-0199-x).

Tau has 95 amino acid residues that are capable of being phosphorylated, and several kinases have been identified that may be responsible for tau phosphorylation, which may be possible target candidates for new therapeutics, including glycogen synthase-3, cyclin-dependent kinase 5, members of the MAPK family, extracellular-regulated kinase, c-Jun N-terminal kinase and microtubule-affinity regulating kinase (Bodea (2016) ibid).

Amyloid β protein (Aβ) is the major constituent of senile plaques, which together with NFTs, are the hallmarks of a neuropathological confirmation of Alzheimer's Disease. Aβ is a peptide that has between 39 and 42 amino acid chains; the 42 amino acids form aggregates more avidly and is thought to be implicated in the pathogenesis of the disease and is the basis of the amyloid hypothesis (the proposal that accumulation of Aβ in the brain is the primary cause of AD, see review Hardy and Selkoe (2002) Science 297:353). Aβs are products of the proteolytic cleavage of amyloid precursor protein (APP), a ubiquitous, glycosylated, sulfated, and phosphorylated integral membrane protein (Sorrentino et al. (2014) FEBSLett 588:641-652). However, it is becoming clear that the pathogenesis leading to AD is extremely complex and that the pathogenesis of A3 accumulation may play a role in abnormal tau behavior (Ando et al. (2016) PLoS Genet 12(3):e1005917.

Reduction of tau in the brain has been shown to improve the pathology of AD. Regulated suppression of a tau transgene expression in a murine AD model demonstrated a reduction of transgene associated tau aggregates and a decrease in the concentration of hyperphosphorylated tau and NFT. In fact, this work also showed a loss in overall NFT, indicating that the accumulation of NFT may be reversible (Polydoro et al. (2013) J of Neurosci 33(33):13300-13311). Additionally, studies performed with an intracellular anti-tau antibody delivered via AAV directly through intrahippocampal administration demonstrated a reduction in insoluble tau species, NFT and a rescue of the hippocampal atrophy that is observed in the untreated mouse model (Liu et al. (2016) J Neurosci 36(49):12425-12435).

### DNA-Binding Domains

The methods described herein make use of compositions, for example tau-modulating transcription factors, comprising a DNA-binding domain that specifically binds to a target sequence in a tau (MATP) gene. Any polynucleotide or polypeptide DNA-binding domain can be used in the compositions and methods disclosed herein, for example DNA-binding proteins (e.g., ZFPs or TALEs) or DNA-binding polynucleotides (e.g., single guide RNAs). Thus, genetic modulators (repressors) of tau genes are described.

In certain embodiments, the tau-repressor, or DNA binding domain therein, comprises a zinc finger protein. Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

Tau target sites typically include at least one zinc finger but can include a plurality of zinc fingers (e.g., 2, 3, 4, 5, 6 or more fingers). Usually, the ZFPs include at least three fingers. Certain of the ZFPs include four, five or six fingers, while some ZFPs include 8, 9, 10, 11 or 12 fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains. In some embodiments, the fusion protein comprises two ZFP DNA binding domains linked together. These zinc finger proteins can thus comprise 8, 9, 10, 11, 12 or more fingers. In some embodiments, the two DNA binding domains are linked via an extendable flexible linker such that one DNA binding domain comprises 4, 5, or 6 zinc fingers and the second DNA binding domain comprises an additional 4, 5, or 5 zinc fingers. In some embodiments, the linker is a standard inter-finger linker such that the finger array comprises one DNA binding domain comprising 8, 9, 10, 11 or 12 or more fingers. In other embodiments, the linker is an atypical linker such as a flexible linker. The DNA binding domains are fused to at least one regulatory domain and can be thought of as a ‘ZFP-ZFP-TF’ architecture. Specific examples of these embodiments can be referred to as “ZFP-ZFP-KOX” which comprises two DNA binding domains linked with a flexible linker and fused to a KOX repressor and “ZFP-KOX-ZFP-KOX” where two ZFP-KOX fusion proteins are fused together via a linker.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

A ZFP can be operably associated (linked) to one or more transcriptional regulatory (e.g., repression domains) to form a ZF-TF (e.g., repressor). Methods and compositions can also be used to increase the specificity of a ZFP for its intended target relative to other unintended cleavage sites, known as off-target sites for example by mutations to the ZFP backbone as described in U.S. Patent Publication No. 2018/0087072. Thus, tau repressors described herein can comprise mutations in one or more of their DNA binding domain backbone regions and/or one or more mutations in their transcriptional regulatory domains. These ZFPs can include mutations to amino acid within the ZFP DNA binding domain (‘ZFP backbone’) that can interact non-specifically with phosphates on the DNA backbone, but they do not comprise changes in the DNA recognition helices. Thus, the invention includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In preferred embodiments, mutations at made at position (−5), (−9) and/or position (−14) relative to the DNA binding helix. In some embodiments, a zinc finger may comprise one or more mutations at (−5), (−9) and/or (−14). In further embodiments, one or more zinc finger in a multi-finger zinc finger protein may comprise mutations in (−5), (−9) and/or (−14). In some embodiments, the amino acids at (−5), (−9) and/or (−14) (e.g. an arginine (R) or lysine (K)) are mutated to an alanine (A), leucine (L), Ser (S), Asp (N), Glu (E), Tyr (Y) and/or glutamine (Q).

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22:1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 2007/0117128.

“Two handed” zinc finger proteins are those proteins in which two clusters of zinc finger DNA binding domains are separated by intervening amino acids so that the two zinc finger domains bind to two discontinuous target sites. An example of a two handed type of zinc finger binding protein is SIP1, where a cluster of four zinc fingers is located at the amino terminus of the protein and a cluster of three fingers is located at the carboxyl terminus (see Remade et al. (1999) EMBO Journal 18 (18):5073-5084). Each cluster of zinc fingers in these proteins is able to bind to a unique target sequence and the spacing between the two target sequences can comprise many nucleotides. Two-handed ZFPs may include a functional domain, for example fused to one or both of the ZFPs. Thus, it will be apparent that the functional domain may be attached to the exterior of one or both ZFPs or may be positioned between the ZFPs (attached to both ZFPs).

In certain embodiments, the DNA-binding domain comprises a naturally occurring or engineered (non-naturally occurring) TAL effector (TALE) DNA binding domain. See, e.g., U.S. Pat. No. 8,586,526, incorporated by reference in its entirety herein. In certain embodiments, the TALE DNA-binding protein comprises binds to 12, 13, 14, 15, 16, 17, 18, 19, 20 or more contiguous nucleotides of a tau target site as shown in U.S. Patent Publication No. 2018/0153921. The RVDs of the TALE DNA-binding protein that binds to a tau target site may be naturally occurring or non-naturally occurring RVDs. See, U.S. Pat. Nos. 8,586,526 and 9,458,205.

The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al. (2007) Science 318:648-651). These proteins contain a DNA binding domain and a transcriptional activation domain. One of the most well characterized TALEs is AvrBs3 from Xanthomonas campestgris pv. Vesicatoria (see Bonas et al. (1989)Mol Gen Genet 218: 127-136 and International Patent Publication No. WO 2010/079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack et al. (2006) J Plant Physiol 163(3):256-272). In addition, in the phytopathogenic bacteria Ralstonia solanacearum, two genes, designated brg11 and hpx17, have been found that are homologous to the AvrBs3 family of Xanthomonas in the R. solanacearum biovar 1 strain GMI1000 and in the biovar 4 strain RS1000 (See Heuer et al. (2007) Appl and EnvirMicro 73(13):4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove (2009) Science 326:1501 and Boch et al. (2009) Science 326:1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and NG binds to T. These DNA binding repeats have been assembled into proteins with new combinations and numbers of repeats, to make artificial transcription factors that are able to interact with new sequences. In addition, U.S. Pat. No. 8,586,526 and U.S. Patent Publication No. 2013/0196373, incorporated by reference in their entireties herein, describe TALEs with N-cap polypeptides, C-cap polypeptides (e.g., +63, +231 or +278) and/or novel (atypical) RVDs.

Exemplary TALEs are described in U.S. Pat. Nos. 8,586,526 and 9,458,205, incorporated by reference in their entireties.

In certain embodiments, the DNA binding domains include a dimerization and/or a multimerization domain, for example a coiled-coil (CC) and dimerizing zinc finger (DZ). See, U.S. Patent Publication No. 2013/0253040.

In still further embodiments, the DNA-binding domain comprises a single-guide RNA of a CRISPR/Cas system, for example sgRNAs as disclosed in 20150056705.

Compelling evidence has recently emerged for the existence of an RNA-mediated genome defense pathway in archaea and many bacteria that has been hypothesized to parallel the eukaryotic RNAi pathway (for reviews, see Godde and Bickerton (2006) J. Mol. Evol. 62:718-729; Lillestol et al. (2006) Archaea 2:59-72; Makarova et al. (2006) Biol. Direct 1:7; Sorek et al. (2008) Nat. Rev. Microbiol. 6:181-186). Known as the CRISPR-Cas system or prokaryotic RNAi (pRNAi), the pathway is proposed to arise from two evolutionarily and often physically linked gene loci: the CRISPR (clustered regularly interspaced short palindromic repeats) locus, which encodes RNA components of the system, and the cas (CRISPR-associated) locus, which encodes proteins (Jansen et al. (2002) Mol. Microbiol. 43:1565-1575; Makarova et al. (2002) Nucleic Acids Res. 30:482-496; Makarova et al. (2006) Biol. Direct 1:7; Haft et al. (2005) PLoS Comput. Biol. 1:e60). CRISPR loci in microbial hosts contain a combination of CRISPR-associated (Cas) genes as well as non-coding RNA elements capable of programming the specificity of the CRISPR-mediated nucleic acid cleavage. The individual Cas proteins do not share significant sequence similarity with protein components of the eukaryotic RNAi machinery, but have analogous predicted functions (e.g., RNA binding, nuclease, helicase, etc.) (Makarova et al. (2006) Biol. Direct 1:7). The CRISPR-associated (cas) genes are often associated with CRISPR repeat-spacer arrays. More than forty different Cas protein families have been described. Of these protein families, Cas1 appears to be ubiquitous among different CRISPR/Cas systems. Particular combinations of cas genes and repeat structures have been used to define 8 CRISPR subtypes (E. coli, Y. pest, N. meni, D. vulg, T neap, H. mari, A. pern, and M. tube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

The Type II CRISPR, initially described in S. pyogenes, is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences where processing occurs by a double strand-specific RNase III in the presence of the Cas9 protein. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In addition, the tracrRNA must also be present as it base pairs with the crRNA at its 3′ end, and this association triggers Cas9 activity. Finally, Cas9 mediates cleavage of target DNA to create a double-stranded break within the protospacer. Activity of the CRISPR/Cas system comprises three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system.

Type II CRISPR systems have been found in many different bacteria. BLAST searches on publically available genomes by Fonfara et al. (2013) Nuc Acid Res 42(4):2377-2590 found Cas9 orthologs in 347 species of bacteria. Additionally, this group demonstrated in vitro CRISPR/Cas cleavage of a DNA target using Cas9 orthologs from S. pyogenes, S. mutans, S. therophilus, C. jejuni, N. meningitides, P. multocida and F. novicida. Thus, the term “Cas9” refers to an RNA guided DNA nuclease comprising a DNA binding domain and two nuclease domains, where the gene encoding the Cas9 may be derived from any suitable bacteria.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The Cas 9 nuclease can be engineered such that only one of the nuclease domains is functional, creating a Cas nickase (see Jinek et al. (2012) Science 337:816). Nickases can be generated by specific mutation of amino acids in the catalytic domain of the enzyme, or by truncation of part or all of the domain such that it is no longer functional. Since Cas 9 comprises two nuclease domains, this approach may be taken on either domain. A double strand break can be achieved in the target DNA by the use of two such Cas 9 nickases. The nickases will each cleave one strand of the DNA and the use of two will create a double strand break.

The requirement of the crRNA-tracrRNA complex can be avoided by use of an engineered “single-guide RNA” (sgRNA) that comprises the hairpin normally formed by the annealing of the crRNA and the tracrRNA (see Jinek et al., ibid and Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam et al. (2013) Stem Cells and Development 22(4):595-610) and has been useful for zebrafish embryo genomic editing in vivo (see Hwang et al. (2013) Nature Biotechnology 31(3):227) with editing efficiencies similar to ZFNs and TALENs.

The primary products of the CRISPR loci appear to be short RNAs that contain the invader targeting sequences, and are termed guide RNAs or prokaryotic silencing RNAs (psiRNAs) based on their hypothesized role in the pathway (Makarova et al. (2006) Biol. Direct 1:7; Hale et al. (2008) RNA, 14:2572-2579). RNA analysis indicates that CRISPR locus transcripts are cleaved within the repeat sequences to release \tilde{ } 60- to 70-nt RNA intermediates that contain individual invader targeting sequences and flanking repeat fragments (Tang et al. (2002) Proc. Natl. Acad. Sci. 99:7536-7541; Tang et al. (2005) Mol. Microbiol. 55:469-481; Lillestol et al. (2006) Archaea 2:59-72; Brouns et al. (2008) Science 321:960-964; Hale et al. (2008) RNA, 14:2572-2579). In the archaeon Pyrococcus furiosus, these intermediate RNAs are further processed to abundant, stable \tilde{ } 35- to 45-nt mature psiRNAs (Hale et al. (2008) RNA, 14:2572-2579).

The requirement of the crRNA-tracrRNA complex can be avoided by use of an engineered “single-guide RNA” (sgRNA) that comprises the hairpin normally formed by the annealing of the crRNA and the tracrRNA (see Jinek et al. (2012) Science 337:816 and Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam, ibid) and has been useful for zebrafish embryo genomic editing in vivo (see Hwang et al. (2013) Nature Biotechnology 31 (3):227) with editing efficiencies similar to ZFNs and TALENs.

Chimeric or sgRNAs can be engineered to comprise a sequence complementary to any desired target. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In certain embodiments, the sgRNA comprises a sequence that binds to 12, 13, 14, 15, 16, 17, 18, 19, 20 or more contiguous nucleotides of a tau target site as shown in U.S. Publication No. 20180153921. In some embodiments, the RNAs comprise 22 bases of complementarity to a target and of the form G[n19], followed by a protospacer-adjacent motif (PAM) of the form NGG or NAG for use with a S. pyogenes CRISPR/Cas system. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence the conforms to the G[n20]GG formula. Along with the complementarity region, an sgRNA may comprise additional nucleotides to extend to tail region of the tracrRNA portion of the sgRNA (see Hsu et al. (2013) Nature Biotech doi:10.1038/nbt.2647). Tails may be of +67 to +85 nucleotides, or any number therebetween with a preferred length of +85 nucleotides. Truncated sgRNAs may also be used, “tru-gRNAs” (see Fu et al. (2014) Nature Biotech 32(3):279). In tru-gRNAs, the complementarity region is diminished to 17 or 18 nucleotides in length.

Further, alternative PAM sequences may also be utilized, where a PAM sequence can be NAG as an alternative to NGG (Hsu 2013, ibid) using a S. pyogenes Cas9. Additional PAM sequences may also include those lacking the initial G (Sander and Joung (2014) Nature Biotech 32(4):347). In addition to the S. pyogenes encoded Cas9 PAM sequences, other PAM sequences can be used that are specific for Cas9 proteins from other bacterial sources. For example, the PAM sequences shown below (adapted from Sander and Joung, ibid, and Esvelt et al. (2013) Nat Meth 10(11):1116) are specific for these Cas9 proteins:

| Species | PAM |
|---|---|
| S. pyogenes | NGG |
| S. pyogenes | NAG |
| S. mutans | NGG |
| S. thermophilius | NGGNG |
| S. thermophilius | NNAAAW |
| S. thermophilius | NNAGAA |
| S. thermophilius | NNNGATT |
| C. jejuni | NNNNACA |
| N. meningitides | NNNNGATT |
| P. multocida | GNNNCNNA |
| F. novicida | NG |

Thus, a suitable target sequence for use with a S. pyogenes CRISPR/Cas system can be chosen according to the following guideline: [n17, n18, n19, or n20](G/A)G. Alternatively the PAM sequence can follow the guideline G[n17, n18, n19, n20](G/A)G. For Cas9 proteins derived from non-S. pyogenes bacteria, the same guidelines may be used where the alternate PAMs are substituted in for the S. pyogenes PAM sequences.

Most preferred is to choose a target sequence with the highest likelihood of specificity that avoids potential off target sequences. These undesired off target sequences can be identified by considering the following attributes: i) similarity in the target sequence that is followed by a PAM sequence known to function with the Cas9 protein being utilized; ii) a similar target sequence with fewer than three mismatches from the desired target sequence; iii) a similar target sequence as in ii), where the mismatches are all located in the PAM distal region rather than the PAM proximal region (there is some evidence that nucleotides 1-5 immediately adjacent or proximal to the PAM, sometimes referred to as the ‘seed’ region (Wu et al. (2014) Nature Biotech doi:10.1038/nbt2889) are the most critical for recognition, so putative off target sites with mismatches located in the seed region may be the least likely be recognized by the sg RNA); and iv) a similar target sequence where the mismatches are not consecutively spaced or are spaced greater than four nucleotides apart (Hsu 2013, ibid). Thus, by performing an analysis of the number of potential off target sites in a genome for whichever CRIPSR/Cas system is being employed, using these criteria above, a suitable target sequence for the sgRNA may be identified.

In some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al. (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “‘Cas” includes both Cas9 and Cfp1 proteins. Thus, as used herein, a “CRISPR/Cas system” refers both CRISPR/Cas and/or CRISPR/Cfp1 systems, including both nuclease, nickase and/or transcription factor systems.

In some embodiments, other Cas proteins may be used. Some exemplary Cas proteins include Cas9, Cpf1 (also known as Cas12a), C2cl, C2c2 (also known as Cas13a), C2c3, Cas1, Cas2, Cas4, CasX and CasY; and include engineered and natural variants thereof (Burstein et al. (2017) Nature 542:237-241) for example HF1/spCas9 (Kleinstiver et al. (2016) Nature 529:490-495; Cebrian-Serrano and Davies (2017) Mamm Genome 28(7):247-261); split Cas9 systems (Zetsche et al. (2015) Nat Biotechnol 33(2):139-142), trans-spliced Cas9 based on an intein-extein system (Troung et al. (2015) Nucl Acid Res 43(13):6450-8); mini-SaCas9 (Ma et al. (2018) ACS Synth Biol 7(4):978-985). Thus, in the methods and compositions described herein, it is understood that the term “‘Cas” includes all Cas variant proteins, both natural and engineered. Thus, as used herein, a “CRISPR/Cas system” refers to any CRISPR/Cas system, including both nuclease, nickase and/or transcription factor systems.

In certain embodiments, the Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. In some aspects, a functional derivative may comprise a single biological property of a naturally occurring Cas protein. In other aspects, a function derivative may comprise a subset of biological properties of a naturally occurring Cas protein. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to specific genes (including safe harbor genes) are disclosed for example, in U.S. Patent Publication No. 2015/0056705.

Thus, the nuclease comprises a DNA-binding domain in that specifically binds to a target site in any gene into which it is desired to insert a donor (transgene) in combination with a nuclease domain that cleaves DNA.

### Tau Gene Modulators

The tau DNA-binding domains may be fused to or otherwise associate with any additional molecules (e.g., polypeptides) for use in the methods described herein. In certain embodiments, the methods employ fusion molecules comprising at least one DNA-binding molecule (e.g., ZFP, TALE or single guide RNA) and a heterologous regulatory (functional) domain (or functional fragment thereof).

In certain embodiments, the functional domain of the tau modulator comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases), protein degradation modifiers (de-ubiquitinases, ligases, degrons) and their associated factors and modifiers. See, e.g., U.S. Publication No. 2013/0253040, incorporated by reference in its entirety herein.

Suitable domains for achieving activation include the HSV VP16 activation domain (see, e.g., Hagmann et al. (1997) J. Virol. 71:5952-5962) nuclear hormone receptors (see, e.g., Torchia et al. (1998) Curr. Opin. Cell. Biol. 10:373-383); the p65 subunit of nuclear factor kappa B (Bitko & Barik (1998) J. Virol. 72:5610-5618 and Doyle & Hunt (1997) Neuroreport 8:2937-2942); Liu et al. (1998) Cancer Gene Ther. 5:3-28), or artificial chimeric functional domains such as VP64 (Beerli et al. (1998) Proc. Natl. Acad. Sci. USA 95:14623-33), and degron (Molinari et al. (1999) EMBO J. 18:6439-6447). Additional exemplary activation domains include, Oct 1, Oct-2A, Spl, AP-2, and CTF1 (Seipel et al. (1992) EMBO J. 11:4961-4968) as well as p300, CBP, PCAF, SRC1 PvALF, AtHD2A and ERF-2. See, for example, Robyr et al. (2000)Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

Exemplary repression domains that can be used to make tau repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rtt109 (Berndsen and Denu (2008) Curr Opin Struct Biol 18(6):682-689). In other instances the domain is a histone deacetylase (HDAC) such as the class I (HDAC-1, 2, 3, and 8), class II (HDAC IIA (HDAC-4, 5, 7 and 9), HDAC IIB (HDAC 6 and 10)), class IV (HDAC-11), class III (also known as sirtuins (SIRTs); SIRT1-7) (see Mottamal et al. (2015) Molecules 20(3):3898-3941). Another domain that is used in some embodiments is a histone phosphorylase or kinase, where examples include MSK1, MSK2, ATR, ATM, DNA-PK, Bub1, VprBP, IKK-α, PKCβ1, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dot1, PRMT 1/6, PRMT 5/7, PR-Set7 and Suv4-20h. Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al. (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

The fusion molecule may be formulated with a pharmaceutically acceptable carrier, as is known to those of skill in the art. See, for example, Remington's Pharmaceutical Sciences, 17th ed., 1985; and co-owned International Patent Publication No. WO 00/42219.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

In certain embodiments, the fusion molecule comprises a DNA-binding domain and a nuclease domain to create functional entities that are able to recognize their intended nucleic acid target through their engineered (ZFP or TALE or sgRNA) DNA binding domains and create nucleases (e.g., zinc finger nuclease or TALE nucleases or CRISPR/Cas nucleases) cause the DNA to be cut near the DNA binding site via the nuclease activity. This cleavage results in inactivation (repression) of a tau gene. Thus, tau repressors also include tau nucleases.

Thus, the methods and compositions described herein are broadly applicable and may involve any nuclease of interest. Non-limiting examples of nucleases include meganucleases, TALENs and zinc finger nucleases. The nuclease may comprise heterologous DNA-binding and cleavage domains (e.g., zinc finger nucleases; TALENs; meganuclease DNA-binding domains with heterologous cleavage domains, sgRNAs in association with nuclease domains) or, alternatively, the DNA-binding domain of a naturally-occurring nuclease may be altered to bind to a selected target site (e.g., a meganuclease that has been engineered to bind to site different than the cognate binding site).

The nuclease domain may be derived from any nuclease, for example any endonuclease or exonuclease. Non-limiting examples of suitable nuclease (cleavage) domains that may be fused to tau DNA-binding domains as described herein include domains from any restriction enzyme, for example a Type IIS Restriction Enzyme (e.g., FokI). In certain embodiments, the cleavage domains are cleavage half-domains that require dimerization for cleavage activity. See, e.g., U.S. Pat. Nos. 8,586,526; 8,409,861; and 7,888,121, incorporated by reference in their entireties herein. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing.

The nuclease domain may also be derived any meganuclease (homing endonuclease) domain with cleavage activity may also be used with the nucleases described herein, including but not limited to I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

In certain embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the meganuclease (e.g., Tev1) nuclease domain (see Beurdeley et al. (2013) Nat Comm 4(1762):1-8, DOI: 10.1038/ncomms2782).

In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al. (2013) Nucl Acid Res 42(4):2591-2601, doi: 10.1093/nar/gkt1224).

In addition, the nuclease domain of the meganuclease may also exhibit DNA-binding functionality. Any TALENs may be used in combination with additional TALENs (e.g., one or more TALENs (cTALENs or FokI-TALENs) with one or more mega-TALs) and/or ZFNs.

In addition, cleavage domains may include one or more alterations as compared to wild-type, for example for the formation of obligate heterodimers that reduce or eliminate off-target cleavage effects. See, e.g., U.S. Pat. Nos. 7,914,796; 8,034,598; and 8,623,618, incorporated by reference in their entireties herein.

Nucleases as described herein may generate double- or single-stranded breaks in a double-stranded target (e.g., gene). The generation of single-stranded breaks (“nicks”) is described, for example in U.S. Pat. Nos. 8,703,489 and 9,200,266, incorporated herein by reference which describes how mutation of the catalytic domain of one of the nucleases domains results in a nickase.

Thus, a nuclease (cleavage) domain or cleavage half-domain can be any portion of a protein that retains cleavage activity, or that retains the ability to multimerize (e.g., dimerize) to form a functional cleavage domain.

Alternatively, nucleases may be assembled in vivo at the nucleic acid target site using so-called “split-enzyme” technology (see e.g. U.S. Patent Publication No. 2009/0068164). Components of such split enzymes may be expressed either on separate expression constructs, or can be linked in one open reading frame where the individual components are separated, for example, by a self-cleaving 2A peptide (e.g., T2A) or IRES sequence. Components may be individual zinc finger binding domains or domains of a meganuclease nucleic acid binding domain.

Nucleases can be screened for activity prior to use, for example in a yeast-based chromosomal system as described in U.S. Patent Publication No. 2009/0111119. Nuclease expression constructs can be readily designed using methods known in the art.

Expression of the fusion proteins (or component thereof) may be under the control of a constitutive promoter or an inducible promoter, for example the galactokinase promoter which is activated (de-repressed) in the presence of raffinose and/or galactose and repressed in presence of glucose. Non-limiting examples of preferred promoters include the neural specific promoters NSE, Synapsin, CAMKiia and MECPs. Non-limiting examples of ubiquitous promoters include CAS and Ubc. Further embodiments include the use of self-regulating promoters (via the inclusion of high affinity binding sites for the tau DNA-binding domain) as described in U.S. Patent Publication No. 2015/0267205).

In certain embodiments, the tau modulators for use in a subject comprise ZFPs designated 57890, 65918 and/or 57930. In certain embodiments, two more such tau modulators that provide synergistic effects as compared to a single ZFP tau repressor (e.g., 57890 and 65918 ZFP repressors; 57890 and 57930 ZFP repressors; 65918 and 57930; 57890, 65918 and 57930) are provided to the subject for repression of tau and treatment and/or prevention of a tauopathy such as AD, including the amelioration of symptoms of the tauopathy.

### Delivery

The proteins and/or polynucleotides (e.g., tau modulators) and compositions comprising the proteins and/or polynucleotides described herein may be delivered to a target cell by any suitable means including, for example, by injection of proteins, via mRNA and/or using an expression construct (e.g., plasmid, lentiviral vector, AAV vector, Ad vector, etc.). In preferred embodiments, the repressor is delivered using an AAV vector, including but not limited to AAV9 (see U.S. Pat. No. 7,198,951), an AAV vector as described in U.S. Pat. No. 9,585,971.

Methods of delivering proteins comprising zinc finger proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 8,586,526; 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences. Thus, when one or more tau modulators (e.g., repressors) are introduced into the cell, the sequences encoding the protein components and/or polynucleotide components may be carried on the same vector or on different vectors. When multiple vectors are used, each vector may comprise a sequence encoding one or multiple tau modulators (e.g., repressors) or components thereof. In preferred embodiments, the vector system is an AAV vector, for example AAV9 or an AAV variant described in U.S. Pat. No. 9,585,971 or U.S. Patent Publication No. 2017/0119906.

Conventional viral and non-viral based gene transfer methods can be used to introduce nucleic acids encoding engineered tau modulators in cells (e.g., mammalian cells) and target tissues. Such methods can also be used to administer nucleic acids encoding such repressors (or components thereof) to cells in vitro. In certain embodiments, nucleic acids encoding the repressors are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6(10):1149-1154 (1988); Vigne (1995) Restorative Neurology and Neuroscience 8:35-36; Kremer & Perricaudet (1995) British Medical Bulletin 51(1):31-44; Haddada et al., in Current Topics in Microbiology and Immunology Doerfler and Bohm (eds.) (1995); and Yu et al. (1994) Gene Therapy 1:13-26.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Maryland), BTX Molecular Delivery Systems (Holliston, MA) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™ and Lipofectamine™ RNAiMAX). Cationic and neutral lipids that are suitable for efficient receptor-recognition lipofection of polynucleotides include those of Felgner, International Patent Publication Nos. WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal (1995) Science 270:404-410 (1995); Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al. (2009) Nature Biotechnology 27(7):643).

The use of RNA or DNA viral based systems for the delivery of nucleic acids encoding engineered ZFPs, TALEs or CRISPR/Cas systems take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs, TALEs or CRISPR/Cas systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon mouse leukemia virus (MuLV), gibbon ape leukemia virus (GaLV), Simian Immunodeficiency virus (SIV), human immunodeficiency virus (HIV), and combinations thereof (see, e.g., Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; International Patent Publication No. WO 1994/026877).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al. (1987) Virology 160:38-47; U.S. Pat. No. 4,797,368; International Patent Publication No. WO 93/24641; Kotin (1994) Human Gene Therapy 5:793-801; Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin et al. (1984) Mol. Cell. Biol. 4:2072-2081; Hermonat & Muzyczka (1984) PNAS 81:6466-6470; and Samulski et al. (1989) J. Virol. 63:03822-3828.

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al. (1995) Blood 85:3048-305; Kohn et al. (1995) Nat. Med. 1:1017-102; Malech et al. (1997) PNAS 94(22):12133-12138). PA317/pLASN was the first therapeutic vector used in a gene therapy trial. (Blaese et al. (1995) Science 270:475-480). Transduction efficiencies of 50% or greater have been observed for MFG-S packaged vectors. (Ellem et al. (1997) Immunol Immunother. 44(1):10-20; Dranoff et al. (1997) Hum. Gene Ther. 1:111-2.

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al. (1998) Lancet 351(9117):1702-3, Kearns et al. (1996) Gene Ther. 9:748-55). Other AAV serotypes, including AAV1, AAV3, AAV4, AAV5, AAV6, AAV8, AAV 8.2, AAV9, and AAV rh10 and pseudotyped AAV such as AAV2/8, AAV2/5, AAV2/9 and AAV2/6 can also be used in accordance with the present invention. Novel AAV serotypes capable of crossing the blood-brain barrier can also be used in accordance with the present invention (see e.g. U.S. Pat. No. 9,585,971). In preferred embodiments, an AAV9 vector (including variants and pseudotypes of AAV9) is used.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al. (1998) Hum. Gene Ther. 7:1083-9). Additional examples of the use of adenovirus vectors for gene transfer in clinical trials include Rosenecker et al. (1996) Infection 24(1):5-10; Sterman et al. (1998) Hum. Gene Ther. 9(7):1083-1089; Welsh et al. (1995) Hum. Gene Ther. 2:205-18; Alvarez et al. (1997) Hum. Gene Ther. 5:597-613; Topf et al. (1998) Gene Ther. 5:507-513; Sterman et al. (1998) Hum. Gene Ther. 7:1083-1089.

Packaging cells are used to form virus particles that are capable of infecting a host cell. Such cells include 293 cells, which package adenovirus, and W2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751, reported that Moloney mouse leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, intrathecal, intracisternal, intracerebroventricular, or intracranial infusion, including direct injection into the brain including into any region of the brain such as the hippocampus, cortex, striatum, etc.) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

In certain embodiments, the compositions as described herein (e.g., polynucleotides and/or proteins) are delivered directly in vivo. The compositions (cells, polynucleotides and/or proteins) may be administered directly into the central nervous system (CNS), including but not limited to direct injection into the brain or spinal cord. One or more areas of the brain may be targeted, including but not limited to, the hippocampus, the substantia nigra, the nucleus basalis of Meynert (NBM), the striatum and/or the cortex. Alternatively or in addition to CNS delivery, the compositions may be administered systemically (e.g., intravenous, intraperitoneal, intracardial, intramuscular, subdermal, intrathecal, intracisternal, intracerebroventricular and/or intracranial infusion). Methods and compositions for delivery of compositions as described herein directly to a subject (including directly into the CNS) include but are not limited to direct injection (e.g., stereotactic injection) via needle assemblies. Such methods are described, for example, in U.S. Pat. Nos. 7,837,668 and 8,092,429, relating to delivery of compositions (including expression vectors) to the brain and U.S. Patent Publication No. 2006/0239966, incorporated herein by reference in their entireties.

The effective amount to be administered will vary from patient to patient and according to the mode of administration and site of administration. Accordingly, effective amounts are best determined by the physician administering the compositions and appropriate dosages can be determined readily by one of ordinary skill in the art. After allowing sufficient time for integration and expression (typically 4-15 days, for example), analysis of the serum or other tissue levels of the therapeutic polypeptide and comparison to the initial level prior to administration will determine whether the amount being administered is too low, within the right range or too high. Suitable regimes for initial and subsequent administrations are also variable, but are typified by an initial administration followed by subsequent administrations if necessary. Subsequent administrations may be administered at variable intervals, ranging from daily to annually to every several years. In certain embodiments,

To deliver ZFPs using adeno-associated viral (AAV) vectors directly to the human brain, a dose range of about 1×10¹⁰-5×10¹⁵ (or any value therebetween) vector genome per striatum can be applied. As noted, dosages may be varied for other brain structures and for different delivery protocols. Methods of delivering AAV vectors directly to the brain are known in the art. See, e.g., U.S. Pat. Nos. 9,089,667; 9,050,299; 8,337,458; 8,309,355; 7,182,944; 6,953,575; and 6,309,634.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with at least one tau modulator (e.g., repressor) or component thereof and re-infused back into the subject organism (e.g., patient). In a preferred embodiment, one or more nucleic acids of the tau modulator (e.g., repressor) are delivered using AAV9. In other embodiments, one or more nucleic acids of the tau modulator (e.g., repressor) are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein in their entireties. Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al. (1992) J. Exp. Med. 176:1693-1702).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells) (see Inaba et al. (1992) J. Exp. Med. 176:1693-1702).

Stem cells that have been modified may also be used in some embodiments. For example, neuronal stem cells that have been made resistant to apoptosis may be used as therapeutic compositions where the stem cells also contain the ZFP TFs of the invention. Resistance to apoptosis may come about, for example, by knocking out BAX and/or BAK using BAX- or BAK-specific TALENs or ZFNs (see, U.S. Pat. No. 8,597,912) in the stem cells, or those that are disrupted in a caspase, again using caspase-6 specific ZFNs for example. These cells can be transfected with the ZFP TFs or TALE TFs that are known to regulate a tau gene.

Vectors (e.g., retroviruses, adenoviruses, liposomes, etc.) containing therapeutic ZFP nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34+ cells, include adenovirus Type 35.

Vectors suitable for introduction of transgenes into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells. Suitable cell lines for protein expression are known to those of skill in the art and include, but are not limited to COS, CHO (e.g., CHO—S, CHO-K1, CHO-DG44, CHO-DUXB11), VERO, MDCK, W138, V79, B14AF28-G3, BHK, HaK, NS0, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pischia and Schizosaccharomyces. Progeny, variants and derivatives of these cell lines can also be used. In a preferred embodiment, the methods and composition are delivered directly to a brain cell, for example in the striatum.

### Models of CNS Disorders

Studies of CNS disorders can be carried out in animal model systems such as non-human primates (e.g., Parkinson's Disease (Johnston and Fox (2015) Curr Top Behav Neurosci 22: 221-35); Amyotrophic lateral sclerosis (Jackson et al. (2015) J. Med Primatol: 44(2):66-75), Huntington's Disease (Yang et al. (2008) Nature 453(7197):921-4); Alzheimer's Disease (Park et al. (2015) Int J Mol Sci 16(2):2386-402); Seizure (Hsiao et al. (2016) E Bio Med 9:257-77), canines (e.g. MPS VII (Gurda et al. (2016) Mol Ther 24(2):206-216); Alzheimer's Disease (Schutt et al. (2016) J Alzheimers Dis 52(2):433-49); Seizure (Varatharajah et al. (2017) Int J Neural Syst 27(1):1650046) and mice (e.g. Seizure (Kadiyala et al. (2015) Epilepsy Res 109:183-96); Alzheimer's Disease (Li et al. (2015) J Alzheimers Dis Parkin 5(3) doi 10:4172/2161-0460), (review: Webster et al. (2014) Front Genet 5(99):1-23, doi:10.3389f/gene.2014.00088). These models may be used even when there is no animal model that completely recapitulates a CNS disease as they may be useful for investigating specific symptom sets of a disease. The models may be helpful in determining efficacy and safety profiles of a therapeutic methods and compositions (genetic repressors) described herein.

### Applications

Tau modulators (e.g., tau repressors) as described herein comprising MAPT-binding molecules (e.g., ZFPs, TALEs, CRISPR/Cas systems, Ttago, etc.) as described herein, and the nucleic acids encoding them, can be used for a variety of applications. These applications include therapeutic methods in which a MAPT-binding molecule (including a nucleic acid encoding a DNA-binding protein) is administered to a subject using a viral (e.g., AAV) or non-viral vector and used to modulate the expression of a target gene within the subject. The modulation can be in the form of repression, for example, repression of tau expression that is contributing to an AD disease state. Alternatively, the modulation can be in the form of activation when activation of expression or increased expression of an endogenous cellular gene can ameliorate a diseased state. In still further embodiments, the modulation can be repression via cleavage (e.g., by one or more nucleases), for example, for inactivation of a MAPT gene. As noted above, for such applications, the MAPT-binding molecules, or more typically, nucleic acids encoding them are formulated with a pharmaceutically acceptable carrier as a pharmaceutical composition.

The MAPT-binding molecules, or vectors encoding them, alone or in combination with other suitable components (e.g. liposomes, nanoparticles or other components known in the art), can be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically, retro-orbitally (RO), intracranially (e.g., to any area of the brain including but not limited to the hippocampus and/or cortex), intracisternally or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient should be sufficient to effect a beneficial therapeutic response in the patient over time. The dose is determined by the efficacy and K_{d} of the particular MAPT-binding molecule employed, the target cell, and the condition of the patient, as well as the body weight or surface area of the patient to be treated. The size of the dose also is determined by the existence, nature, and extent of any adverse side-effects that accompany the administration of a particular compound or vector in a particular patient

The following Examples relate to exemplary embodiments of the present disclosure in which the MAPT-modulator comprises a zinc finger protein. It will be appreciated that this is for purposes of exemplification only and that other MAPT-modulators (e.g., repressors) can be used, including, but not limited to, TALE-TFs, a CRISPR/Cas system, additional ZFPs, ZFNs, TALENs, additional CRISPR/Cas systems, homing endonucleases (meganucleases) with engineered DNA-binding domains. It will be apparent that these modulators can be readily obtained using methods known to the skilled artisan to bind to the target sites as exemplified below. Similarly, the following Examples relate to exemplary embodiments in which the delivery vehicle is any AAV vector but it will apparent that any viral (Ad, LV, etc.) or non-viral (plasmid, mRNA, etc.) can be used to deliver the tau repressors described herein.

### EXAMPLES

### Example 1: In Vivo MAPT Repression

Zinc finger proteins specific for MAPT (tau) target sites as described in U.S. Publication No. 2018/0153921 were used as follows:

TABLE 1
MAPT-specific designs

| | Design | | | | | |
|---|---|---|---|---|---|---|
| SBS #, Target | F1 | F2 | F3 | F4 | F5 | F6 |
| SBS# 57890 | LRHHLT | RRFTLS | RSDVL | KHSTR | RSDVL | RLYTL |
| tgGTGCTGGA | R | K | SE | RV | SE | HK |
| GCTGGTGGGT | (SEQ ID | (SEQ ID | (SEQ ID | (SEQ ID | (SEQ ID | (SEQ ID |
| ggcggaga (SEQ | NO: 3) | NO: 4) | NO: 5) | NO: 6) | NO: 5) | NO: 7) |
| ID NO: 1) | | | | | | |
| SBS# 57930 | DRSHLT | LKQHL | RSAHL | TSGHL | QSGNL | QSGDL |
| cgGCAGAAGG | R | TR | SR | SR | AR | TR |
| TGGGcGGTGG | (SEQ ID | (SEQ ID | (SEQ ID | (SEQ ID | (SEQ ID | (SEQ ID |
| Cggcggcg (SEQ | NO: 8) | NO: 9) | NO: 10) | NO: 11) | NO: 12) | NO: 13) |
| ID NO: 2) | | | | | | |
| [Parent] | | | | | | |
| SBS# 65918 | DRSHLT | LKQHL | RSAHL | TSGHL | QSGNL | QSGDL |
| cgGCAGAAGG | R | TR | SR | SR | AR | TR |
| TGGGcGGTGG | (SEQ ID | (SEQ ID | (SEQ ID | (SEQ ID | (SEQ ID | (SEQ ID |
| Cggcggcg (SEQ | NO: 8) | NO: 9) | NO: 10) | NO: 11) | NO: 12) | NO: 13) |
| ID NO: 2) | | | | | | |
| 65918 Phos | Qm5 | none | none | none | Qm5 | none |
| contact mutants | | | | | | |

All ZFPs described herein were operably linked to a KRAB repression domain to form ZFP-TFs and all repressed MAPT expression.

Primate tau-specific ZFP-TFs are tested in cynomolgus monkeys (M. fascicularis) to observe repression of tau expression in a primate (non-human primate (NHIP) model). Cynomolgus monkeys are housed in stainless steel cages equipped with an automatic watering system. The study complies with all applicable sections of the current version of the Final Rules of the Animal Welfare Act Regulations (Code of Federal Regulations, Title 9) and the Guide for the Care and Use of Laboratory Animals, Institute of Laboratory Animal Resources, Commission on Life Sciences, National Research Council, 8th edition.

The ZFP-TF repressors of Table 1 were cloned into an AAV vector (AAV2/9, or variants thereof) with the SYN1 promoter or CMV promoter, essentially as described in U.S. Publication No. 20180153921. The AAV vectors that were used included: a vector with a SYN1 promoter driving expression of repressors comprising 65918 and 57890 linked by a T2A peptide (SYN918-890); a vector with a CMV promoter driving expression of repressors comprising 65918 and 57890 linked by a T2A peptide (CMV918-890); a vector with a SYN1 promoter driving expression of a repressor comprising 57930 (SYN930); a vector with a SYN1 promoter driving expression of a repressor comprising 57890 (SYN890); and a vector with a SYN1 promoter driving expression of a repressor comprising 65918 (SYN918).

Fifteen NHP subjects were treated as shown in the following Table:

TABLE 2

| | | rAAV |
|---|---|---|
| Cohort | AAV-promoter ZFP | vg/hemisphere |
| NHP01 | Vehicle only | 0 |
| NHP02 | Vehicle only | 0 |
| NHP03 | Vehicle only | 0 |
| NHP04 | Synapsin-65918 and 57890 | 6E11 |
| NHP05 | Synapsin-65918 and 57890 | 6E11 |
| NHP06 | Synapsin-65918 and 57890 | 6E11 |
| NHP07 | CMV-65918 and 57890 | 6E11 |
| NHP08 | CMV-65918 and 57890 | 6E11 |
| NHP09 | Synapsin-57930 | 6E11 |
| NHP10 | Synapsin-57930 | 6E11 |
| NHP11 | Synapsin-57890 | 6E11 |
| NHP12 | Synapsin-57890 | 6E11 |
| NHP13 | Synapsin-57890 | 6E11 |
| NHP14 | Synapsin-65918 | 6E11 |
| NHP15 | Synapsin-65918 | 6E11 |

In the experiment, AAV9 vectors comprising a hSYN1 or CMV driven ZFP TF are delivered at 6E11 vg/hemisphere to the left and 6E11 vg/hemisphere to the right hemisphere. Animals received a single dose of test article in a volume of 60 μL in the left and a single dose of 60 μL in the right hemisphere. For all test articles, the dose concentration was 1E13 vg/mL.

After 28 days, the animals were sacrificed, and the brains were removed and placed in a coronal brain matrix in ice-cold PBS. Brains were sliced at a 3 mm coronal slice thickness (divided into approximately 17 slices). Some brain slices (right and left hemisphere) were stored in 10% neutral-buffered formalin for histopathology and in situ hybridization analyses. All other brain slices (right and left hemisphere) were placed in RNAlater (Qiagen) and refrigerated for approximately 24 hours, after which 2-3 mm diameter punches were collected according to a predefined brain template. Punches were processed for qRT-PCR and biodistribution analysis. Additionally, CSF was collected for tau protein analysis.

Slices comprising the hippocampus and entorhinal cortex regions were used to analyze mRNA expression levels of tau, ZFP, glial and neuronal cell markers, and housekeeping genes via qRT-PCR. The results show that the ZFP-TFs were delivered by AAV to the hippocampal region leading to reduction in tau expression.

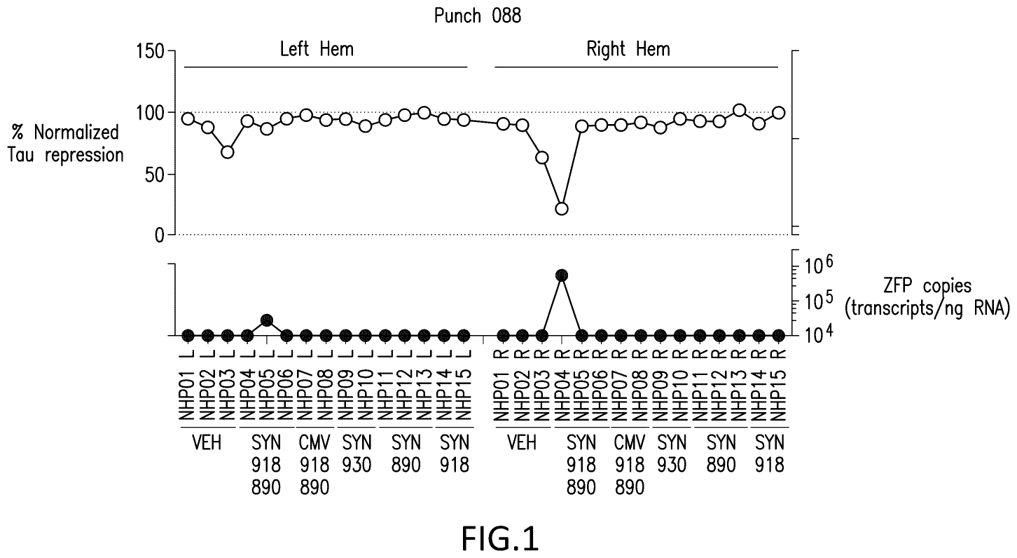

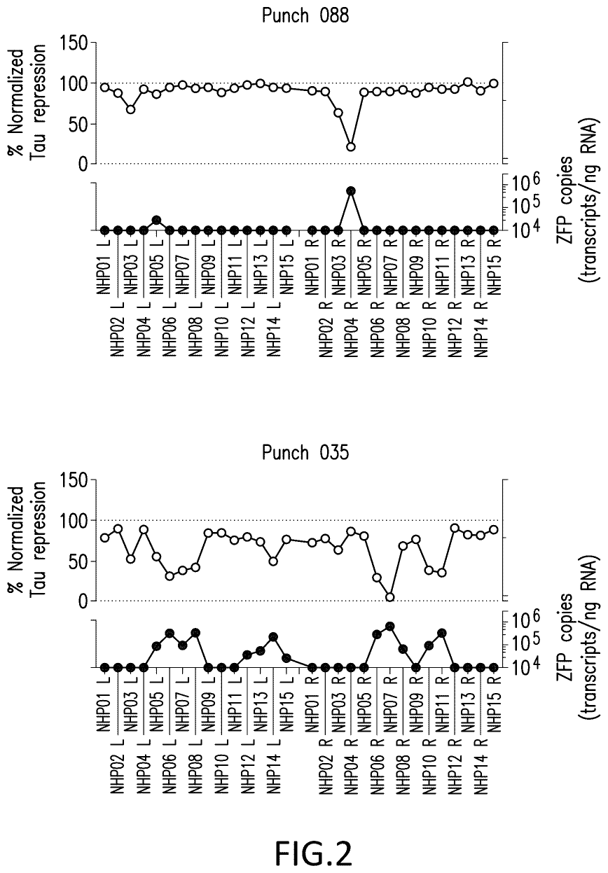

FIG. 1 and FIG. 2 show exemplary results of tau repression and ZFP mRNA levels (copies/ng mRNA) of hippocampal punches 088 and 035 using the indicated vectors in the indicated subjects.

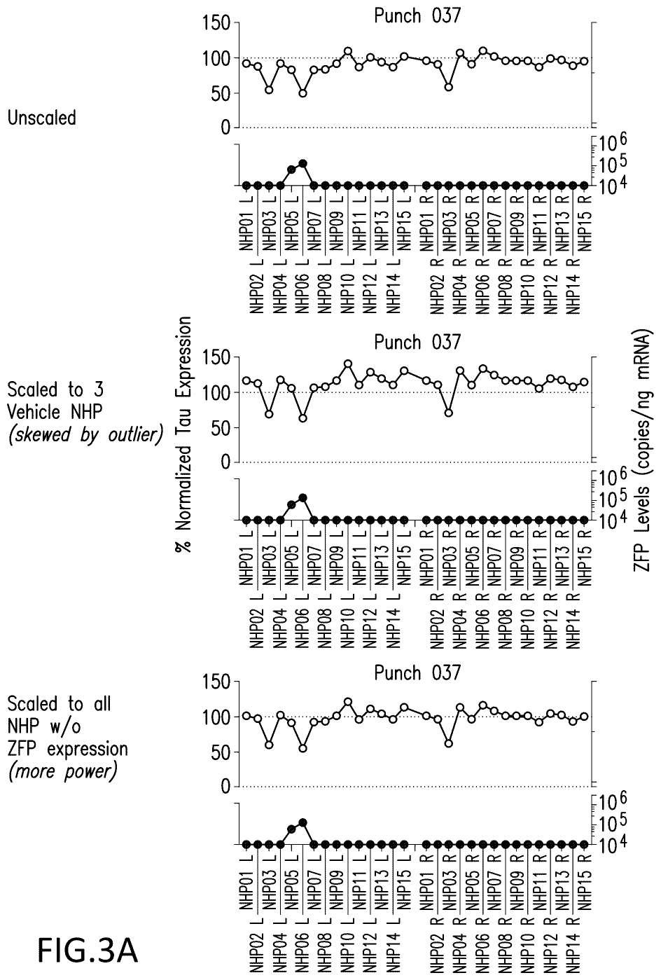

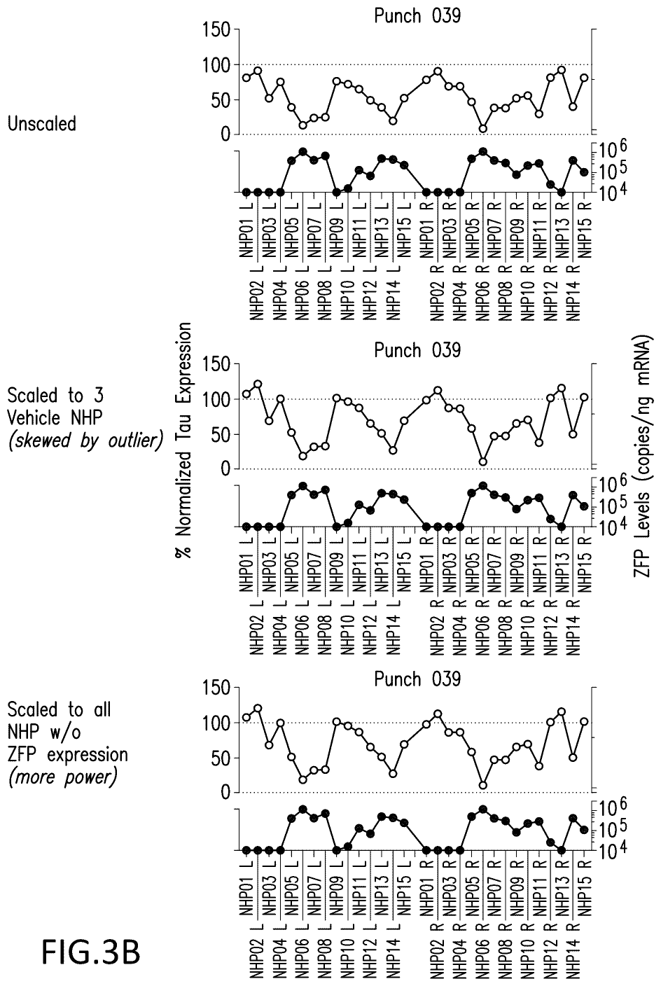

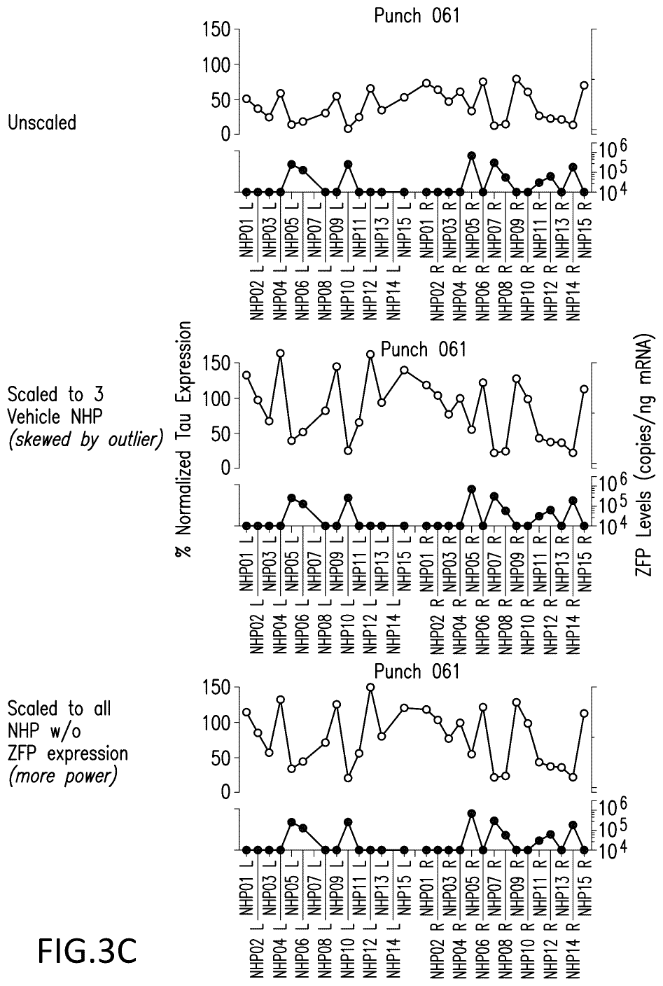

FIG. 3A through FIG. 3C show results from the indicated punch samples and three methods evaluated to establish baseline tau levels for a given punch, including no scaling (top graphs), scaling to the average of the three vehicle-treated animals (middle graphs), or scaling to the average tau expression of the vehicle-treated animals and ZFP-treated animals without detectable ZFP expression (bottom graphs). As shown, scaling by either method better approximates baseline levels across all 15 animals, with the third method (scaling to NHPs without ZFP expression) being a somewhat better representative of the tau baseline across the 15 animals in the study in some cases (e.g., Punch 037).

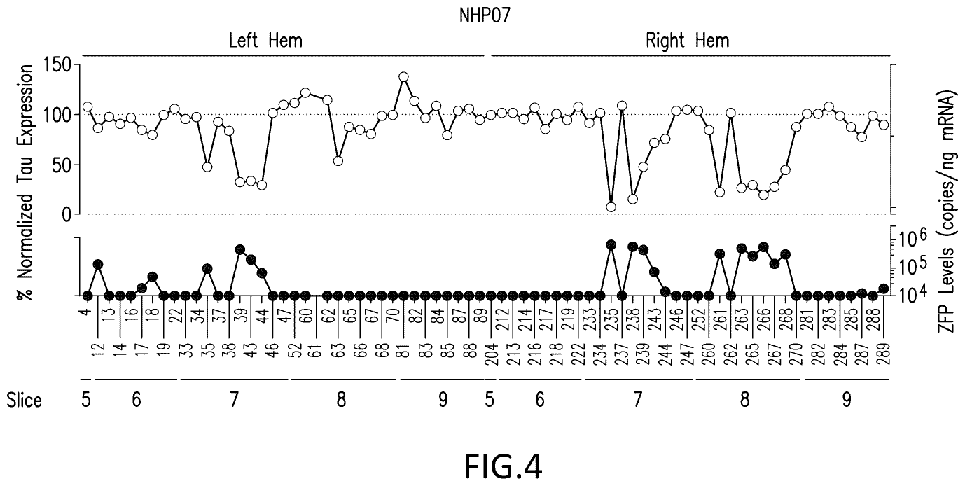

FIG. 4 shows tau modulation and ZFP levels in subject NHP07 which was treated with AAV CMV918-890. For this analysis, normalized tau expression was scaled to the average of the tau levels measured from vehicle-treated animals and ZFP-treated animals without detectable ZFP expression for each punch. Data from each punch for NHP07 was then extracted and grouped according to brain section analyzed. As shown, in NHP07 showed tau repression in certain brain samples.

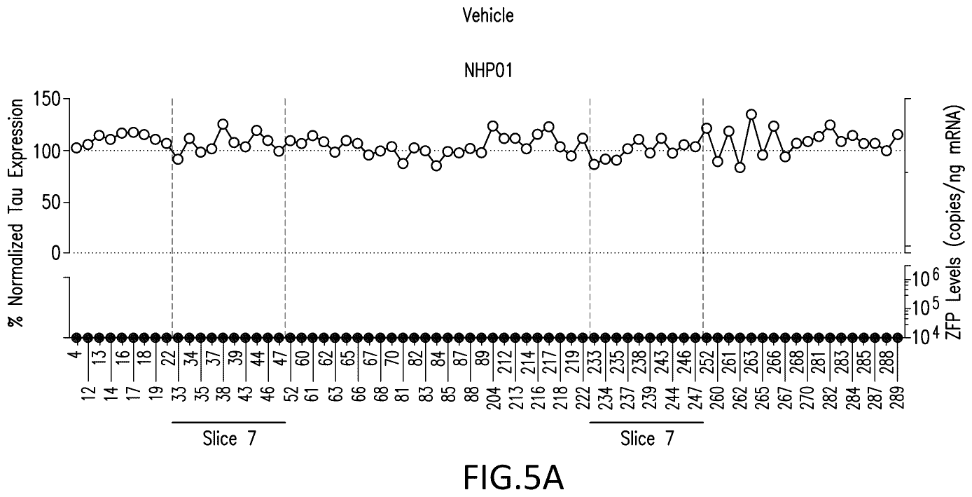

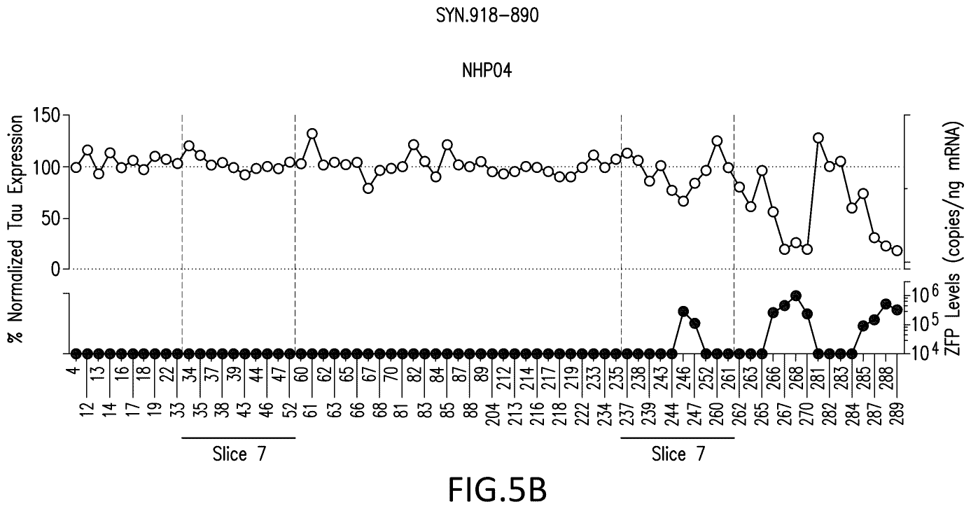

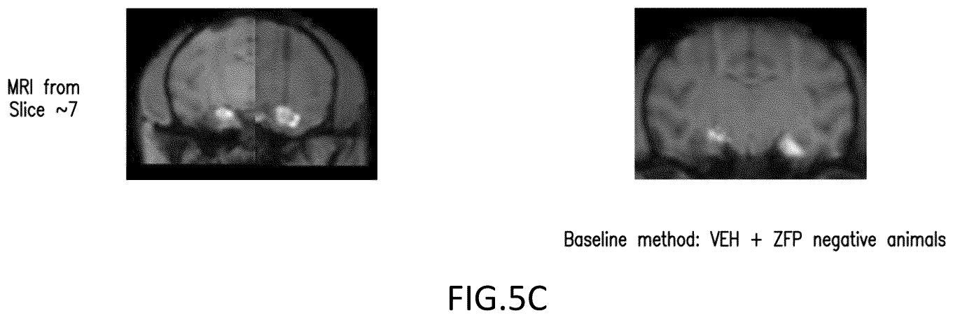

FIG. 5A and FIG. 5B show a comparison of a tau expression and ZFP levels in a control subject (Vehicle, NHP01) and a subject treated with AAV SYN918-890 (NP04) and FIG. 5C shows MRI results from slice 7. For this analysis, normalized tau expression was scaled to the average of the tau levels measured from vehicle-treated animals and ZFP-treated animals without detectable ZFP expression for each punch.

As shown, the vehicle-treated NHP had no detectable tau reduction or ZFP expression. In contrast, NHP04 showed detectable ZFP expression in slice 7 in the right hemisphere which also correlated with two medial hippocampal punches. Increased ZFP coverage and tau reduction was observed for Sections 8 and 9 in the right hemisphere which correlated well with MRI data from those levels of the brain.

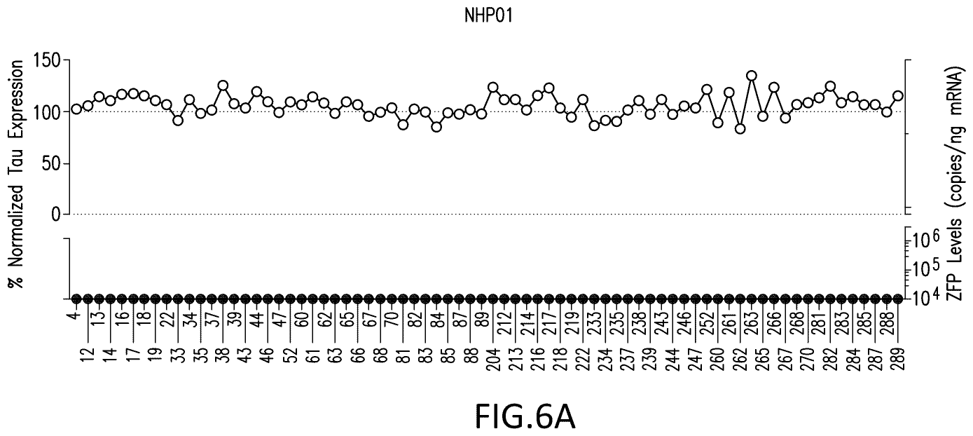

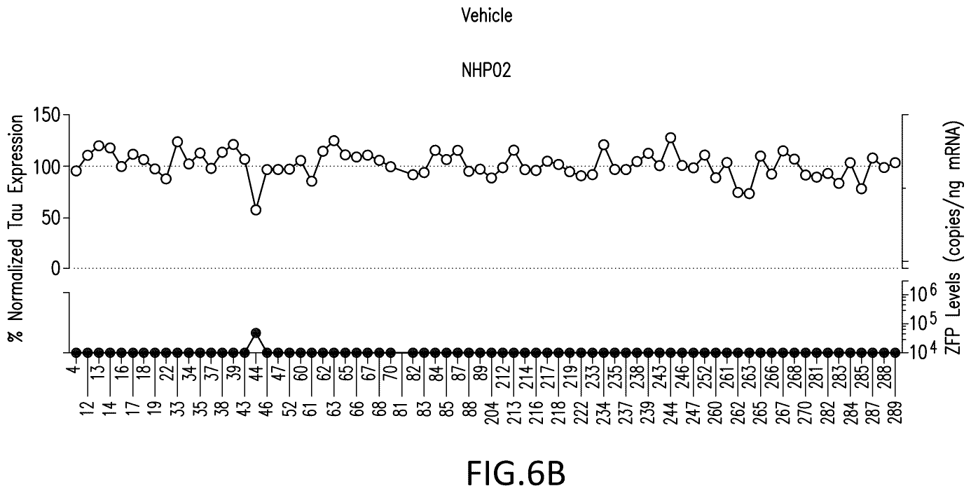

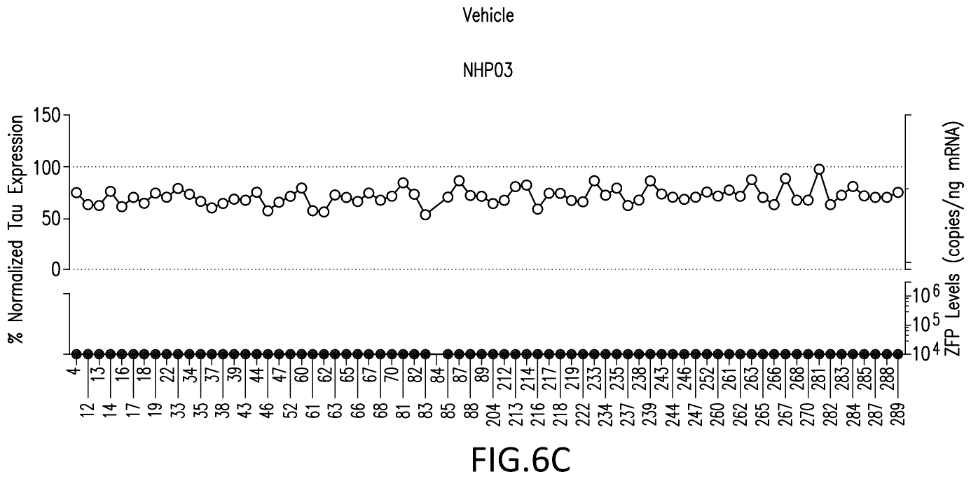

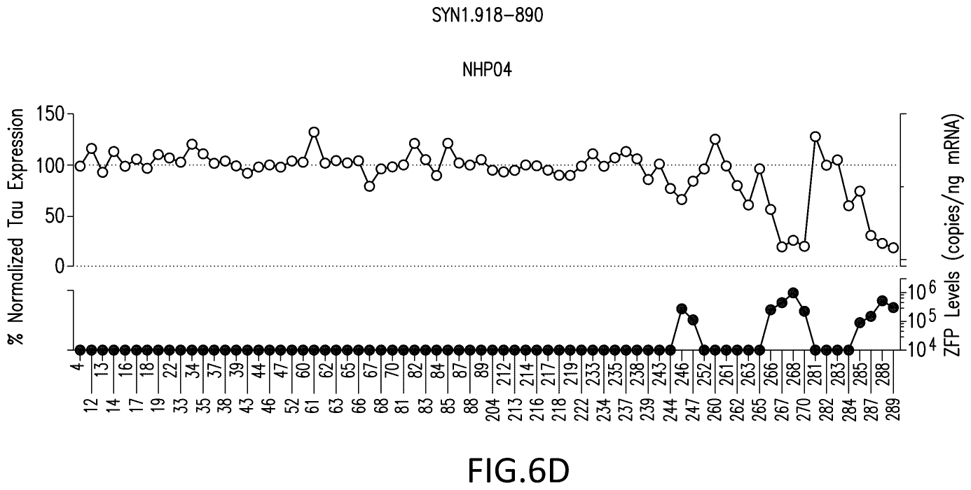

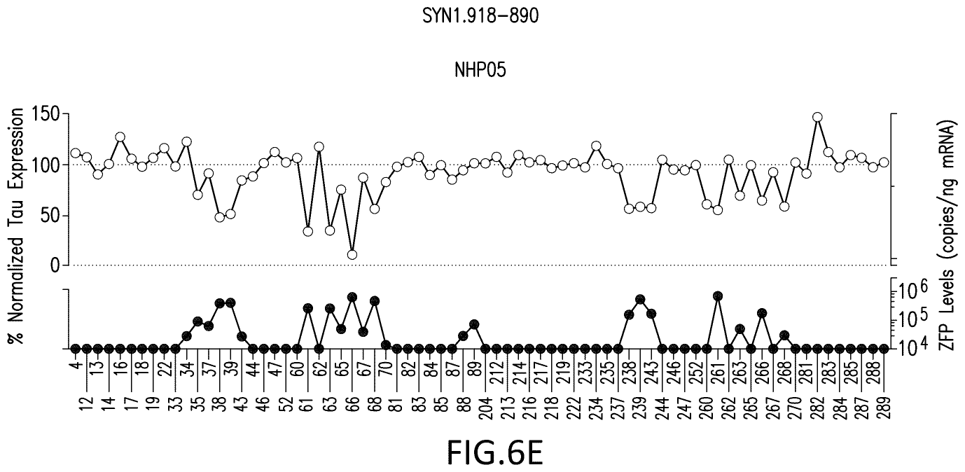

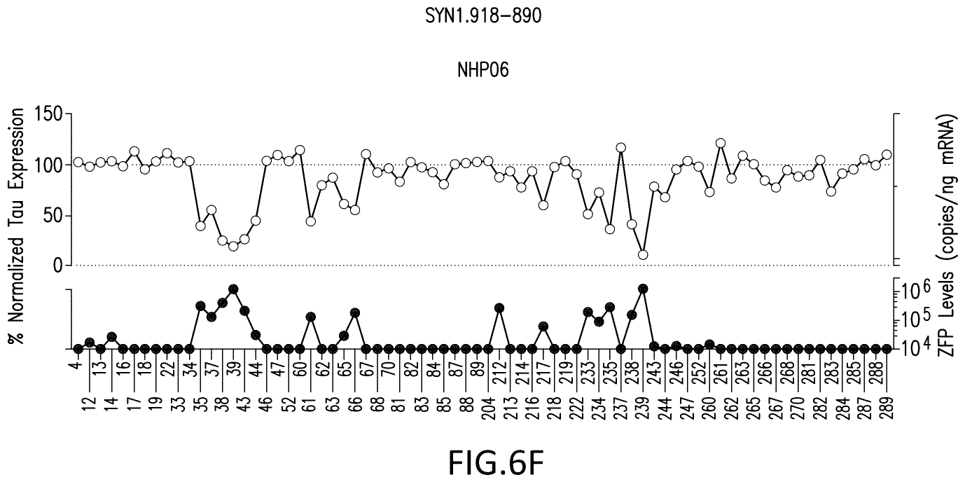

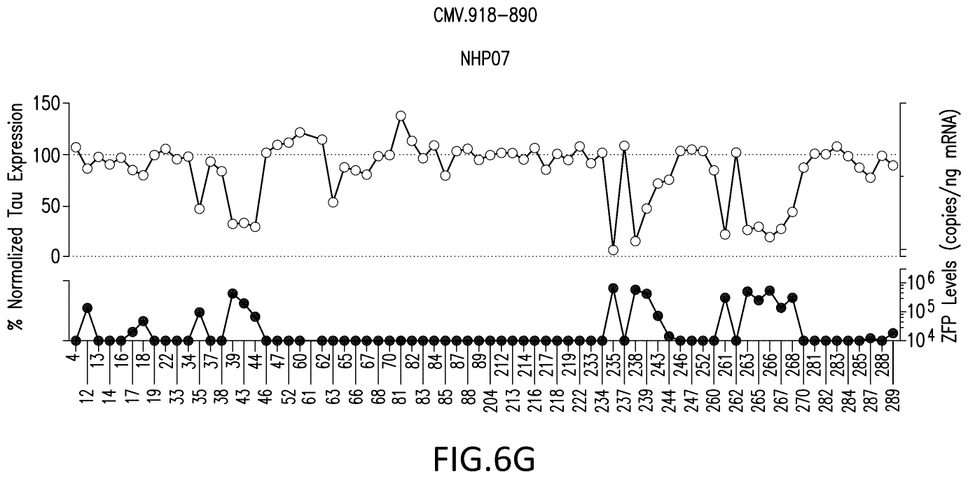

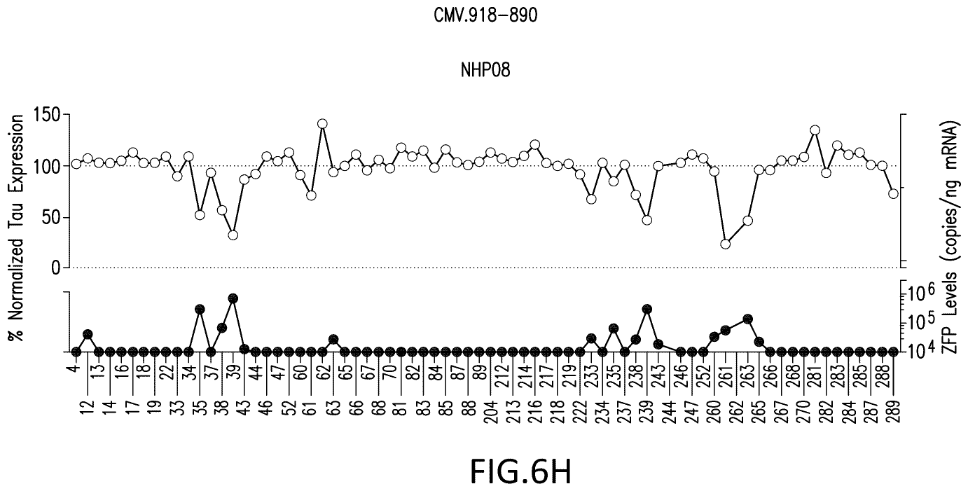

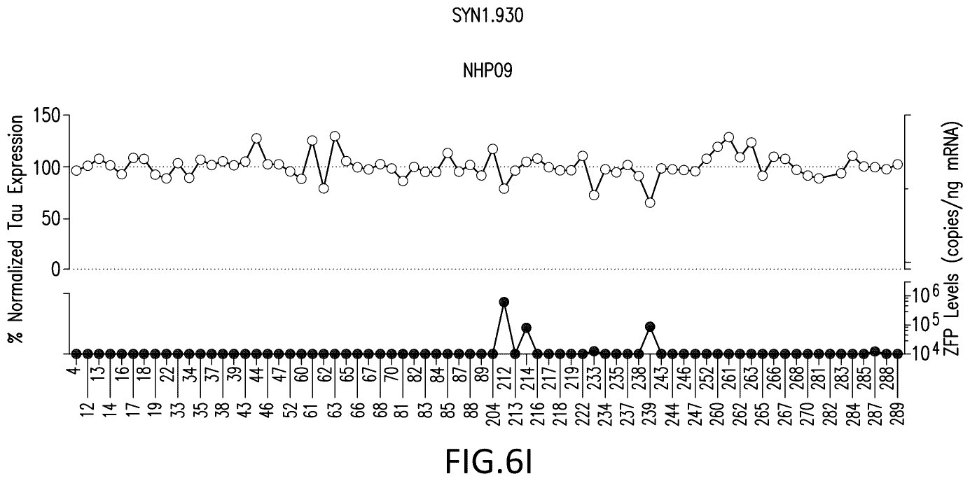

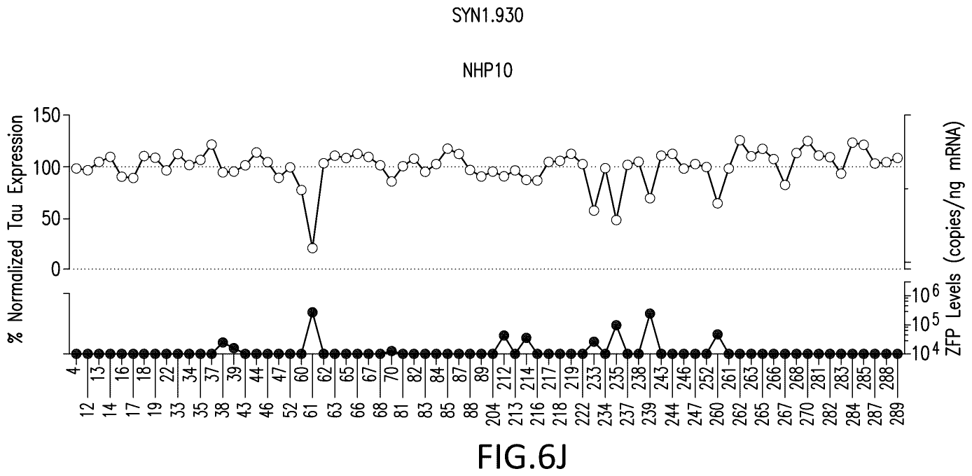

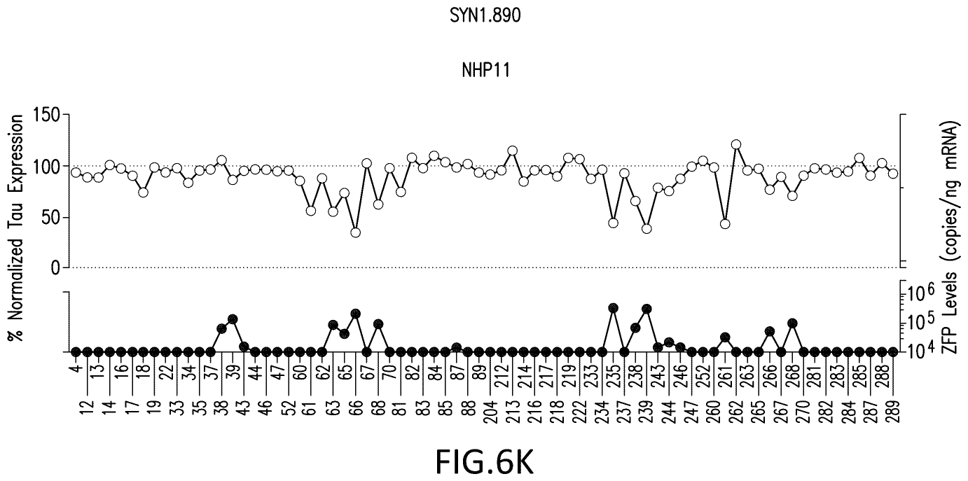

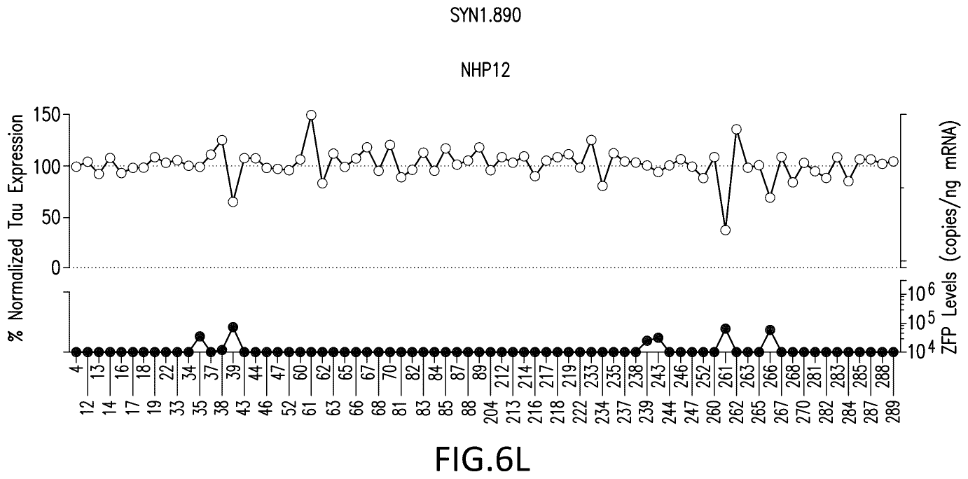

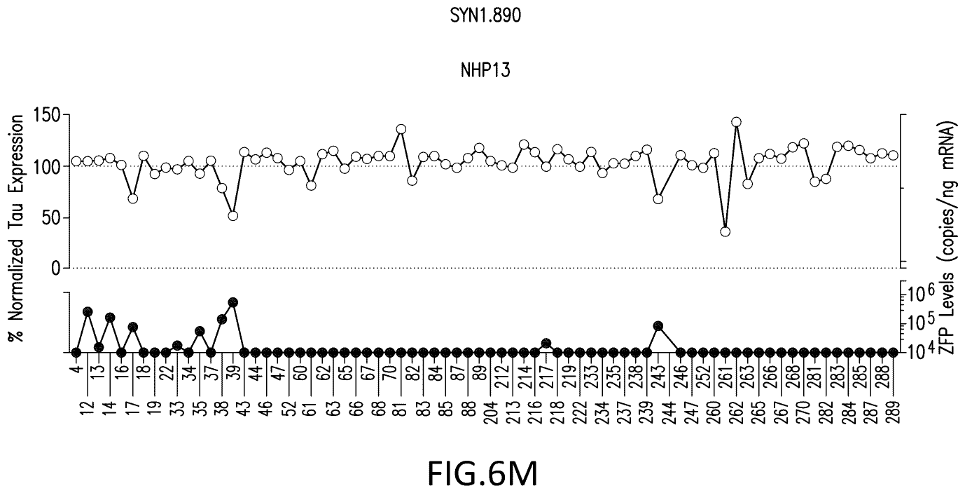

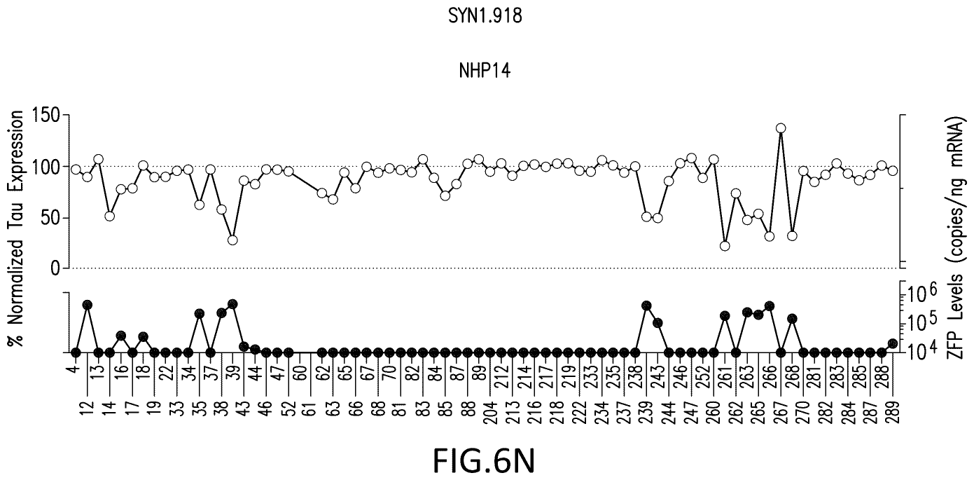

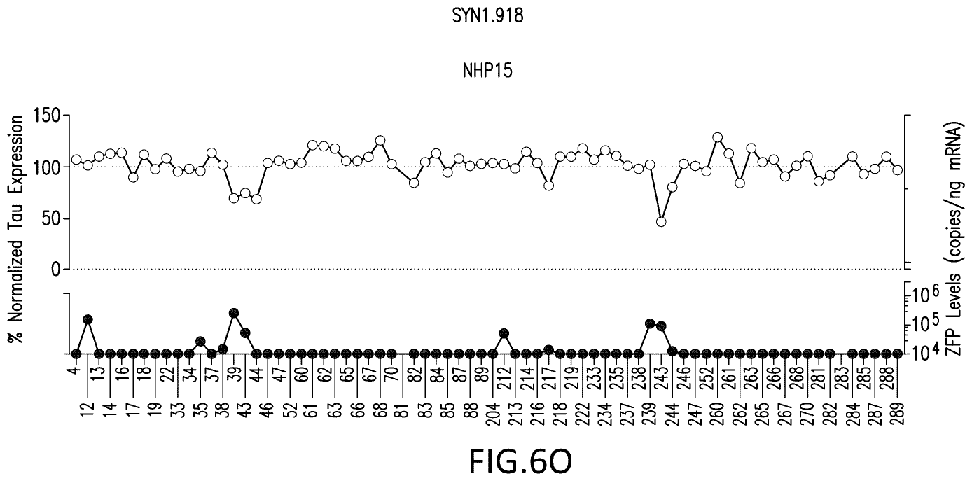

FIG. 6A through FIG. 6O show results of tau expression and ZFP levels in control subjects (FIG. 6A through FIG. 6C; NHP01, NHP02, NHP03); subjects treated with AAV SYN1.918-890 (FIG. 6D through FIG. 6F; NHP04, NHP05, NHP06); subjects treated with AAV CMV.918-890 (FIG. 6G though FIG. 6J; NHP07, NHP08); subjects treated with AAV SYN1.930 (FIG. 6G through FIG. 6J; NHP09, NHP10); subjects treated with AAV SYN1.890 (FIG. 6K through FIG. 6M; NHP11, NHP12, NHP13); and subjects treated with SYN1.918 (FIG. 6N and FIG. 6O; NHP14; NHP15).

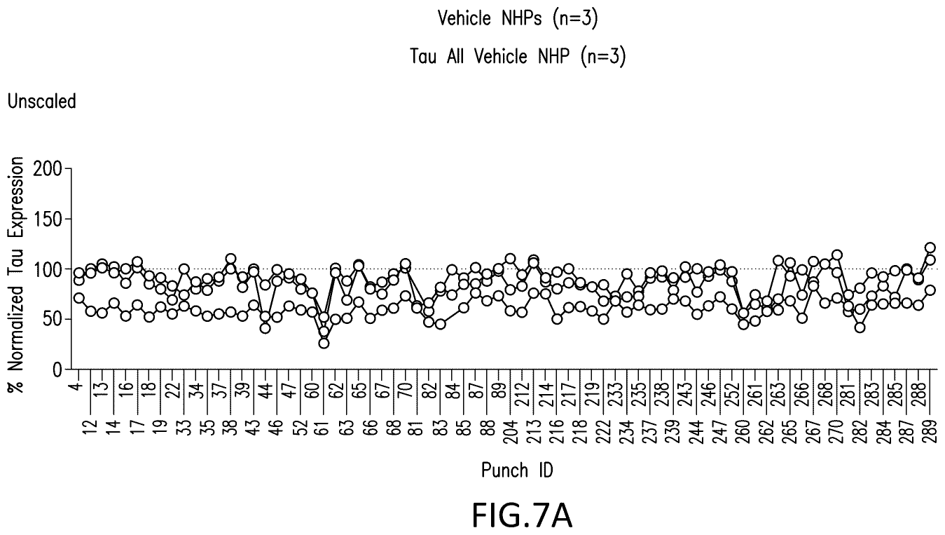

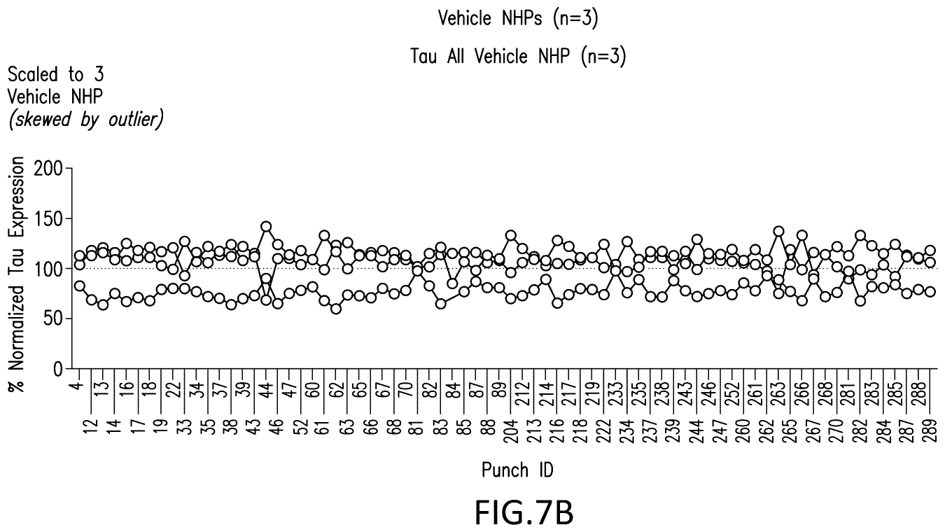

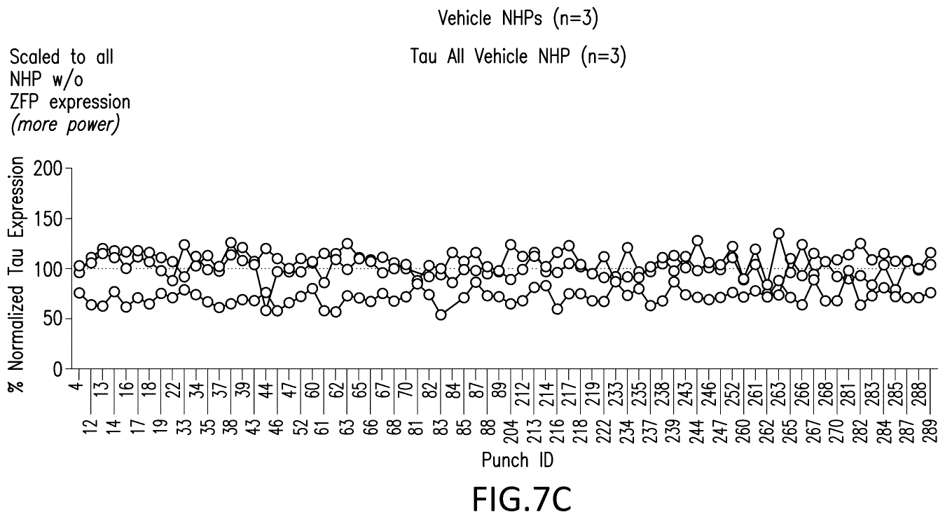

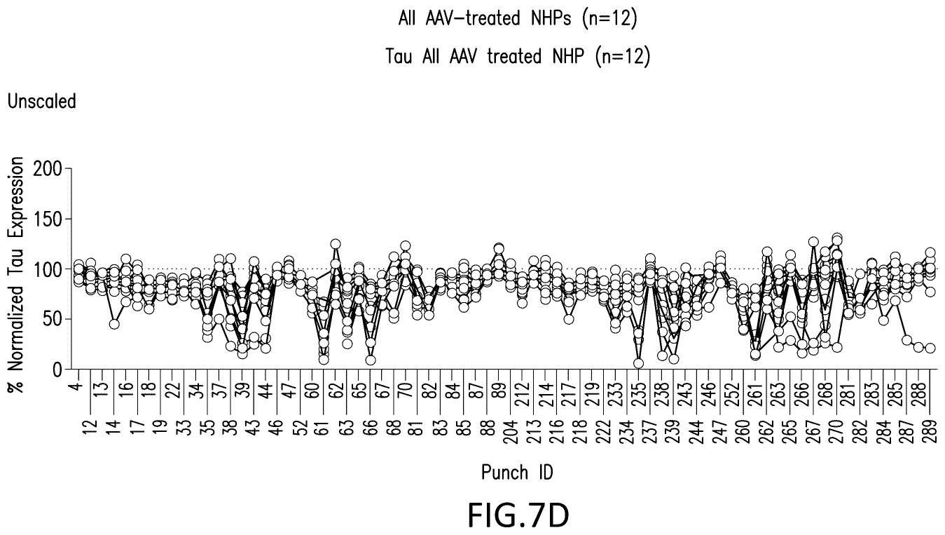

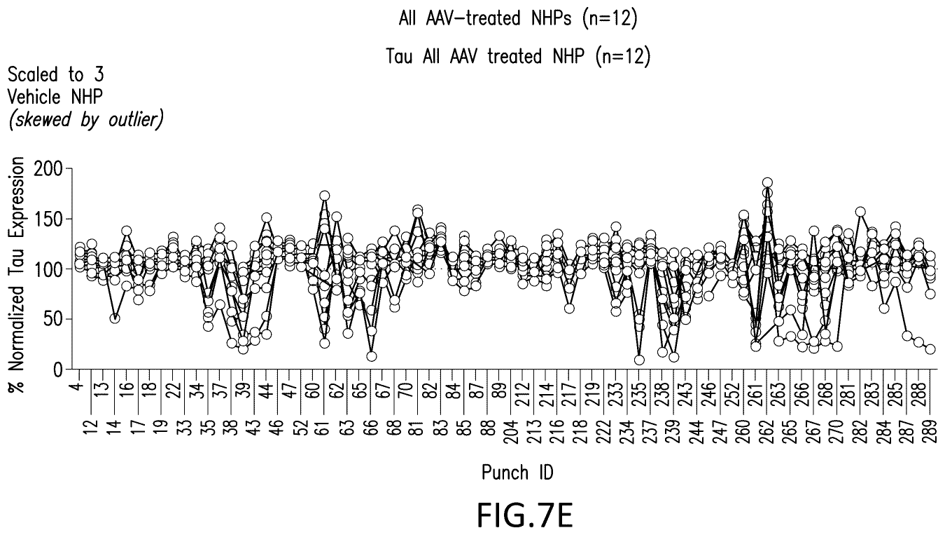

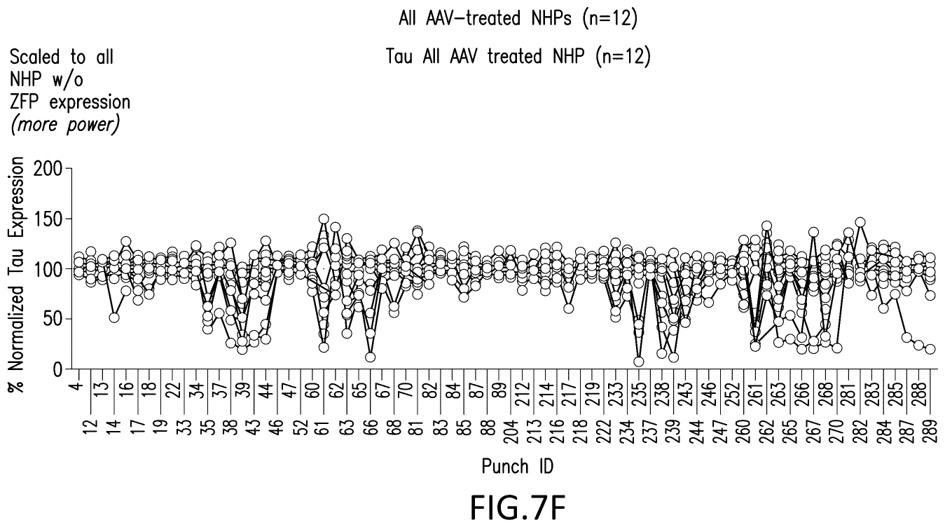

FIG. 7A through FIG. 7F show composite analysis for all punches across all animals evaluating three methods to establish baseline tau levels for a given punch, including no scaling (FIG. 7A and FIG. 7D), scaling to the average of the three vehicle-treated animals (FIG. 7B and FIG. 7E), or scaling to the average tau expression of the vehicle-treated animals and ZFP-treated animals without ZFP expression (FIG. 7C and FIG. 7F). Shown are results from the indicated subjects (vehicle NHP subjects shown in FIG. 7A through FIG. 7C) and all AAV treated subjects (FIG. 7D through FIG. 7F. As shown, scaling by either method better approximates baseline levels across all 15 animals, with the third method (scaling to vehicle- and ZFP-treated NHPs without ZFP expression) being a somewhat better representative of the tau baseline across the 3 vehicle- and 12 ZFP-treated animals in the study.

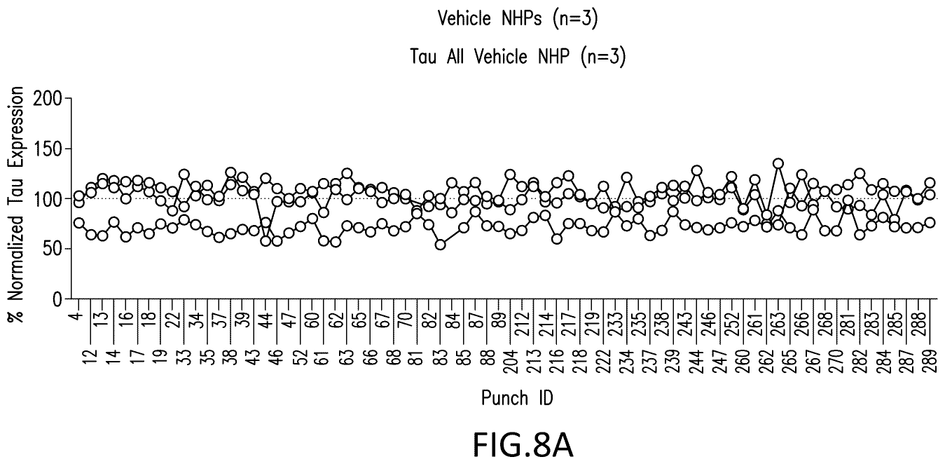

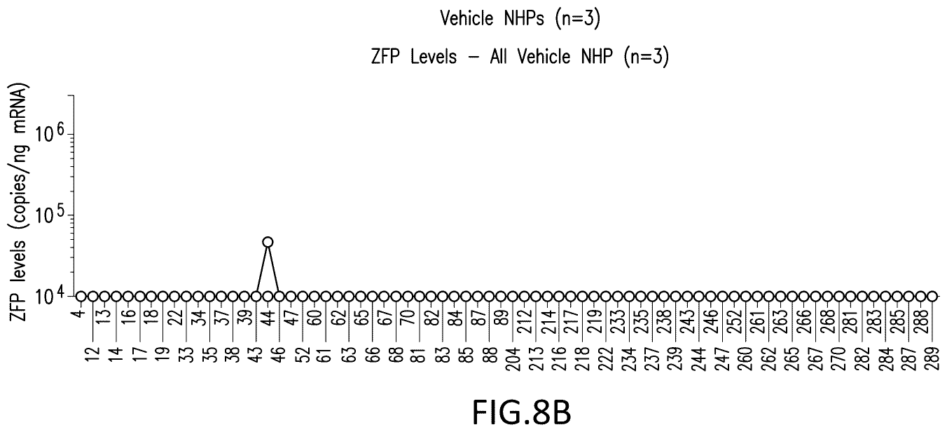

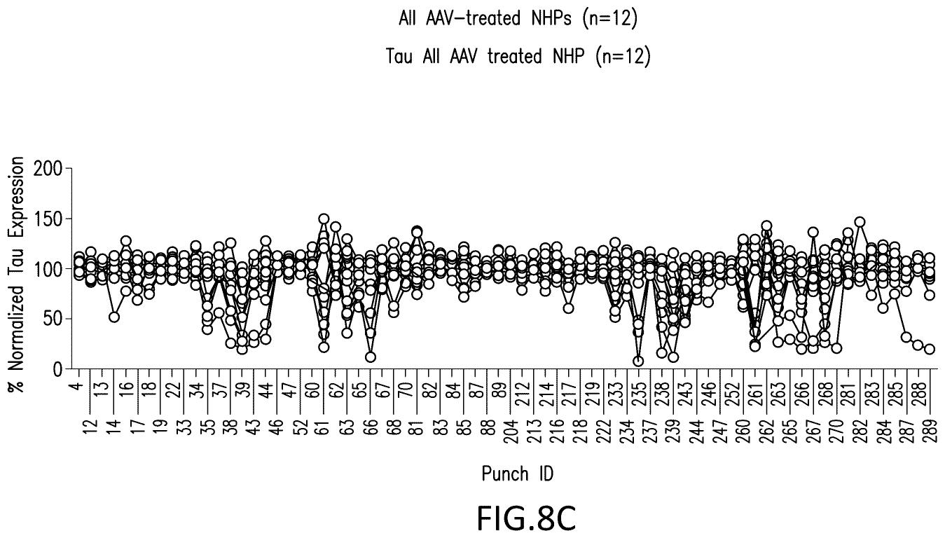

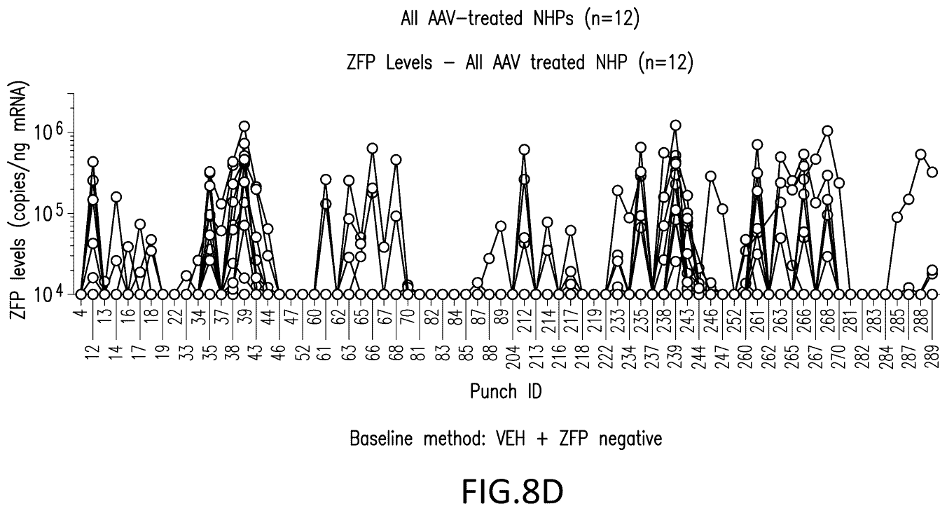

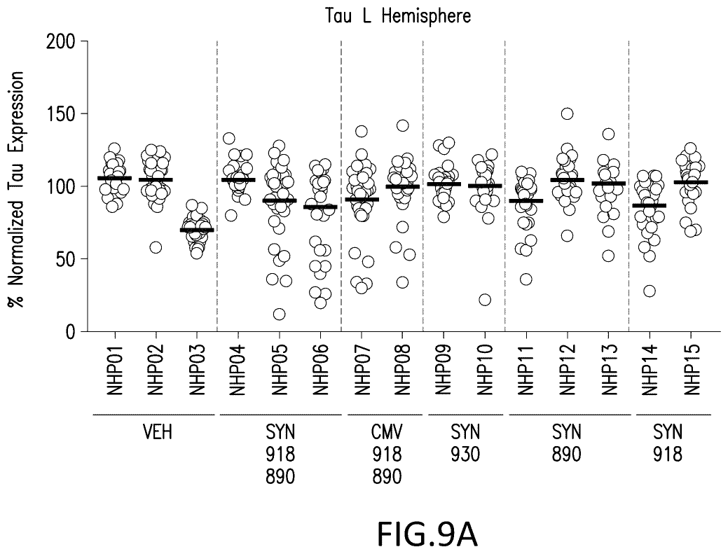

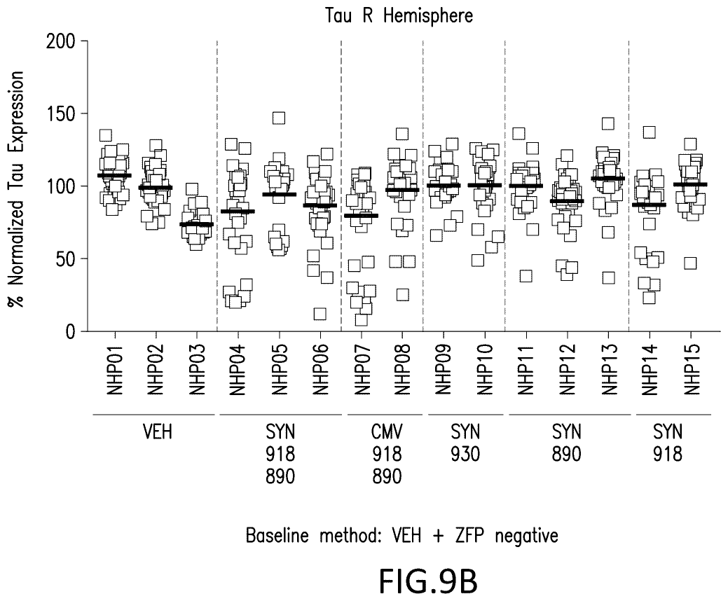

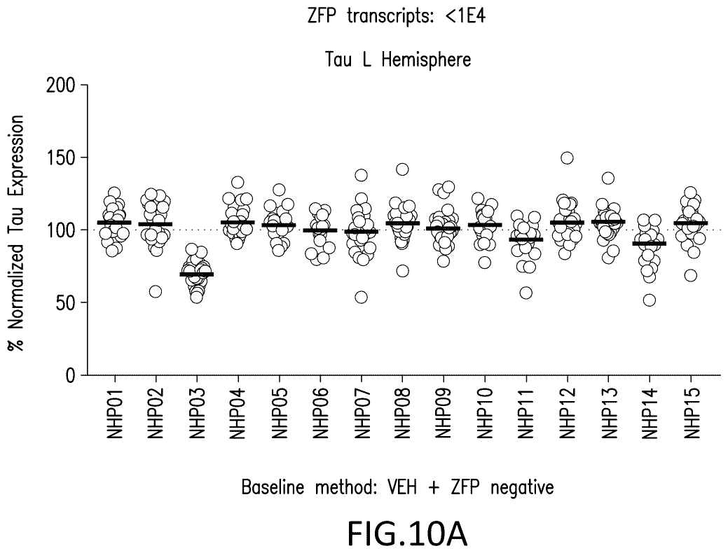

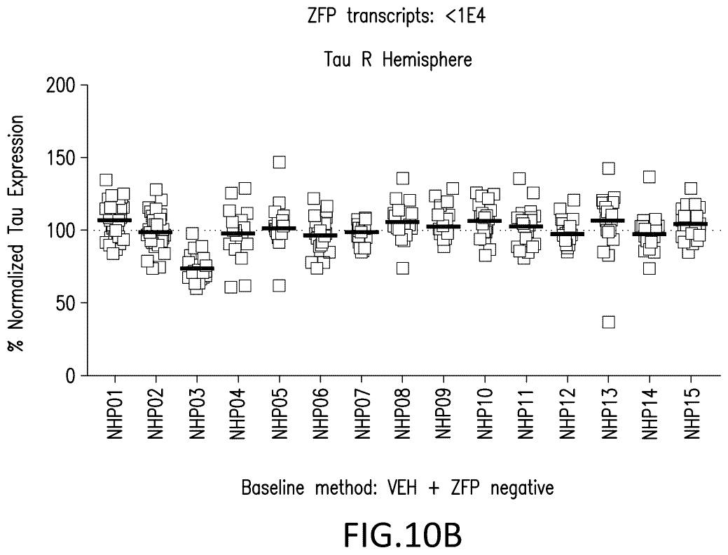

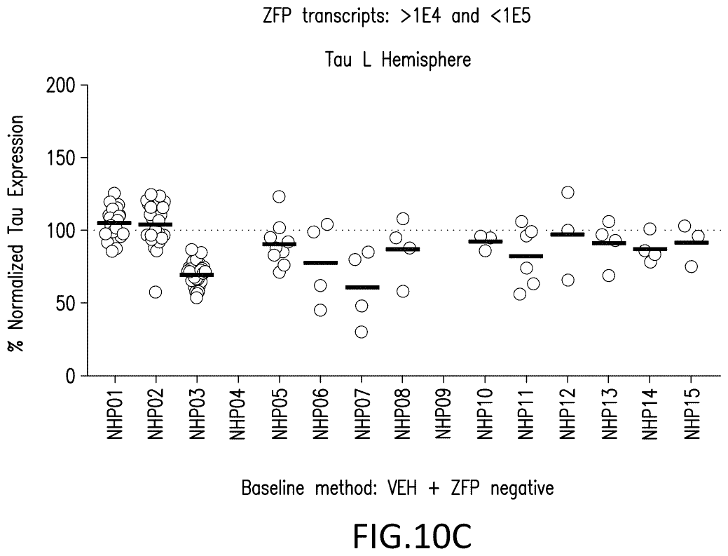

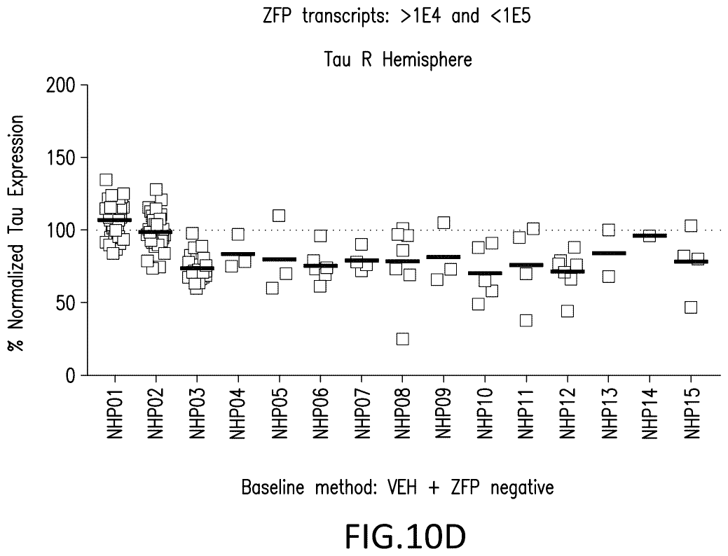

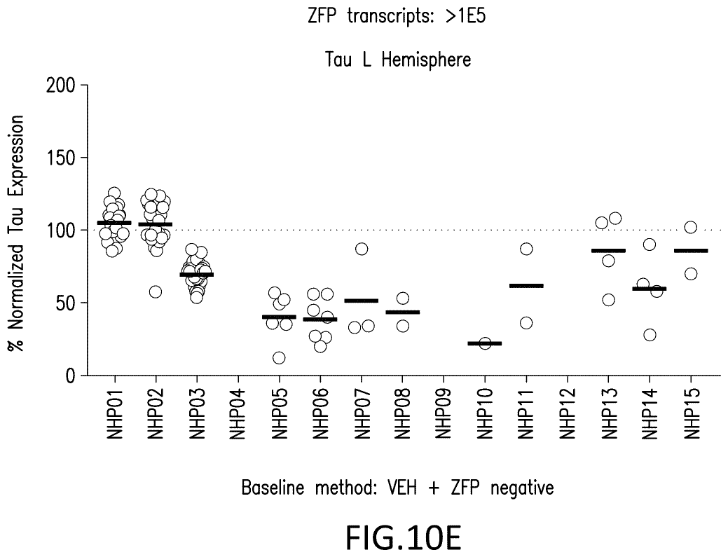

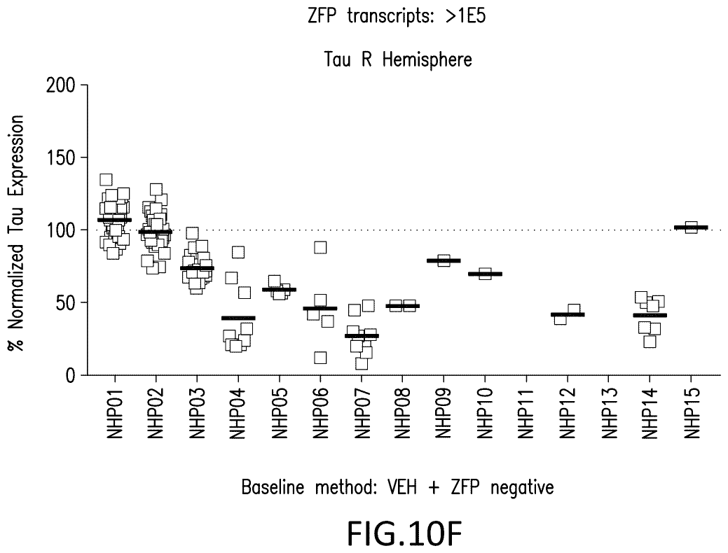

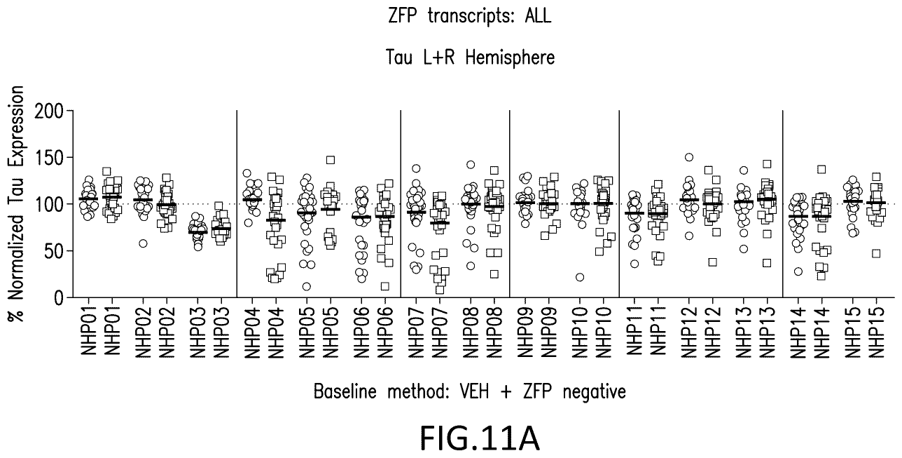

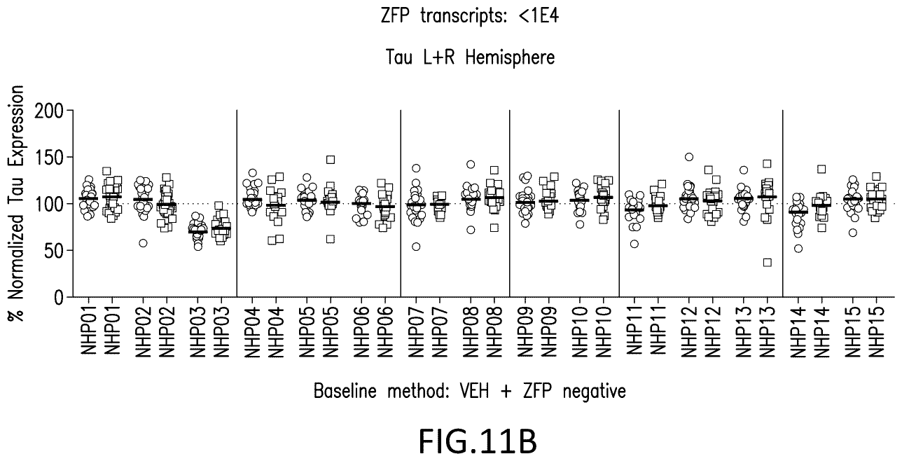

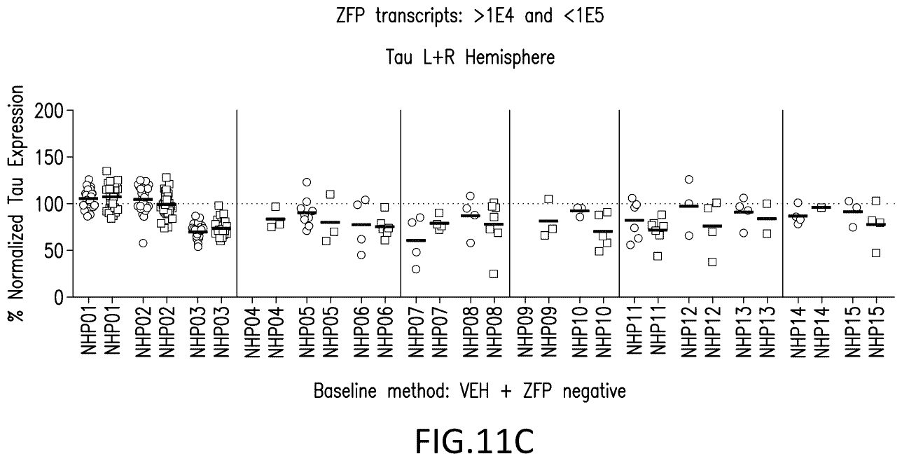

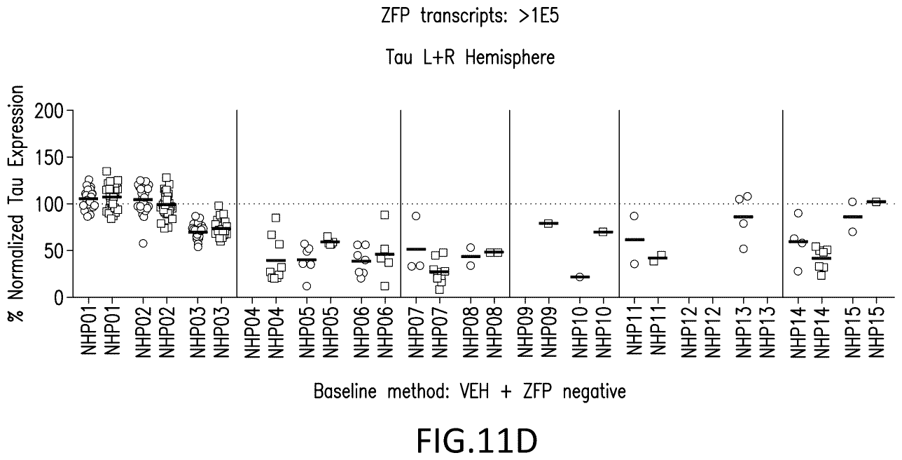

For this analysis, normalized tau expression was scaled to the average of the tau levels measured from vehicle-treated animals and ZFP-treated animals without detectable ZFP expression for each punch. FIG. 8A through FIG. 8D show results from the indicated subjects (vehicle NHP subjects shown in FIG. 8A and FIG. 8B) and all AAV treated subjects (FIG. 8C and FIG. 8D), for both scaled tau expression levels (FIG. 8A and FIG. 8C) and absolute ZFP transcript levels (copies/ng mRNA). FIG. 9A and FIG. 9B show tau expression results from the left and right hemispheres of the indicated subjects. FIG. 10A through FIG. 10F and FIG. 11A through FIG. 11D show results of the indicated animals where less than 1E4 ZFP transcripts were present (FIG. 10A and FIG. 10B, FIG. 11B); between 1E4 and 1E5 ZFP transcripts were present (FIG. 10C and FIG. 10D; FIG. 11C); where greater than 1E5 ZFP transcripts were present (FIG. 10E and FIG. 10F; FIG. 11D) and for all levels of ZFP transcripts (FIG. 11A). As shown, the large majority of punches with low levels of ZFP expression do not have significant tau reduction. However, punches between 1E4 and 1E5 ZFP transcripts/ng mRNA have intermediate tau reduction for some treatments, and those with 1E5 or greater transcripts/ng mRNA have even greater tau reduction.

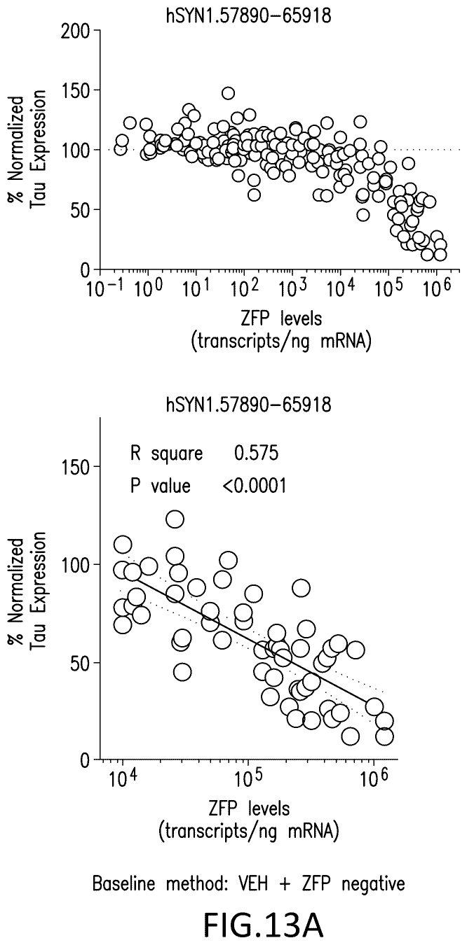

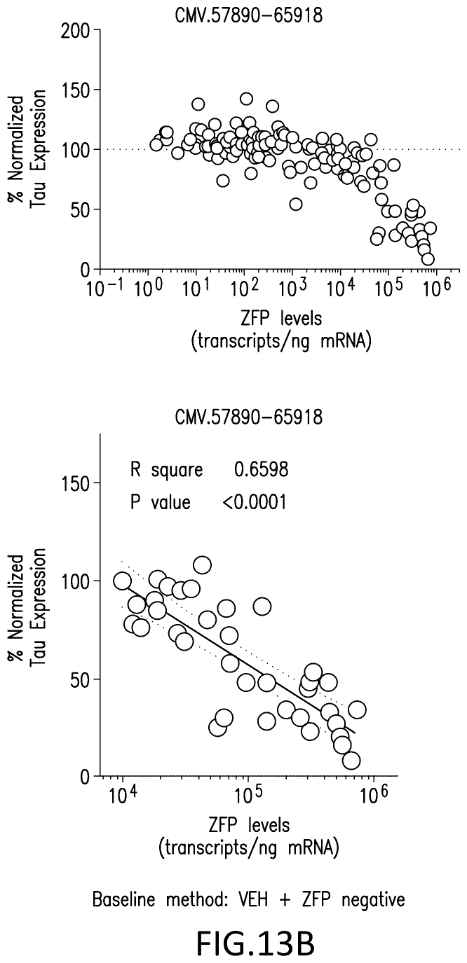

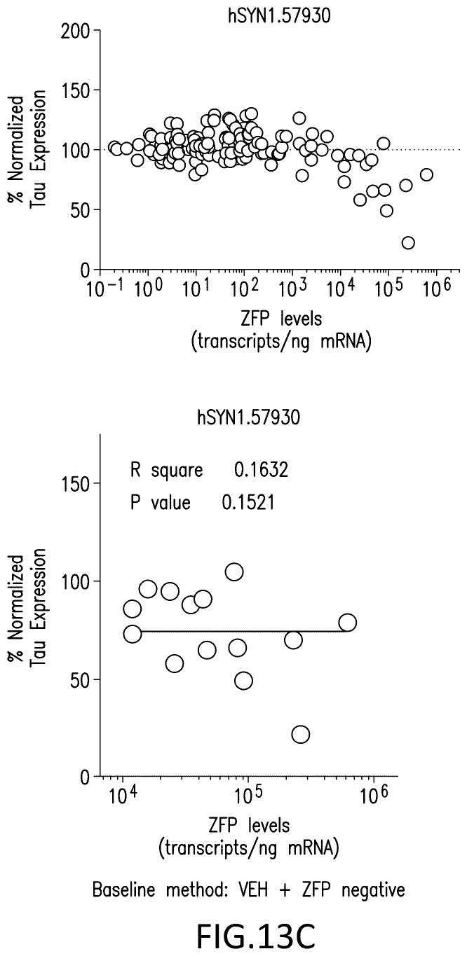

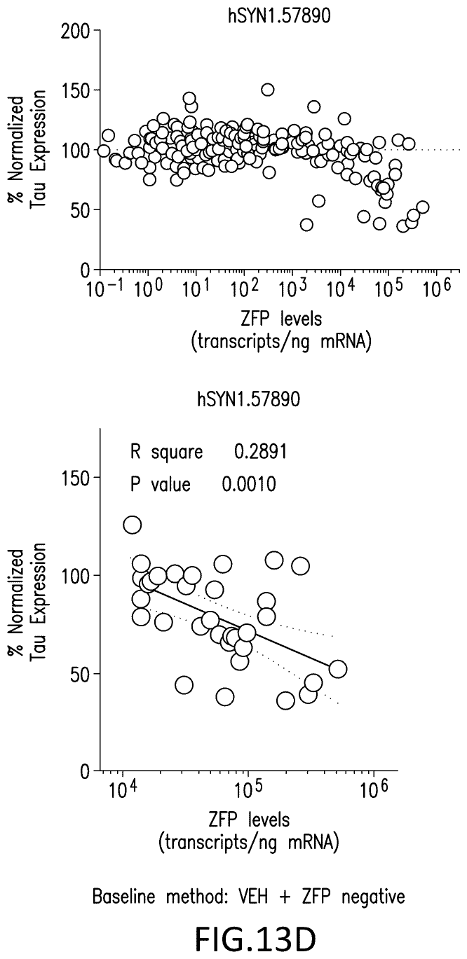

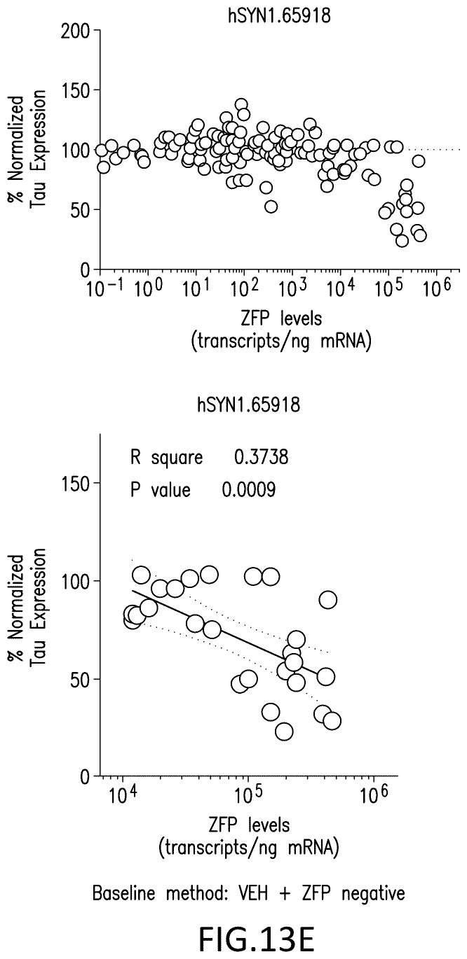

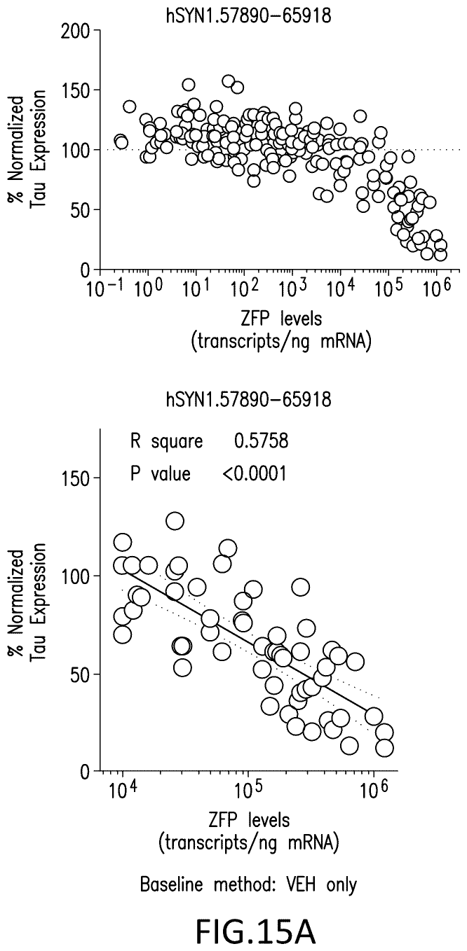

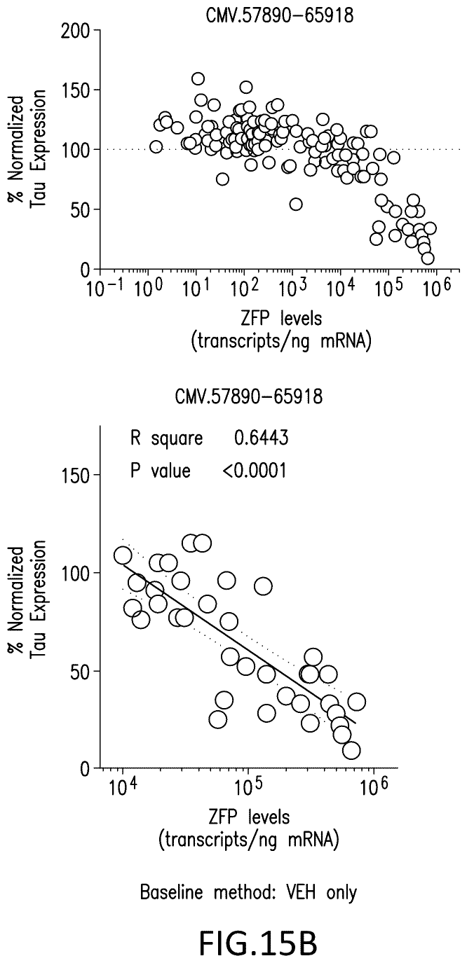

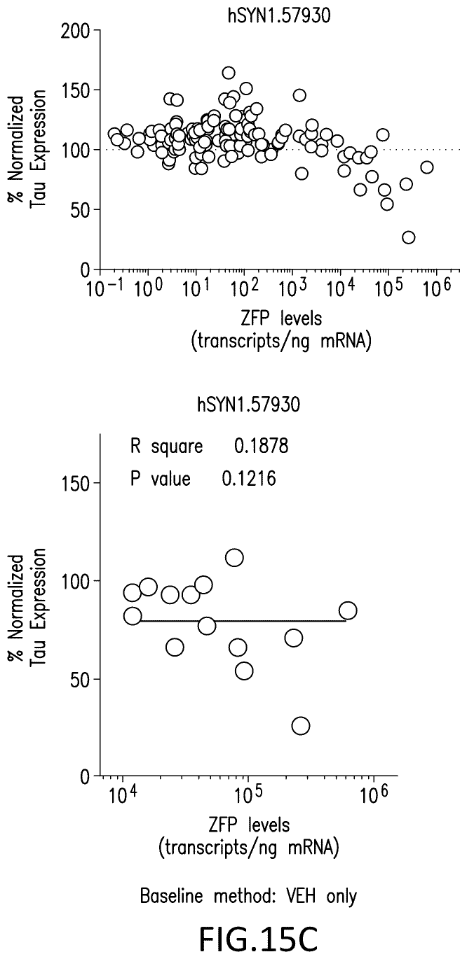

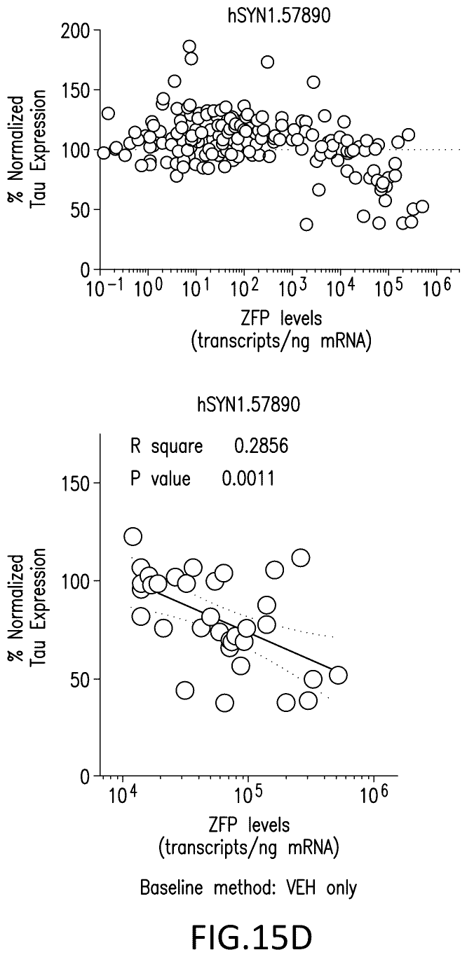

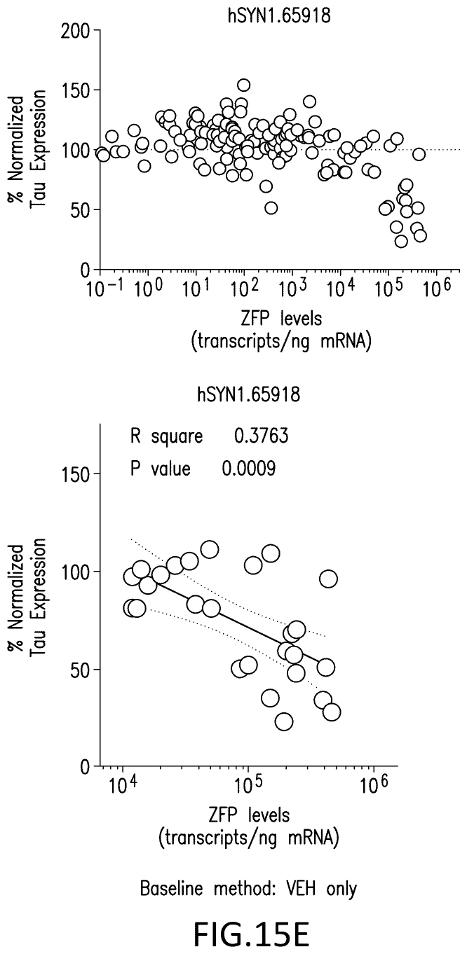

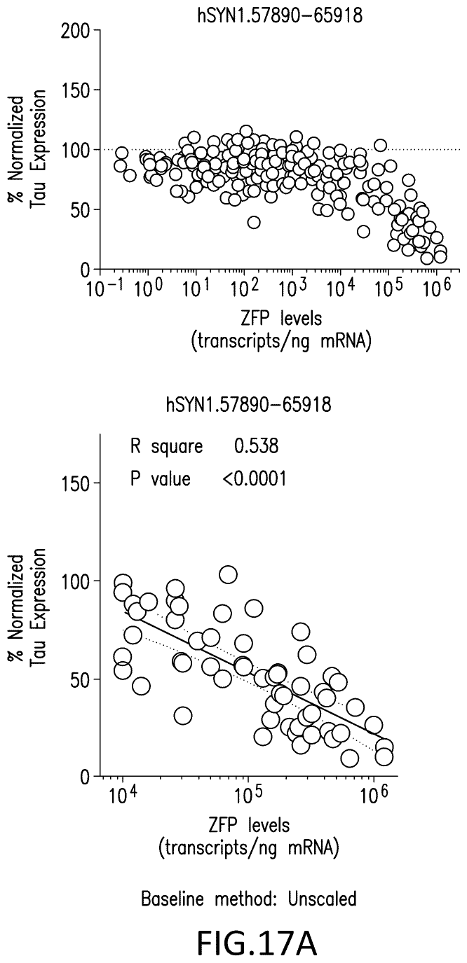

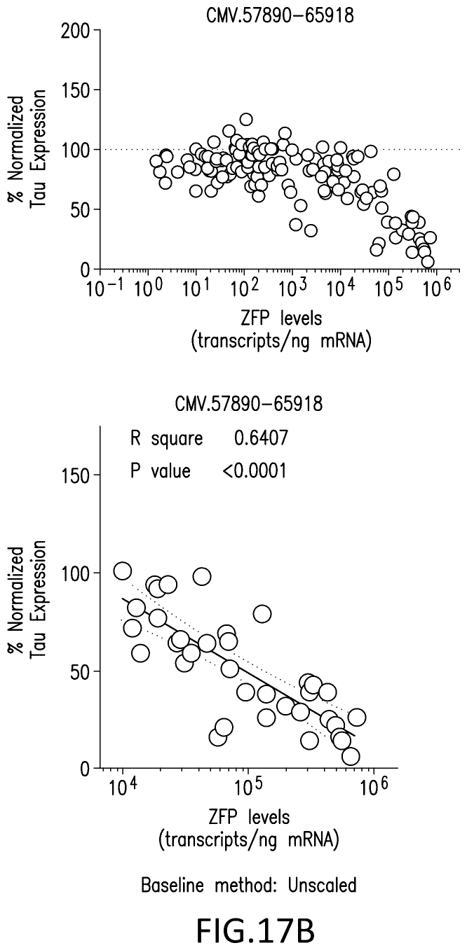

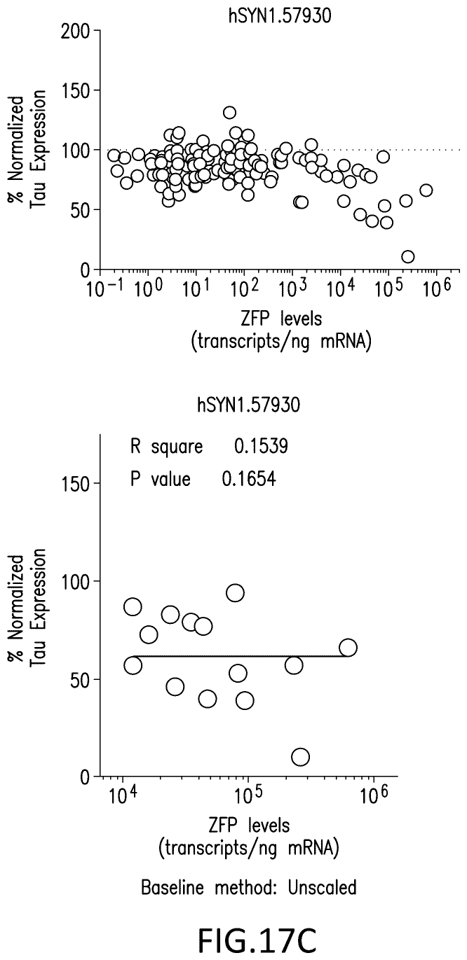

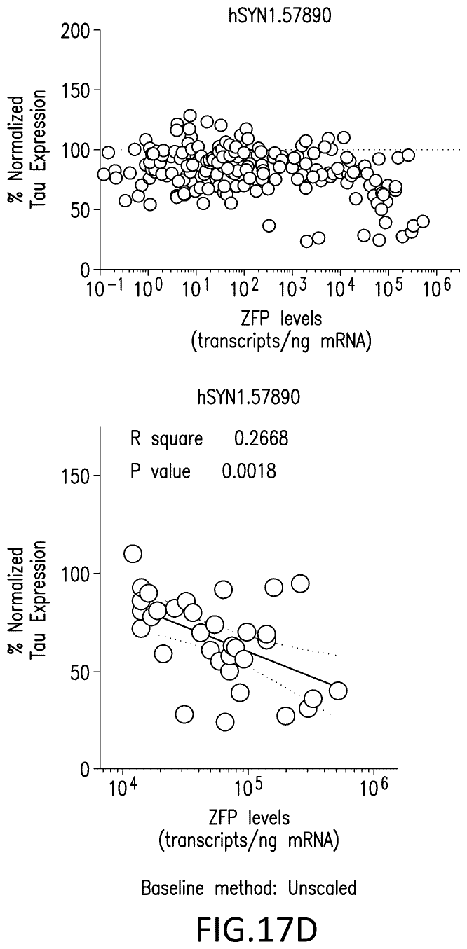

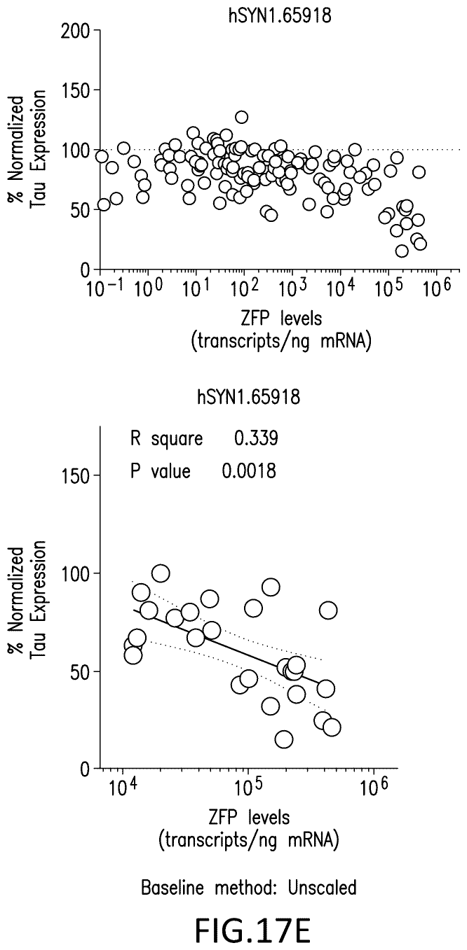

FIG. 13A through FIG. 13E, FIG. 15A through FIG. 15E and FIG. 17A through FIG. 17E show the percent of normalized tau expression as a function of ZFP levels (transcripts/ng mRNA) in subjects treated as indicated. FIG. 13A through FIG. 13E show results where tau expression is scaled to the average of the Vehicle-treated animals and ZFP-treated animals without detectable ZFP expression; FIG. 15A through FIG. 15E show results where tau expression is scaled to the average of the vehicle-treated animals only; and FIG. 17A through FIG. 17E show results where tau expression was not scaled to adjust to a baseline tau level. Regardless of scaling method, the data indicate that there is a significant correlation between ZFP expression level and tau reduction for 4 of the 5 ZFP treatments, namely AAV SYN1.65918-57890, AAV CMV. 65918-57890, AAV SYN1.57890, AAV SYN1.65918. The greatest degree of tau reduction was achieved with the AAV SYN1.65918-57890 and AAV CMV.65918-57890 treatments, with some punches exceeding >80% tau reduction.

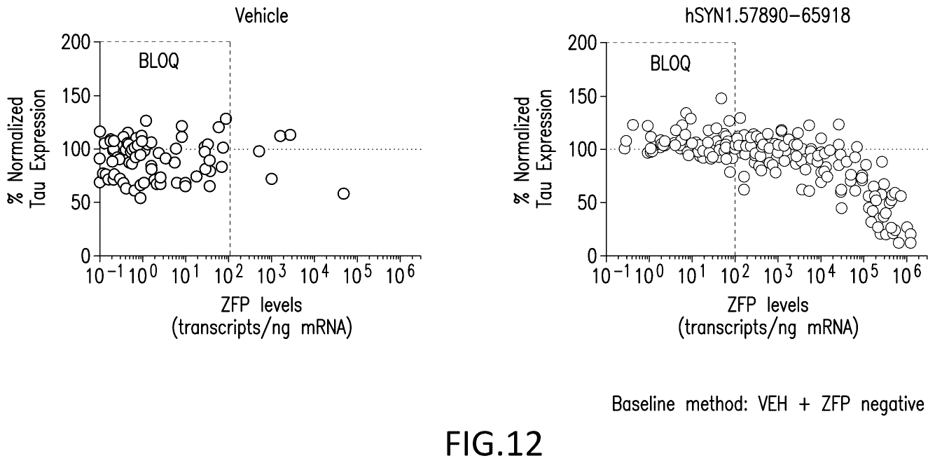

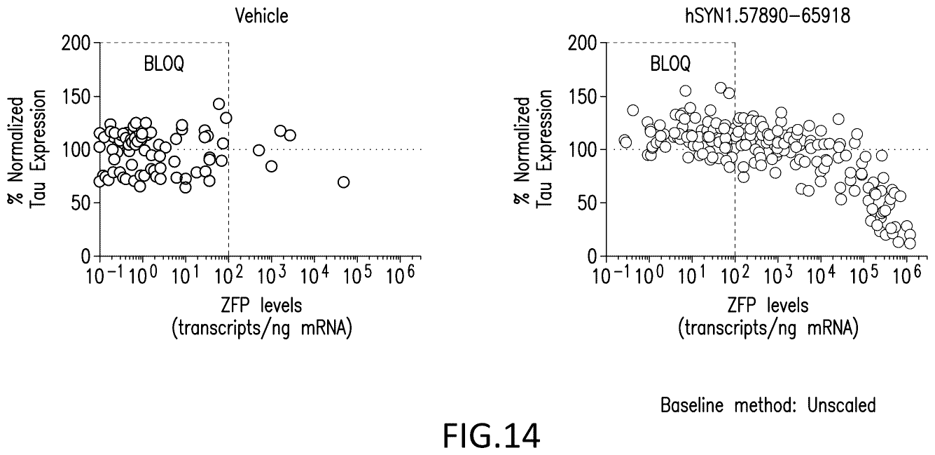

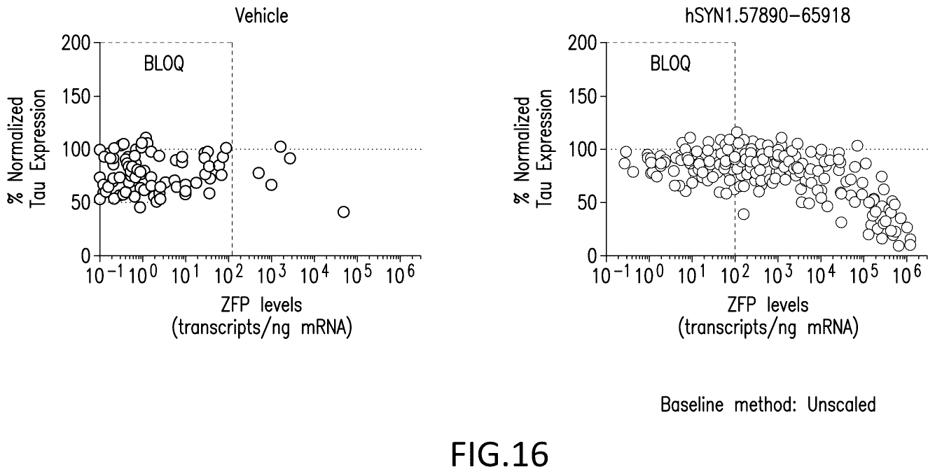

FIG. 12, FIG. 14 and FIG. 16 show tau expression vs. ZFP level correlation plots for NHP subjects treated with either vehicle (left panel) or AAV vectors encoding 57890 and 65918 ZFP-TF repressors whose expression is driven by the synapsin 1 promoter (right panel). The limit of the ZFP qRT-PCR assay for absolute quantitation is approximately 1E2 transcripts/ng mRNA which is indicated by Below Limit of Quantitation (BLOQ). FIG. 12 shows results where tau expression is scaled to the average of the vehicle-treated animals and ZFP-treated animals without detectable ZFP expression. FIG. 14 shows an analysis where tau expression levels are scaled to the average of the three vehicle-treated animals. FIG. 16 shows an analysis where tau expression levels were not scaled to correct for tau baseline levels. As shown repression of tau was correlated to amount of ZFP-TF present.

### Example 2: Tau Repression in Humanized Tau Mouse Model

Tau reduction in the P301L mutant human tau (P301L) transgenic mouse model of tauopathy (rTg4510, Jackson Labs) and the hTau mouse model (B6.Cg-Mapt^{tm1(EGFP)Klt}Tg(MAPT)8cPdav/J, Jackson Labs) was also assessed following administration of genetic repressors as described herein. hTau mice express WT human MAPT gene and, further, the endogenous (mouse) Mapt gene knocked out and replaced with a GFP expressing construct. Treatment groups were as shown in the following Table:

| | | | No. of | Time |
|---|---|---|---|---|
| Treatment | RoA* | Dose | mice | Point |
| Vehicle (PDS) | Ipa** (hippocampus) | 0 | 6 | 6 wk |
| AAV9.hSYN1.57890 | IPa (hippocampus) | 3E10 | 5 | 6 wk |
| AAV9.hSYN1.65918- | IPa (hippocampus) | 3E10 | 6 | 6 wk |
| T2a-57890 | | | | |

*Route of Administration
**Intraparenchymal (Ipa)

Endpoints measured were as follows: ZFP and Tau mRNA expression levels by RT-qPCR; GFAP, Iba1, NeuN mRNA expression levels by RT-qPCR; Saitohin (STH) mRNA expression levels by RT-qPCR (STH is a protein-encoding gene in apes and humans that is nested in the intron between exons 9 and 10 of the human tau gene, see, e.g., Conrad et al. (2002) Proc Natl Acad Sci USA. 99(11):7751-7756.); and tau protein levels.

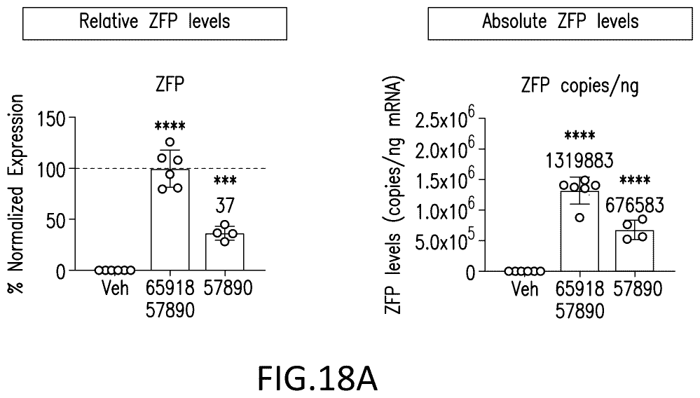

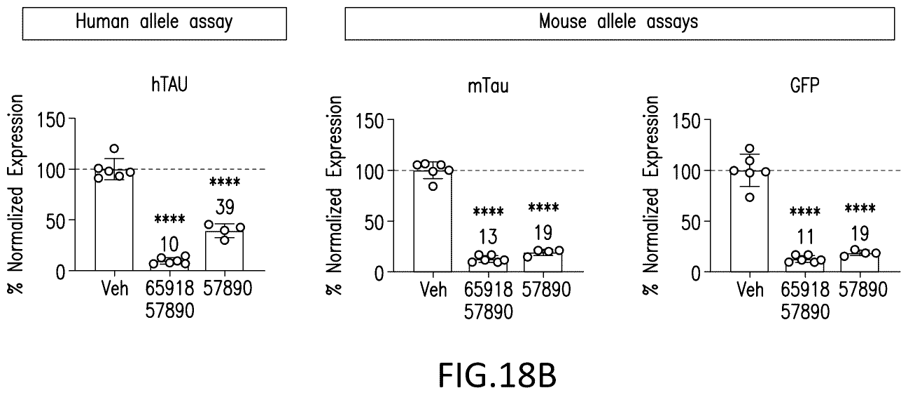

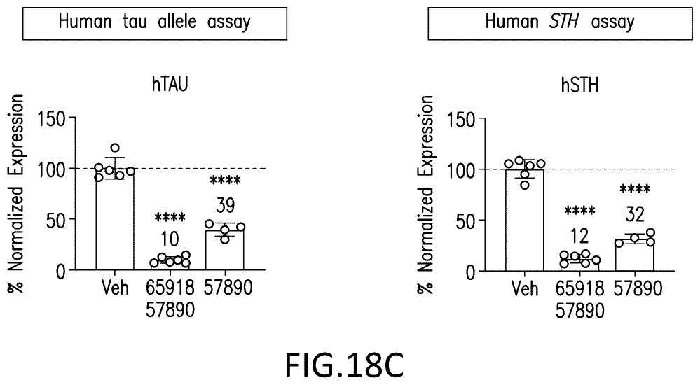

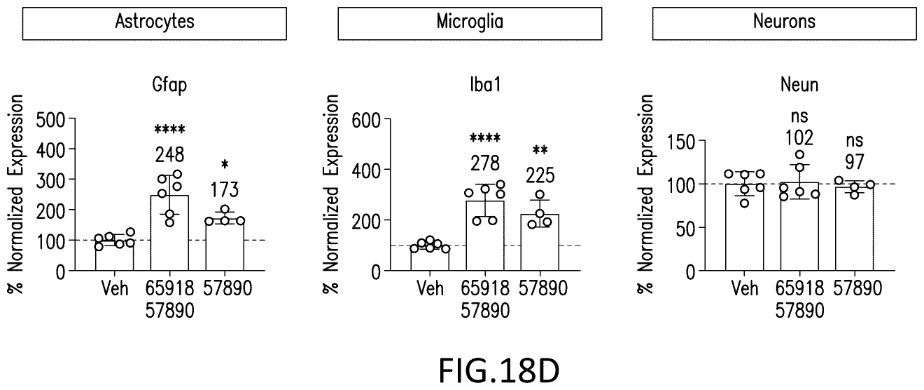

As shown in FIG. 18A through FIG. 18D, the synergistic pairing of 57890 and 65918 (AAV9.hSYN.65918-T2a-57890) resulted in ZFP expression levels that were at least 2-fold higher than 57890 (AAV9.hSY1.57890) alone. See, FIG. 18A. In addition, in humanized tau mice administered the construct encoding 57890 and 65918, human tau mRNA was repressed by approximately 90% repression as compared to ˜60% repression in mice administered the construct encoding only 57890 (mouse tau mRNA expression was repressed by ˜87% by 57890 and 65918 combination and ˜81% by 57890). See, FIG. 18B. Similar results were obtained following RT-qPCR analysis of human Saitohin (hSTH), with repression by the synergistic combination of 57890 and 65918 at ˜88% as compared to ˜68% using 57890 alone. See, FIG. 18C. IBA1 and GFAP levels were elevated in both treatment groups as compared to control but NeuN levels were not significantly different between groups. See, FIG. 18D.

In addition, the clinical and therapeutic effectiveness of the repressors is further evaluated in this and other mouse models of AD (e.g., APPswe/PS1d9, Jackson Labs) to determine whether there is a reduction in biomarkers and symptoms of tauopathies, including by one or more the following: RNAscope ISH analysis (single cell analysis of ZFP, human tau, and mouse tau, see, e.g., Carstens et al. (2016) J Neurosci. 36(23):6312-6320); IHC ZFP/tau analysis (see, e.g., Zeitler et al. (2019) Nature Medicine 25:1131-1142); neurotoxicity, gliosis, dystrophic neurites, spine loss, excitotoxicity, cortical and hippocampal shrinkage, dendritic tau accumulation, cognitive (e.g., the radial arm maze and the Morris water maze, fear conditioning, etc.), and motor deficits. See, e.g., Bryan et al., (2009) Chapter 1: Transgenic Mouse Models of Alzheimer's Disease: Behavioral Testing and Considerations in Methods of Behavior Analysis in Neuroscience. 2nd edition, ed. Buccafusco, Boca Raton (FL): CRC Press/Taylor & Francis. Additionally, chemically induced seizure models, for example, wild-type mice treated with excitotoxic compounds such as pentylenetetrazole (PTZ, see e.g. Myers et al. (1975) Epilepsia 16(2):257-67) or kainate (Ferraro et al. (1997) Mamm Genome 8:200-208, are also assessed at 4-8 weeks following administration of genetic repressors as described herein, to determine whether tau reduction confers a protective effect against seizure, including, fatality related to seizure, prolonged latency to seizure, and/or reduction in seizure severity.

### Example 3: Neuroinflammatory Responses

Primates treated in vivo with MAPT repressor ZFP-TFs were also evaluated for the expression levels of microglial and astrocyte markers. In particular, punches as described in Example 1 were evaluated using RT-qPCR reagents for IBA1 and GFAP expression. In addition, levels of the E1F4A housekeeping gene was also evaluated in treated primates. Briefly, brain punches were transferred to 1.5-mL Eppendorf tubes containing 0.6 mL TRI reagent (Thermo Fisher) and two 3.2-mm steel beads (BioSpec Products) on ice.

The tissue was lysed using a Qiagen TissueLyser at 4° C. using the following parameters: 5 cycles, 90 s duration, 25.1 frequency. After brief centrifugation, 70 μL of 1-bromo-3-chloropropane was added to each sample at RT. The samples were vortexed for 10 s, centrifuged at 12,000×g for 10 min at 4° C., and 120 μL of the aqueous phase from each sample was transferred to wells of a 96-well plate. Sixty microliters of isopropyl alcohol and 12 μL of MagMax magnetic beads (Thermo Scientific) were added to each sample well containing the aqueous phase samples. A Kingfisher 96 robot (Thermo Scientific) and the MagMax kit (Thermo Fisher) were used to isolate RNA from the tissue lysate following the manufacturer's instructions. One hundred microliters of the eluted RNA were separated from the magnetic beads using a magnetic stand.

RNA yield and quality were evaluated using a Nanodrop 8000 instrument (Thermo Scientific). cDNA was prepared using the High-Capacity cDNA Reverse Transcription Kit (Applied Biosystems), with 10 μL of RNA and 10 μL of RT Master Mix (10x RT buffer, 10× random primer, 25×dNTP mix, Multiscribe enzyme, and RNAse-free water) for all samples. Reverse transcription was performed on a C1000 Touch Biorad thermal cycler using the following program: 25° C. for 10 min, 37° C. for 120 min, 85° C. for 5 min, and hold at 4° C. qRT-PCR was performed using Biorad CFX384 thermal cyclers. cDNA was diluted 10-fold in nuclease-free water, and 4 μL of diluted cDNA were added to each 10 μL PCR reaction. Each sample was assayed in technical quadruplicate. Custom Taqman primer:probe assays were used in this study. 2× Fast Multiplex PCR (Qiagen) master mix was used for the tau/EIF4a2/ATP5b triplex assay, and SsoAdvanced Universal Probes Supermix (Biorad) was used for the other assays. The qPCR cycling conditions were as follows: Qiagen Fast Multiplex master mix →95° C. for 5 min, 95° C. for 45 s, 60° C. for 45 s, plate read, 40 cycles; Biorad SsoAdvanced master mix →95° C. for 90 s, 95° C. for 12 s, 60° C. for 40 s, plate read, 42 cycles.

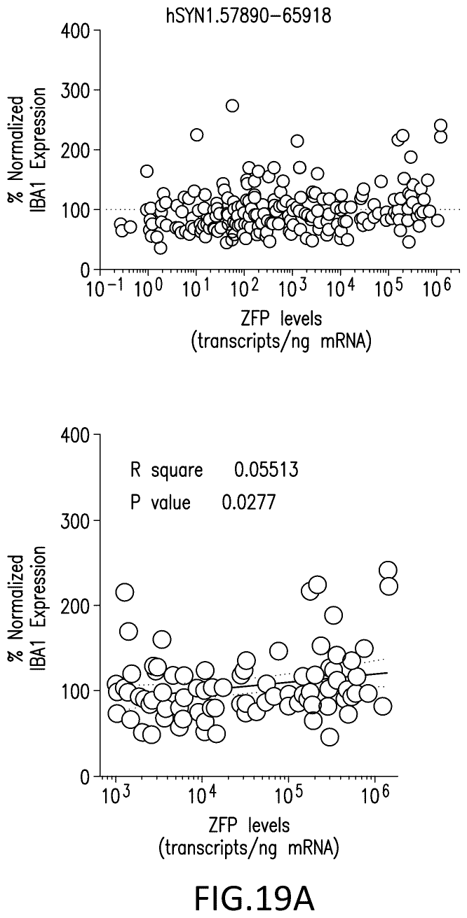

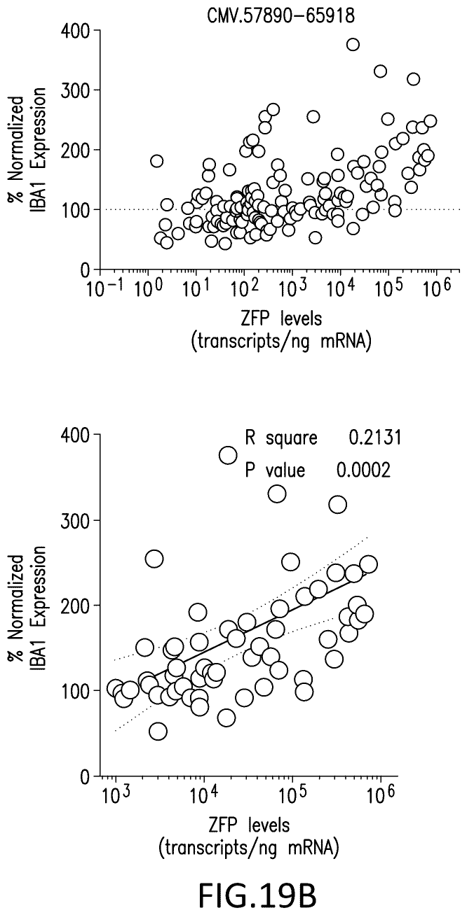

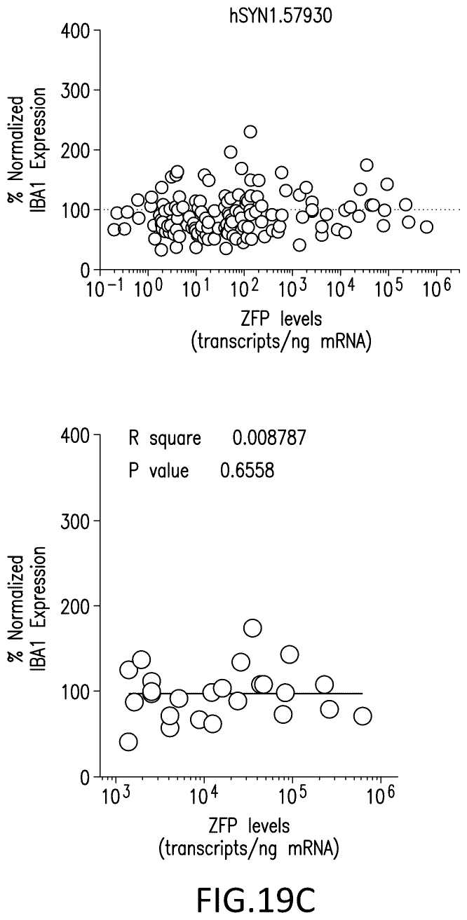

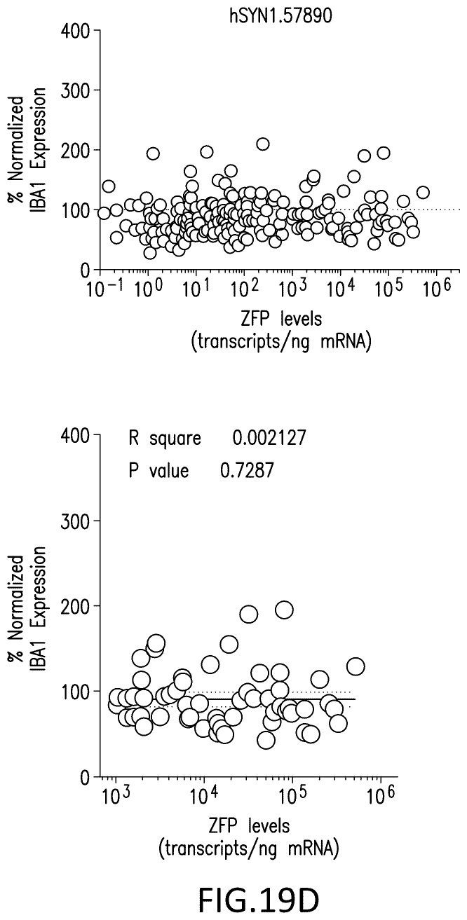

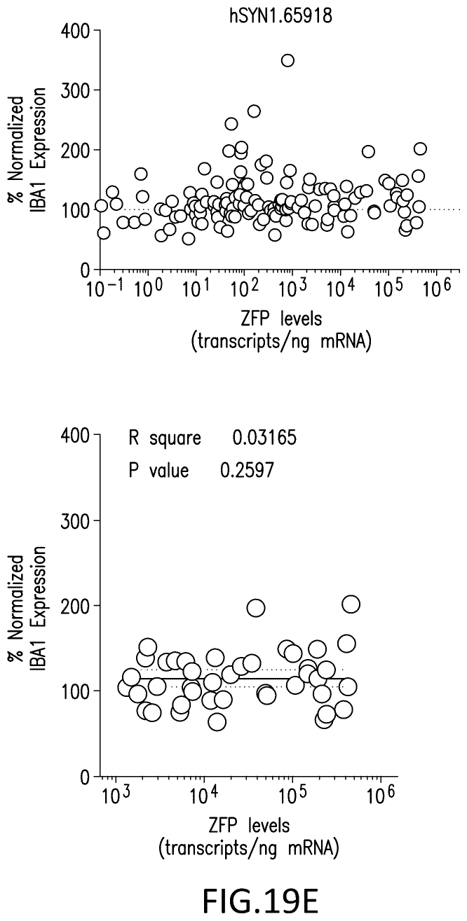

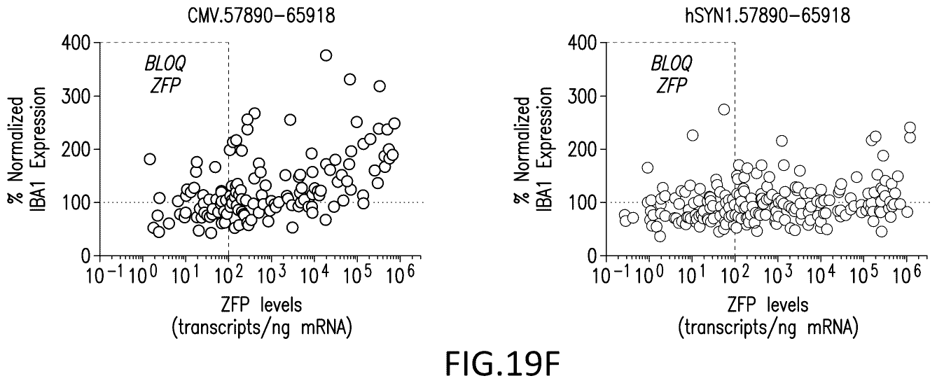

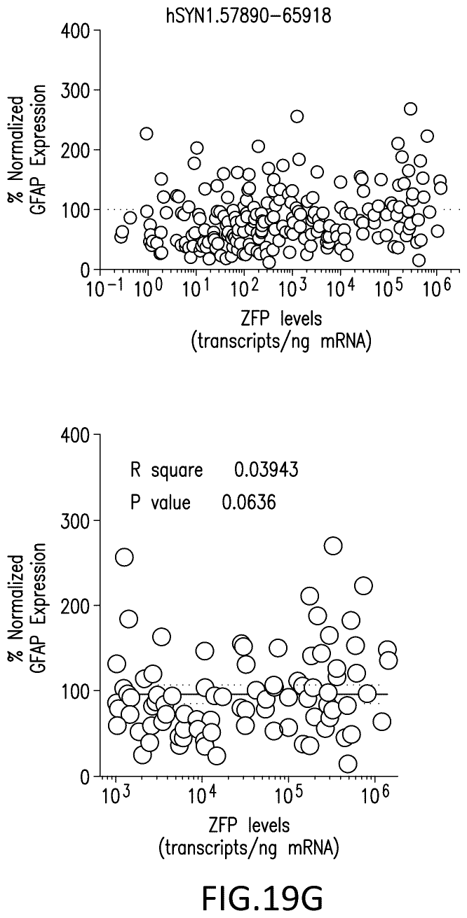

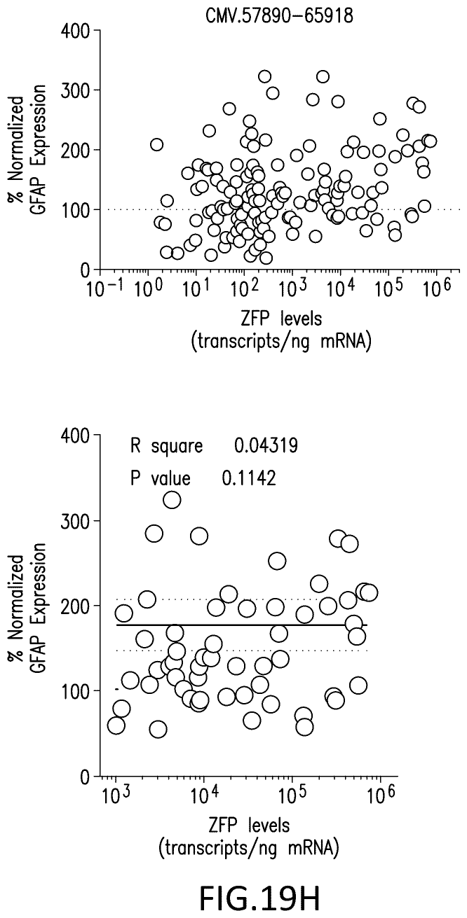

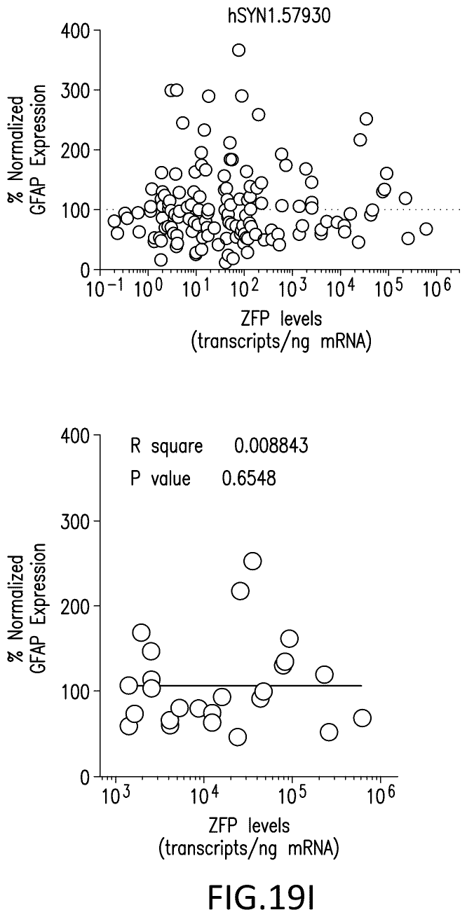

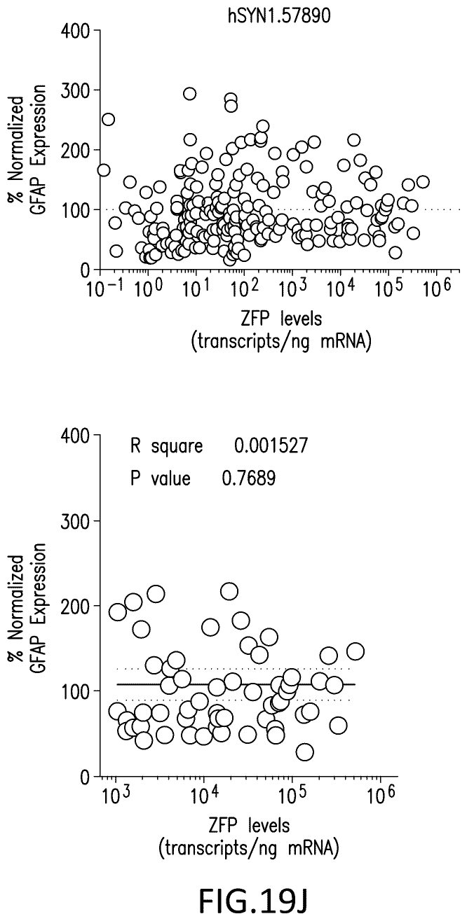

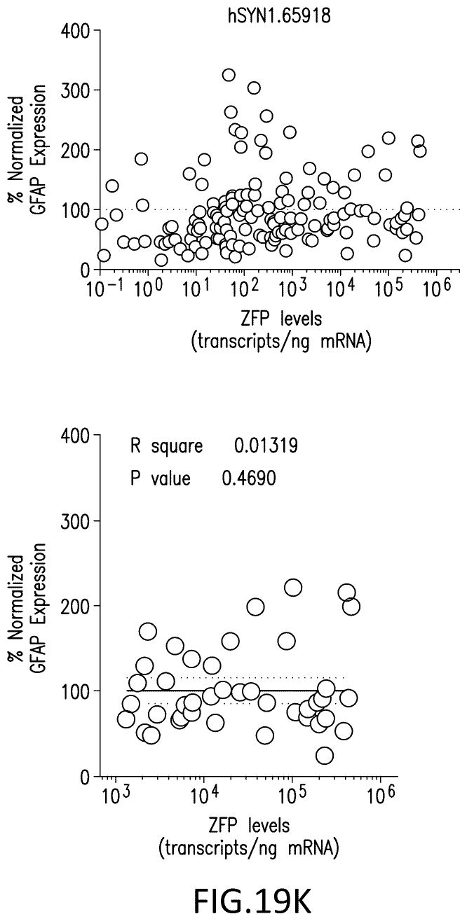

As shown in FIG. 19A through FIG. 19K, IBA1 and GFAP analysis showed no ZFP-dependent elevation in microglial or astrocyte marker levels in any of the primates treated with AAV constructs including the synapsin promoter. However, primates treated with AAV construct including the CMV promoter showed elevated astrocyte marker levels. Thus, no ZFP-dependent neuroinflammatory responses were observed when constructs comprising the neuronal-specific Synapsin promoter were administered to the primates.

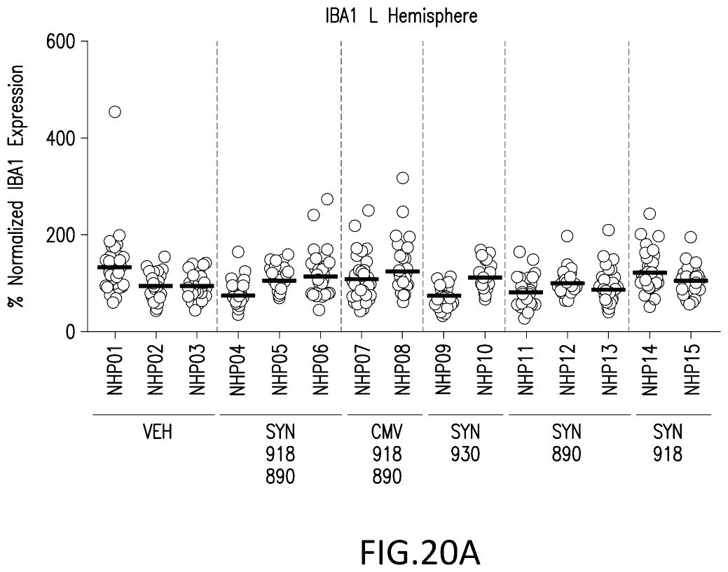

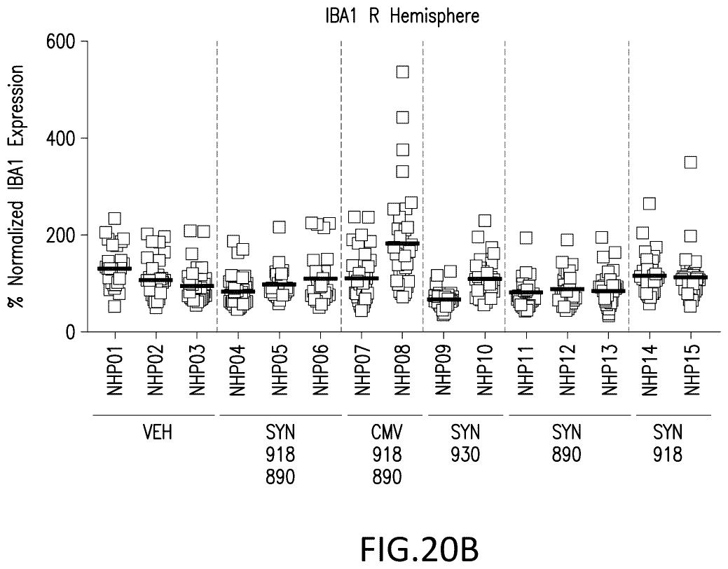

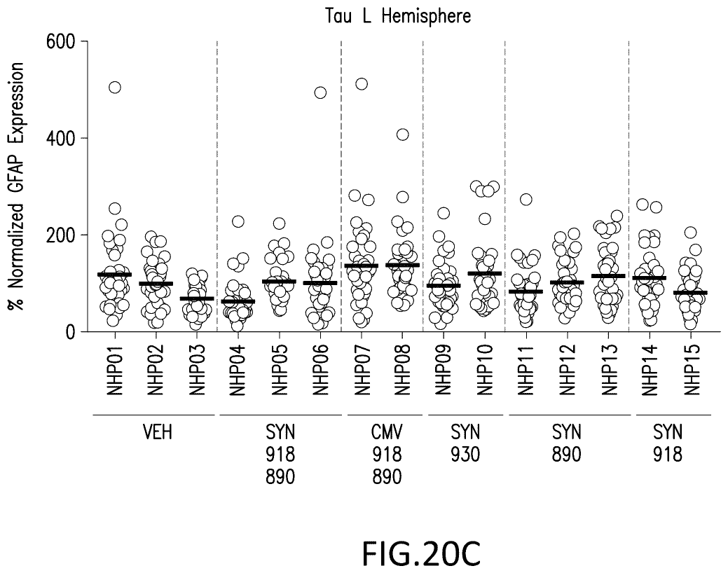

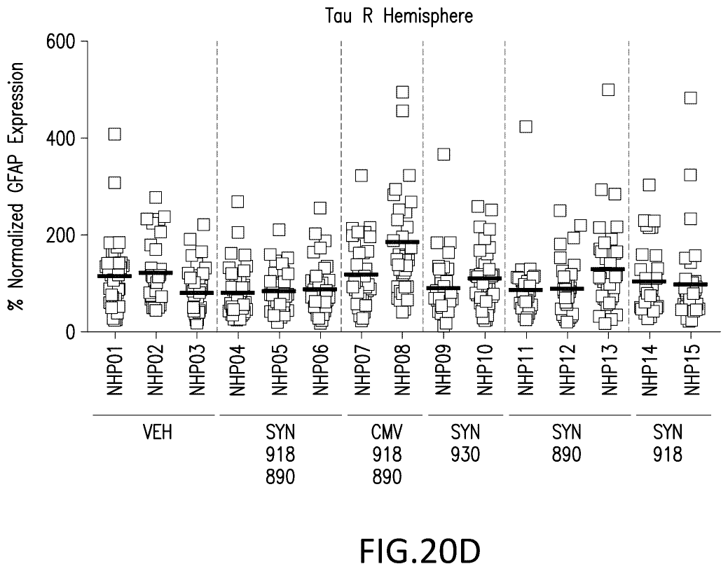

In addition, as shown in FIG. 20A through FIG. 20D, no major bulk effect for IBA1 (FIG. 19A through FIG. 19F) or GFAP (FIG. 19G through FIG. 19K) was observed for any treatment group.

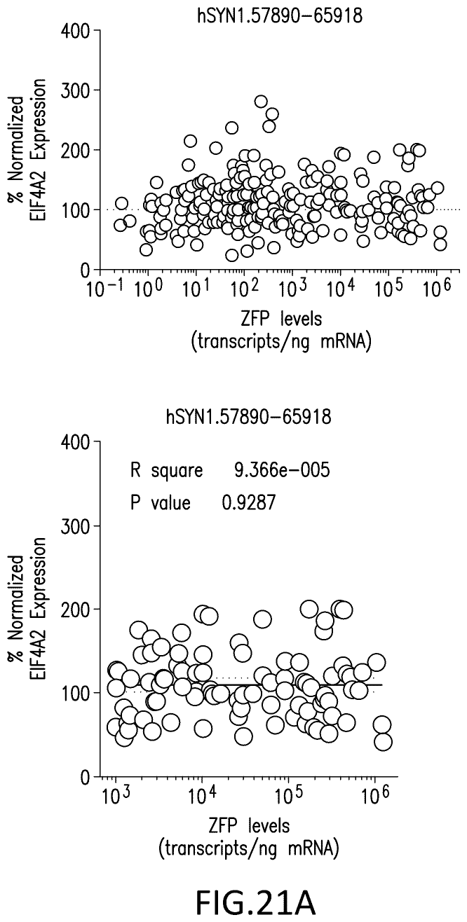

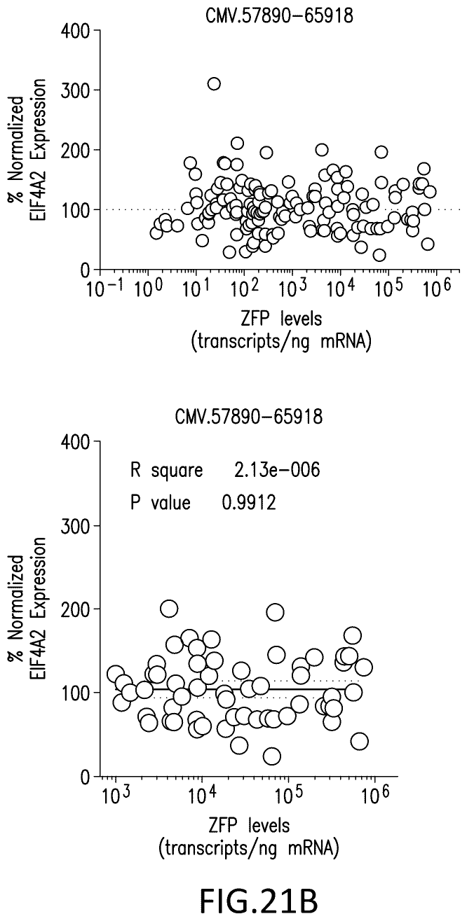

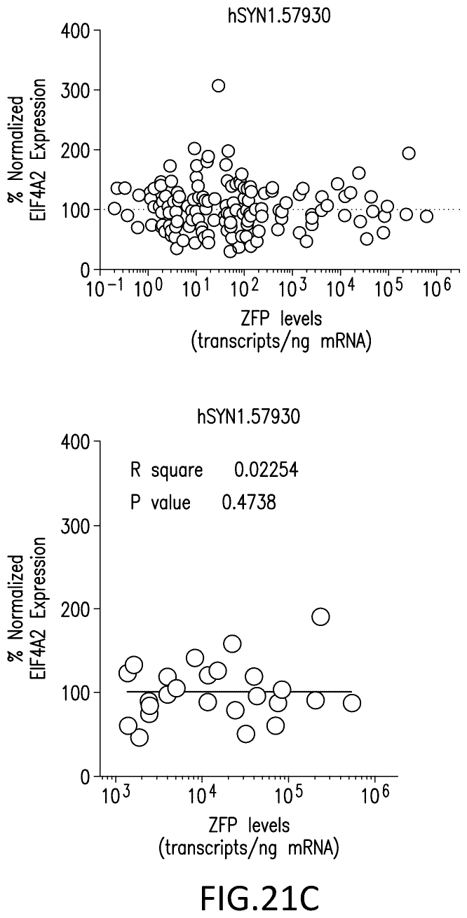

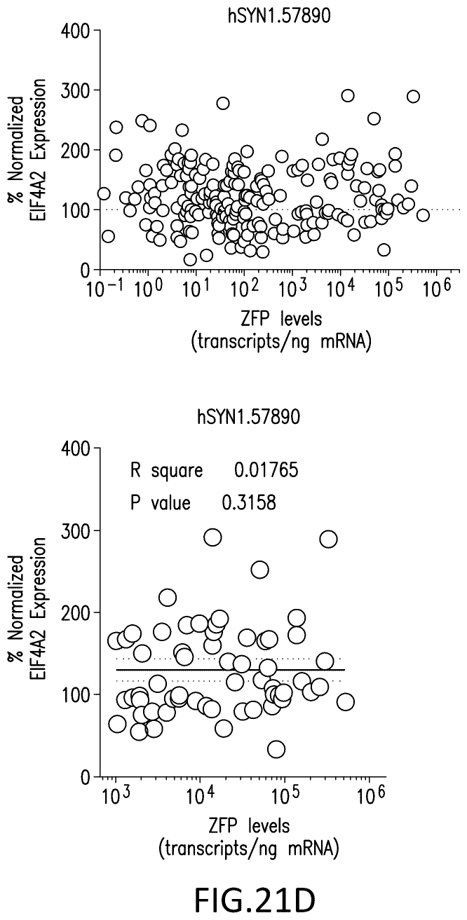

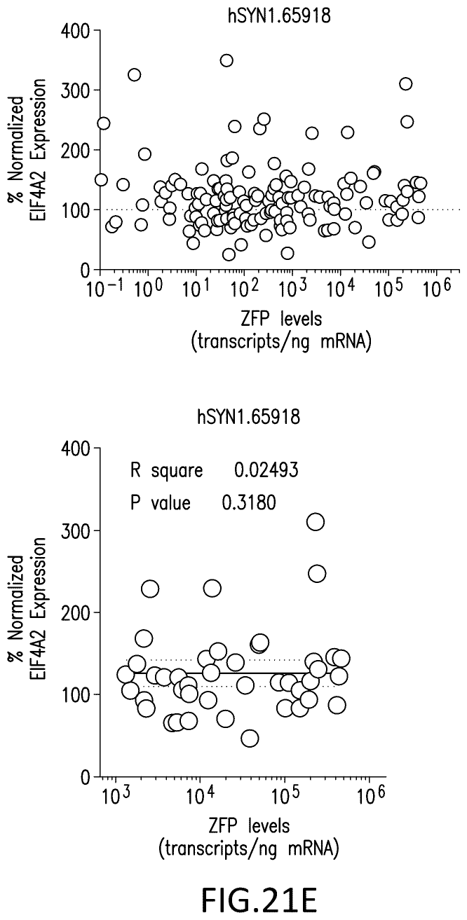

Furthermore, as shown in FIG. 21A through FIG. 21E, there was no correlation between the levels of a housekeeping gene (EIF4A2) and ZFP expression in primates treated with ZFP MAPT repressors. Any toxicity due to ZFP expression or tau reduction would be accompanied by a drop in E1F4A2 levels as ZFP levels increased.

Thus, synergistic tau repressors as described herein efficiently repress tau (up to 90% or more as compared to controls) in vivo without eliciting neuroinflammatory responses.

### Example 4: Tau Protein Levels

Tau protein levels are also tested cells and subjects receiving the repressors described herein.

Briefly, neurons (derived from induced pluripotent stem cells) were administered by AAV vectors as described above and tau protein levels in the cells or punches taken from the treated subjects were evaluated by ELISA using standard techniques. Briefly, neurons derived from human IPSCs were administered via AAV6 vectors encoding single ZFPs or the synergistic 65918/57890 combination at a dose of 1E5 VG per cells (n=4 replicates). Cells were cultured for 32 days and evaluated for tau protein levels by ELISA using standard techniques.

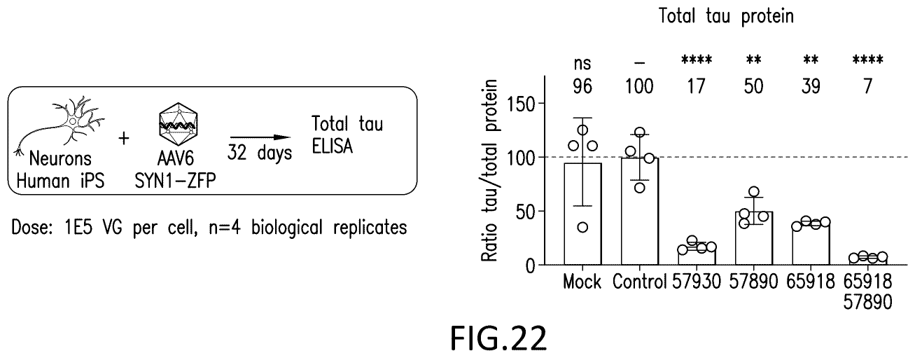

As shown in FIG. 22, the ratio of tau to total protein in human iPSC-derived neurons was significantly reduced in cells treated with tau repressors as described herein. In particular, the 57930 repressor alone reduced tau protein expression by more than 5-fold as compared to control; the 57890 repressor alone reduced tau protein expression by 2-fold; the 65918 repressor alone reduced tau expression by more than 2-fold; and the synergistic 65918-57890 reduced tau protein expression by more than 10-fold as compared to control. Tau protein levels were also evaluated in the CSF and/or brain homogenates of humanized tau mouse models treated as described in Example 2 above using standard ELISA (Thermo). A 50% reduction of the human tau protein was seen at 6 weeks with the 65918-57890 synergy pair as compared to the control (vehicle). The 65918-57890 pair also reduced human tau protein levels as compared to animals administered 57890 alone. Furthermore, tau protein levels are further reduced at later timepoints in animals, including 8-12 weeks or more after treatment.

Thus, tau repressors as described herein reduce tau protein levels in vitro and in vivo.

The studies demonstrate that the tau ZFP-TF reagents repress tau expression (including at therapeutic levels) in a primate brain.

All patents, patent applications and publications mentioned herein are hereby incorporated by reference for all purposes in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of synergistically modulating microtubule associated protein tau (MAPT) gene expression in a cell, the method comprising administering to a cell a composition comprising:
a first genetic modulator of the MAPT gene, the first genetic modulator comprising a first DNA-binding domain that binds to a first target site of at least 12 nucleotides in the MAPT gene and a first transcriptional regulatory domain or a first nuclease domain; and
a second genetic modulator of the MAPT gene, the second genetic modulator comprising a second DNA-binding domain that binds to a second target site of at least 12 nucleotides in the MAPT gene and a second transcriptional regulatory domain or a second nuclease domain wherein binding of the first genetic modulator to the first target site and binding of the second genetic modulator to the second target site results in synergistic-repression of MAPT gene expression.

2. The method of claim 1, wherein the composition is administered to a subject in need thereof.

3. The method of claim 2, wherein the administration is intracerebroventricular, intrathecal, intracranial, intravenous, intranasal, retro-orbital, or intracisternal.

4. The method of claim 2, wherein expression of the first and second genetic modulators of the composition reduce MAPT gene expression and/or tau protein levels in the brain of the subject by 50% or more as compared to untreated subjects.

5. The method of claim 1, wherein the first genetic modulator binds to capitalized nucleotides in tgGTGCTGGAGCTGGTGGGTggcggaga (SEQ ID NO: 1) and the second genetic modulator binds to capitalized nucleotides in cgGCAGAAGGTGGGcGGTGGCggcggcg (SEQ ID NO: 2),

6. The method of claim 1, wherein the first and the second DNA-binding domains comprise a zinc finger protein (ZFP), a TAL-effector domain protein (TALE) or single guide RNA.

7. The method of claim 1, wherein the first DNA-binding domain of the first genetic modulator is a first ZFP, the first ZFP comprises six zinc finger regions F1 to F6 comprising (i) SEQ ID NOs: 3, 4, 5, 6, 5, and 7, respectively.

8. The method of claim 1, wherein the second DNA-binding domain of the second genetic modulator is a second ZFP, the second ZFP comprises six zinc finger regions F1 to F6 comprising (i) SEQ ID Nos: 8-13, respectively, and (ii) an R-to-Q substitution at the −5 position of F1 and F5.

9. The method of claim 1, wherein the second DNA-binding domain of the second genetic modulator is a second ZFP, the second ZFP comprises six zinc finger regions F1 to F6 comprising SEQ ID Nos: 8-13, respectively.

10. The method of claim 1, wherein the first and the second genetic modulators are encoded by polynucleotides carried by one or more viral vectors.

11. The method of claim 10, wherein the one or more viral vectors are AAV vectors.

12. The method of claim 11, wherein one AAV vector comprises the polynucleotides encoding the first and the second ZFP.

13. The method of claim 12, wherein the AAV vector is an AAV9 vector.

14. The method of claim 10, wherein the one or more viral vectors comprise a CMV or synapsin (SYN) promoter.

15. The method of claim 1, wherein the first and the second genetic modulators are encoded by a single expression cassette in an AAV vector.

16. The method of claim 1, wherein the coding sequences for the first and the second genetic modulators are connected in frame by a coding sequence for a T2A peptide.

17. The method of claim 1, wherein the first and second genetic modulators cleave the MAPT gene.

18. The method of claim 17, wherein the composition further comprises a donor molecule that is integrated into the cleaved MAPT gene

19. A method of synergistically modulating MAPT expression in a cell, the method comprising administering to a cell a composition comprising a first and a second ZFP, wherein the first ZFP binds to capitalized nucleotides in tgGTGCTGGAGCTGGTGGGTggcggaga (SEQ ID NO: 1) and the second ZFP binds to capitalized nucleotides in cgGCAGAAGGTGGGcGGTGGCggcggcg (SEQ ID NO: 2) and binding of the first ZFP to SEQ ID NO: 1 and the second ZFP to SEQ ID NO: 2 results in synergistic-repression of MAPT gene expression.

20. A method of synergistically modulating MAPT expression in a cell, the method comprising administering to a cell a composition comprising a first and a ZFP, wherein the first ZFP binds to capitalized nucleotides in tgGTGCTGGAGCTGGTGGGTggcggaga (SEQ ID NO: 1) and the second ZFP binds to capitalized nucleotides in cgGCAGAAGGTGGGcGGTGGCggcggcg (SEQ ID NO: 2) and binding of the first ZFP to SEQ ID NO: 1 and the second ZFP to SEQ ID NO: 2 results in synergistic-repression of MAPT gene expression and wherein
(A) a first DNA-binding domain of the first ZFP comprises six zinc finger regions F1 to F6 comprising (i) SEQ ID NOs: 3, 4, 5, 6, 5, and 7, respectively; and
(B) a second DNA-binding domain of the second ZFP comprises six zinc finger regions F1 to F6 comprising SEQ ID Nos: 8-13, respectively.
